(19) United States
(12) Patent Application Publication
(10) Pub. No.: US 2023/0115675 A1
(43) Pub. Date: Apr. 13, 2023

(54) METHODS OF TREATING CANCER

(71) Applicants: The Broad Institute, Inc., Cambridge, MA (US); Dana-Farber Cancer Institute, Inc., Boston, MA (US)

(72) Inventors: Todd R. Golub, Newton, MA (US); Peter Tsvetkov, Cambridge, MA (US)

(21) Appl. No.: 17/905,013

(22) PCT Filed: Feb. 26, 2021
(86) PCT No.: PCT/US2021/019871

§ 371 Date: Aug. 25, 2022

Related U.S. Application Data
(60) Provisional application No. 62/983,300, filed on Feb. 28, 2020.

(51) Int. Cl. A61K 31/325; G01N 33/50; A61P 35/00

(52) U.S. Cl. CPC A61K 31/325; G01N 33/5079; A61P 35/00

(57) ABSTRACT

Provided herein are methods and compositions related to the treatment of cancer using copper ionophores.

### CROSS-REFERENCE TO A RELATED APPLICATION

This application claims the benefit of U.S. Provisional Application No. 62/983,300, filed on Feb. 28, 2020.

### BACKGROUND

Cancer cells demonstrate a remarkable ability to adapt to cytotoxic stressors and alter cell death pathways in order to survive. The initial ability of cancer cells to withstand cancer targeting therapy is associated with a shift in metabolism from glycolysis to increased mitochondrial metabolism. The shift to increased mitochondrial metabolism is associated with drug resistance in several cancer models. Moreover, this drug resistant state shows increased vulnerability to a copper ionophore named elesclomol. Elesclomol binds copper and promotes cell death, and cell death induced by elesclomol is dependent upon both intracellular and extracellular copper availability. The induction of cell death by elesclomol is highly augmented when cells shift from glycolysis to increased mitochondrial metabolism. Recently, multiple whole genome and metabolic gene-focused CRISPR/Cas9-based gene deletion screens have revealed that deletion of genes of the lipoic acid pathway and deletion of the gene encoding mitochondrial protein Ferredoxin 1 (FDX1) rescue cells from elesclomol induced cell death. Genetic and biochemical analysis further revealed that FDX1 is a crucial upstream regulator of the lipoic acid pathway and a key regulator of cell-death induction by copper ionophores such as elesclomol. These findings illuminate the role of copper and the lipoic acid pathway in promoting a shift to increased mitochondrial metabolism. This understanding of mitochondrial metabolism is of great importance in the treatment of cancer, especially for cancers in which there is an unmet need to combat pre-existing, intrinsic drug resistance and acquired drug resistance following drug exposure.

### SUMMARY

In certain aspects, provided herein are methods related to inhibiting growth or proliferation of a tumor and/or immune cell. In some embodiments, the methods comprise determining whether the tumor and/or immune cell is characterized by a level of protein lipoylation above a threshold level. In some embodiments, the methods comprise contacting the tumor and/or immune cell with a copper ionophore if the level of protein lipoylation is above the threshold level.

In certain aspects, provided herein are methods related to treating a subject for cancer that is refractory to treatment with an anti-cancer agent. In some embodiments, the methods comprise determining whether the cancer comprises a level of protein lipoylation above a threshold level. In some embodiments, the methods comprise conjointly administering a copper ionophore and the anti-cancer agent to the subject if the cancer is characterized by a level of protein lipoylation above the threshold level.

In certain aspects, provided herein are methods related to identifying a candidate anti-cancer agent. In some embodiments, the methods comprise a step of contacting a cell sample with a test agent. In some embodiments, the methods comprise a step of measuring a level of cellular protein lipoylation of the cell sample. In some embodiments, the methods comprise a step of identifying the test agent as a candidate anti-cancer agent if the level of cellular protein lipoylation is decreased as compared to a level of cellular protein lipoylation of a cell sample not contacted with the test agent.

In certain aspects, provided herein are methods related to determining increased mitochondrial metabolism in a tumor and/or immune cell. In some embodiments, the methods comprise staining for lipoic acid in the tumor and/or immune cell.

In certain aspects provided herein are methods related to identifying a candidate anti-cancer agent. In some embodiments, the methods comprise a step of incubating a cell sample with copper-supplemented media. In some embodiments, the methods comprise a step of contacting a cell sample with a test agent. In some embodiments, the methods comprise a step of measuring cell viability of the cell sample. In some embodiments, the methods comprise a step of identifying the test agent as a candidate anti-cancer agent if the level of cell viability is decreased as compared to a level of cell viability of a cell sample incubated with copper-supplemented media and not contacted with the test agent.

In certain aspects provided herein are methods related to identifying a candidate anti-cancer agent. Such methods may comprise a step of incubating a cell sample with a copper chelator, a step of contacting a cell sample with a test agent, and/or a step of measuring cell death of the cell sample. In such methods, the test agent may be identified as a candidate anti-cancer agent if the level of cell death is decreased as compared to a level of cell death of a cell sample incubated with a copper chelator and not contacted with the test agent.

In certain aspects, provided herein is a kit for identifying a candidate anti-cancer agent comprising a test agent, and an assay for measuring cellular protein lipoylation.

In certain aspects, provided herein is a kit for identifying a candidate anti-cancer agent comprising copper-supplemented media, a test agent, and an assay for measuring cell viability.

In certain aspects, provided herein is a kit for identifying a candidate anti-cancer agent comprising a copper chelator, a test agent, and an assay for measuring cell death.

### BRIEF DESCRIPTION OF THE DRAWINGS

FIG. 1 shows exemplary results demonstrating the role of copper in elesclomol killing.

FIG. 2 shows exemplary results of whole genome CRISPR rescue screens from Tsvetkov et al., Nat Chem Bio, 2019.

FIG. 3 shows PRISM biomarker analysis of elesclomol sensitivity from Tsvetkov et al., Nat Chem Bio, 2019.

FIG. 4 shows a ribbon structure of FDX1 with the elesclomol binding residues colored from Tsvetkov et al., Nat Chem Bio, 2019.

FIG. 5 shows the change in Fe—S assembly over time in the absence of elesclomol (control) or in the presence of 5×elesclomol or 10×elesclomol from Tsvetkov et al., Nat Chem Bio, 2019.

FIG. 6 shows exemplary results illustrating that elesclomol-Cu(II) is a neo-substrate of FDX1 from Tsvetkov et al., Nat Chem Bio, 2019.

FIG. 7 shows exemplary results indicating that elevated levels of mitochondrial metabolism predicts elesclomol sensitivity.

FIG. 8 shows exemplary results indicating that FDX1 regulates the lipoic acid pathway in cells, lipoic acid binds copper, and elesclomol reduces cellular lipoic acid.

FIG. 9 shows the promotion of copper dependent cell death in cancer cells by exemplary compounds.

FIG. 10 shows lipoic acid staining for elevated levels of mitochondrial metabolism in tumors that are sensitive to elesclomol.

FIG. 11 shows exemplary results indicating that mitochondrial copper toxicity leads to non-apoptotic cell death.

FIG. 12 shows exemplary results of PRISM Repurposing Secondary screen. The heat map is colored using the maximum Pearson correlation between all doses of each compound pair.

FIG. 13 shows viability of MON cells following treatment with increasing doses of indicated drugs in the presence of 10 μM FeCl₂, FeCl₃, ZnCl₂, NiCl, CuCl₂, or CoCl₂.

FIG. 14 shows viability of NCIH2030 cells following treatment with increasing doses of indicated drugs in the presence of 10 μM FeCl₂, FeCl₃, ZnCl₂, NiCl, CuCl₂, or CoCl₂.

FIG. 15 shows copper abundance in serum dictates elesclomol toxicity in BCPAP cells and PSN1 cells.

FIG. 16 shows copper abundance in serum dictates elesclomol toxicity in A549 cells.

FIG. 17 shows experimental setup of CRISPR-Cas9 positive selection screen in A549 cells using library targeting 3000 metabolism related genes (˜10 gRNAs per gene).

FIG. 18 shows deletion of FDX1 in A549 cells confers relative resistance to Elesclomol-Cu(II) and disulfiram-Cu(II) and deletion of LIAS or FDX1 in OVISE cells confer resistance to Elesclomol-Cu(II).

FIG. 19 shows FDX1 deletion correlates with the deletion of components of two distinct pathways.

FIG. 20 shows exemplary Western blot demonstrating deletion of FDX1 eliminates cellular lipoylated proteins in both OVISE cells and K562 cells.

FIG. 21 shows exemplary microscopy images demonstrating deletion of FDX1 eliminates cellular lipoylated proteins in both OVISE cells and K562 cells.

FIG. 22 shows proposed model of FDX1 function in lipoic acid pathway.

FIG. 23 shows the distribution of viability of 724 cell lines was examined by FIG. 24 shows experimental validation of FDX1 expression levels.

FIG. 25 shows Western Blot analysis of lipoylated proteins in resistant and sensitive cells.

FIG. 26 shows levels of lipoylation decrease following treatment A549 cells with 1 μM elesclomol (+CuCl₂).

FIG. 27 shows exemplary micrographs of control treatment or treatment with 100 nM of elesclomol for 24 hours of cells incubated with either 1 μM CuCl₂ or 1 μM CuCl₂.

FIG. 28 shows the viability of five ovarian cancer cell-lines following treatment with either elesclomol or elesclomol-Cu (1:1 ratio).

FIG. 29 shows a diagram of the apoptosis pathway; experimental inhibited targets are marked in red.

FIG. 30 shows exemplary viability results of 143B and 143B Rho0 cells grown in media containing either glucose or galactose after 72 hours.

FIG. 31 shows exemplary viability results of 143B and 143B Rho0 cells treated with indicated concentrations of elesclomol-Cu (1:1 ratio) after 72 hours.

FIG. 32A-F show exemplary viability results of HCM18 control cells or Bax and Bax deletion cells after treatment with indicate concentrations of different compounds (Pyrithione-Cu is Pyrithione-CuCl₂ (1:1); TMT-Cu is TMT-CuCl₂ (1:1); 8HQ-Cu is 8-HQ-CuCl₂ (1:1); Disulfiram-Cu is Disulfiram-CuCl₂ (1:1); NSC319726-Cu is NSC319726-Cu Cl₂ 1(1:1); AntiA is Antimycin A).

FIG. 32G-L show exemplary viability results of NCHIH2030 cells grown in the presence of 10 mM glucose or 10 mM galactose in the media after 72 hours (Pyrithione-Cu is Pyrithione-CuCl₂ (1:1); TMT-Cu is TMT-CuCl₂ (1:1); 8HQ-Cu is 8-HQ-CuCl₂ (1:1); Disulfiram-Cu is Disulfiram-CuCl₂ (1:1); NSC319726-Cu is NSC319726-Cu Cl₂ 1(1:1); AntiA is Antimycin A).

FIG. 32M-U show exemplary viability results of 143B and 143B Rho0 cells after 72 hours of treatment with indicated compounds at the specified concentrations (Pyrithione-Cu is Pyrithione-CuCl₂ (1:1); TMT-Cu is TMT-CuCl₂ (1:1); 8HQ-Cu is 8-HQ-CuCl₂ (1:1); Disulfiram-Cu is Disulfiram-CuCl₂ (1:1); NSC319726-Cu is NSC319726-Cu Cl₂ 1(1:1); AntiA is Antimycin A).

FIG. 33A-D show exemplary results of CRISPR-Cas9 gene knockout screens.

FIG. 34 shows a schematic of the lipoic acid pathway. The Fe—S cluster enzyme LIAS regulates the lysine lipoylation of enzymes, including DLAT.

FIG. 35 shows the average Log 2 fold change in metabolites between FDX1 KO K562 cells and AAVS1 K562 control cells separated by functional notations. Metabolites marked in orange are relevant to the lipoic acid pathway.

FIG. 36 shows exemplary Western blot of MON cells treated for 8 hours with indicated concentrations of elesclomol and analyzed for lipoylated protein content.

FIG. 37 shows exemplary quantification of lipoylated DLAT and DLST levels after treatment of cells with 40 nM elesclomol for 6 hours.

FIG. 38 shows exemplary results of FDX1 gene copy alteration analysis.

FIG. 39 shows results of a first drug screen with 1,583 compounds (left panel), and results of second drug screen with 851 compounds (middle panel), and exemplary copper ionophores (right panel). The first drug screen was conducted with 1,583 compounds in 4-5 doses in a model of proteasome inhibitor resistant cells versus control. The second drug screen was conducted with 851 compounds in 5 doses in a model of high OXPHOS versus glycolytic cell metabolism. Copper ionophores is the only class of compounds that preferentially kills cells in both High OXPHOS and PI resistant states.

FIG. 40 shows a schematic of the classic cell death pathways (apoptosis, necroptosis, and ferroptosis) and the cupropotosis cell death pathway. Cuproptosis is a new form of regulated cell death with distinct downstream regulators not shared with other regulated cell death programs such as apoptosis, ferroptosis and necroptosis.

FIG. 41 shows exemplary results of whole genome CRISPR/Cas9 deletion screens with two copper ionophores (left panel) and Venn diagram showing all the gene deletions that rescue from cuproptosis are related to FDX1 regulated protein lipoylation (right panel). Whole genome targeting CRISPR/Cas9 deletion screens with positive selection of two distinct copper bound ionophores (Elesclomol-Cu and Cu-DDC) revealed one common class of genes that when deleted promotes resistance to both compounds. These genes included FDX1, protein lipoylation enzymes (LIAS, LIPT1, and DLD) and subunits of the lipoylated protein complex pyruvate dehydrogenase (DLAT, PDHA1, and PDHB).

FIG. 42 shows a schematic of protein lipoylation pathway.

FIG. 43 shows a schematic of protein lipoylation pathway establishing that FDX1 is an upstream regulator of protein lipoylation (left panel) and DepMap analysis of gene deletion dependencies across hundreds of cancer cell lines (right panel). Analysis of the gene dependencies across hundreds of cancer cell lines revealed that FDX1 gene dependency is highly correlated with dependencies of proteins involved in lipoylation.

FIG. 44 shows exemplary graphs demonstrating protein levels of FDX1 and lipoylated proteins in elesclomol sensitive and resistant cell lines.

FIG. 45 shows an exemplary graph based on Immunohistochemistry (IHC) staining assay of lipoylated proteins across hundreds of tumors from distinct origin, establishing protein lipoylation as a protein biomarker for patient stratification.

FIG. 46 shows an exemplary IHC micrograph of gastrointestinal stromal tumor (GIST) (left panel) and circle chart of SDH (succinate dehydrogenase) deficient-GIST. Almost all SDHB deficient-GIST tumors exhibit high levels of protein lipoylation. Staining result revealed that specific GIST tumors that have depletion of the mitochondrial complex II protein SDHB (largely due to mutations in SDHA and SDHB genes) show particularly high levels of LA staining.

FIG. 47 shows an exemplary graph that establishes a biomarker positive mouse xenograft model.

FIG. 48 shows exemplary new copper ionophores based on elesclomol scaffold. Design is based on tractable structure-activity-relationship and structure-properties-relationship. 6 analogs of Elesclomol were synthesized with early tractable SAR.

FIG. 49 shows cytotoxicity of elesclomol-Cu(II) analogs in cells is dependent on their redox potential. The redox potential of the different elesclomol analogs when bound to copper is between −50 mV and −400 mV for the compound to have efficient cell killing (IC50<300 nM in MD-MBA455).

FIG. 50 shows results of PRISM Repurposing Secondary screen that includes growth-inhibition estimates for 1,448 drugs against 489 cell lines. Copper ionophores cluster in the drug space.

FIG. 51 shows schematic of drug discovery pipeline.

FIGS. 52A-C show exemplary graphs of tissue microarray (TMA) analysis of human breast carcinoma (n=67), ovarian carcinoma (n=84) and human non-small cell lung carcinoma (NSCLC) resections (N=57, 2 replicates per case) was stained with LA and FDX IHC and expression was scored semi-quantitatively by two pathologists (S.C., S.S.), showing a strong direct correlation between LA and FDX expression (mean±S.D.; p<0.0001).

FIGS. 52D-F show exemplary IHC staining micrographs. Representative cases of breast carcinoma (D), ovarian carcinoma (E) and NSCLC (F) with correlated low (top-row) and high (bottom-row) expression of LA and FDX1 by IHC (scale bars 20 μm).

FIGS. 53A-D show exemplary Western blots demonstrating deletion of FDX1 eliminates cellular lipoylated proteins (DLAT and DLST), in PSN1 (A-B), BCPAP (C) and ABC1 cells (D).

FIGS. 53E-H show exemplary graphs demonstrating deletion of FDX1 in both ABC1 (E-F) and PSN1 cells (G-H) abolishes respiration. The rate limiting lipoylation enzyme LIAS was used as a reference control.

FIG. 54A-E show exemplary plots of the log fold change versus the calculated p-value of FDX1 (A), DLAT (B), DLD (C) and LIAS (D) genes from the two whole genome CRISPR/Cas9 deletion screens in A549 cell lines for two concentrations of elesclomol-Cu (40 nM and 100 nM). (E) The correlation scores of FDX1 mRNA expression with the viability of 724 cancer cell lines as previously determined (4) for the different concentrations of elesclomol.

FIG. 55A-C shows an exemplary Western blot and graphs of single cell clones of ABC1 cells with either control AAVS1 or FDX1 gene deletions. Panel A shows FDX1, lipoylated DLAT and DLST and vinculin (as loading control). The relative sensitivity of each single cell clone to elesclomol (in the presence of 1 uM CuCl₂) was measured from bottom of Panel A in Panel B. Panel C shows the correlation of elesclomol EC50 and relative FDX1 protein levels. Decrease of FDX1 beyond certain threshold increases the cell resistance to elesclomol.

FIG. 56A-F shows exemplary graphs examining the efficacy of elesclomol in conditions that mimic the pharmokinetic (PK) properties previously described in mouse models. Panel A demonstrates biomarker positive (high FDX1 gene expression) cells are more sensitive to elesclomol than biomarker negative cells. Panels B and C show the viability of biomarker positive cells—ABC1 (Panel B) and biomarker negative—A549 (Panel C) cells measured at the indicated time points after a 2 hour pulse of 100 nM elesclomol in the presence of 1 μM CuCl₂ in the media. Panels D and Panel E show the relative changes in metabolites as measured in ABC1(D) and A549 (E) 24 hours after a 2 hour pulse treatment with 100 nM elesclomol. Panel F shows the changes in Sedoheptulose-7-phosphate in ABC1 cells following a pulse treatment with 100 nM elesclomol. Panel G shows the changes in Sedoheptulose-7-phosphate in control AAVS1 and FDX1 KO ABC1 cells treated for 24 hours with 1 nM elesclomol.

### DETAILED DESCRIPTION

### General

In certain aspects, the methods and compositions provided herein are based, in part, on the discovery that tumor cells expressing certain biomarkers can be effectively treated with a copper ionophore. Exemplary copper ionophores include elesclomol and disulfiram, which were previously disclosed in US Patent Application 2018/0353445. Provided herein are methods of measuring levels of certain biomarkers, such as lipoylated proteins (e.g., lipoyl-DLAT, lipoyl-DLAT, lipoyl-DLST, lipoyl-GCSH, lipoyl-DBT) and lipoic acid biosynthesis proteins (e.g., LIAS, LIPT1, LIPT2, DLD) in tumor cells. Also provided herein are methods of measuring biomarkers in conjunction with certain mitochondrial proteins that bind copper ionophores (e.g., FDX1, ALDHA1, ALDH2). In certain aspects, the methods and compositions provided herein may be advantageously used to inhibit growth or proliferation of a tumor, treat refractory cancer, and/or identify a candidate anti-cancer agent. For example, in certain embodiments the methods and compositions provided herein are especially useful for treatment of cancers resistant to targeted drug therapy.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

The term “agent” refers to any substance, compound (e.g., molecule), supramolecular complex, material, or combination or mixture thereof.

The term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as VH) and a heavy chain constant region. Each light chain includes a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The term “biological sample,” “tissue sample,” or simply “sample” each refers to a collection of cells obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject.

The term “binding” or “interacting” refers to an association, which may be a stable association, between two molecules, due to, for example, electrostatic, hydrophobic, ionic and/or hydrogen-bond interactions under physiological conditions.

The term “measuring” refers to determining the presence, absence, quantity amount, or effective amount of a substance in a sample, including the concentration levels of such substances.

The term “refractory” refers to cancer that does not respond to the treatment. The lack of response can be assessed by, for example, lack of inhibition of tumor growth or increased tumor growth; lack of reduction in the number of tumor cells or an increase in the number of tumor cells; increased tumor cell infiltration into adjacent peripheral organs and/or tissues; increased metastasis; decrease in the length of survival following treatment; and/or mortality. The cancer may be resistant at the beginning of treatment or it may become resistant during treatment.

As used herein, the term “subject” means a human or non-human animal selected for treatment or therapy.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

In certain embodiments, therapeutic compounds may be used alone or conjointly administered with another type of therapeutic agent (e.g., an immuno-oncology agent or a chemotherapeutic agent disclosed herein). As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of therapeutic compounds with one or more additional therapeutic agent(s) (e.g., one or more additional chemotherapeutic agent(s)) provides improved efficacy relative to each individual administration of the compound (e.g., copper ionophore) or the one or more additional therapeutic agent(s). In certain such embodiments, the conjoint administration provides an additive effect, wherein an additive effect refers to the sum of each of the effects of individual administration of the therapeutic compound and the one or more additional therapeutic agent(s).

### Pharmaceutical Compositions and Administration

In certain embodiments, provided herein are pharmaceutical compositions and methods of using pharmaceutical compositions. In some embodiments, the pharmaceutical compositions provided herein comprise a copper ionophore (e.g., elesclomol, disulfiram). In some embodiments, the pharmaceutical compositions provided herein comprise an anti-cancer agent (e.g., a chemotherapeutic agent, an immune checkpoint inhibitor, an EGFR inhibitor, or a proteasome inhibitor).

This invention also provides compositions and methods that may be utilized to treat a subject in need thereof. In certain embodiments, the subject is a mammal such as a human, or a non-human mammal. In some embodiments, the subject has cancer, optionally a drug resistant cancer, e.g., a drug resistant cancer with biomarkers of lipoylation. When administered to a subject, such as a human, the composition or the compound is preferably administered as a pharmaceutical composition comprising, for example, a therapeutic compound and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In certain embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

In certain embodiments, the pharmaceutical compositions provided herein comprise a pharmaceutically acceptable carrier. The phrase “pharmaceutically acceptable carrier” as used herein means a pharmaceutically acceptable material, composition or vehicle, such as a liquid or solid filler, diluent, excipient, solvent or encapsulating material. A pharmaceutically acceptable carrier can contain physiologically acceptable agents that act, for example, to stabilize, increase solubility or to increase the absorption of a compound. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a therapeutic compound. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In certain embodiments, the pharmaceutical compositions provided herein can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound with the carrier and, optionally, one or more accessory ingredients. In general, the formulations are prepared by uniformly and intimately bringing into association a compound with liquid carriers, or finely divided solid carriers, or both, and then, if necessary, shaping the product.

Formulations suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

In certain embodiments, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular subject, composition, and mode of administration, without being toxic to the subject.

If desired, the effective daily dose of the active compound may be administered as one, two, three, four, five, six or more sub-doses administered separately at appropriate intervals throughout the day, optionally, in unit dosage forms. In certain embodiments, the active compound may be administered two or three times daily. In some embodiments, the active compound will be administered once daily.

In certain embodiments, compounds may be used alone or conjointly administered with another type of therapeutic agent (e.g., an immuno-oncology agent or a chemotherapeutic agent disclosed herein). As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of therapeutic compounds with one or more additional therapeutic agent(s) (e.g., one or more additional chemotherapeutic agent(s)) provides improved efficacy relative to each individual administration of the compound (e.g., copper ionophore) or the one or more additional therapeutic agent(s). In certain such embodiments, the conjoint administration provides an additive effect, wherein an additive effect refers to the sum of each of the effects of individual administration of the therapeutic compound and the one or more additional therapeutic agent(s).

In certain embodiments, pharmaceutically acceptable salts of compounds can be used in the methods provided herein. Suitable salts include, but are not limited to, HCl, trifluoroacetic acid (TFA), maleate, alkyl, dialkyl, trialkyl or tetra-alkyl ammonium salts. In certain embodiments, contemplated salts include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts include, but are not limited to, Na, Ca, K, Mg, Zn, copper, cobalt, cadmium, manganese, or other metal salts.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In some embodiments, the therapeutic compound used in the methods herein is a copper ionophore. Exemplary copper ionophores are provided in Table 1.

TABLE 1
Exemplary Copper Ionophores

| Compound Name | Structure |
|---|---|
| Pyrithione Zinc | 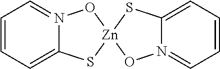 |
| Tetramethylthiuram-monosulfide | 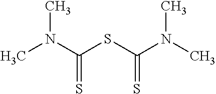 |
| Oxyquinoline (8HQ) | 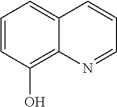 [SMILES: Oc1cccc2cccnc12] |
| Elesclomol | 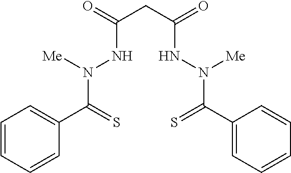 [SMILES: CN(NC(=O)CC(=O)NN(C)C(=S)c1ccccc1)C(=S)c1ccccc1] |
| Disulfiram | 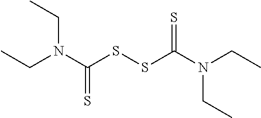 [SMILES: CCN(CC)C(=S)SSC(=S)N(CC)CC] |
| Thiram | 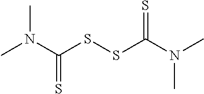 |
| Cu(GTSM) | 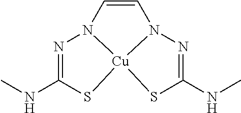 |
| NSC-319726 | 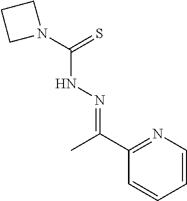 [SMILES: C/C(=N\NC(=S)N1CCC1)c1ccccn1] |
| FR-122047 | 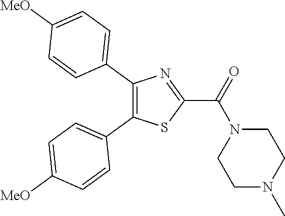 [SMILES: COc1ccc(-c2nc(C(=O)N3CCN(C)CC3)sc2-c2ccc(OC)cc2)cc1] |
| Cu(isapn) | 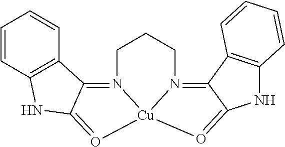 |

In some embodiments, the therapeutic compound is a Paullone-based complex, two representative structures of which are shown below.

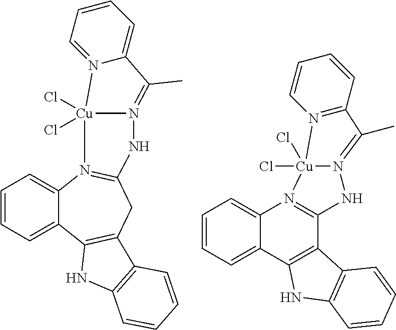

In some embodiments, the therapeutic compound is a Casiopeina-based complex, two representative structures of which are shown below.

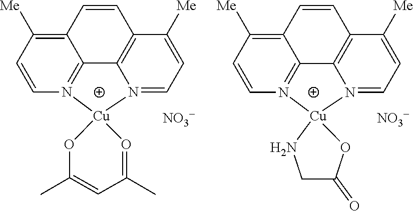

In some embodiments, the therapeutic compound is a Bis(thiosemicarbazone) Cu complex, a representative structure of which is shown below.

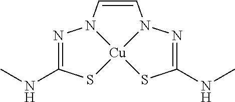

In some embodiments, the therapeutic compound is a Isatin-Schiff-based complex, two representative structures of which are shown below.

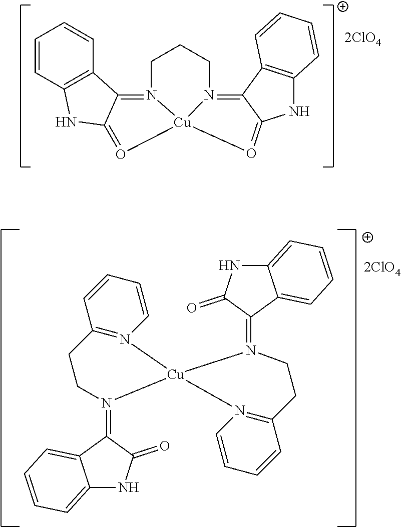

In some embodiments, the therapeutic compound is a (D-glucopyranose)-4-phenylthiosemicarbazide Cu complex, a representative structure of which is shown below.

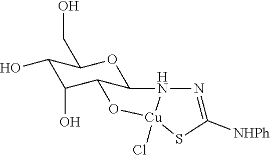

In some embodiments, the therapeutic compound is a BCANa₂, a representative structure of which is shown below.

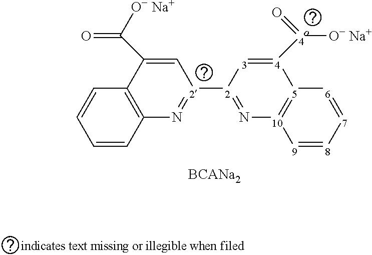

In some embodiments, the therapeutic compound is a BCSNa₂, a representative structure of which is shown below.

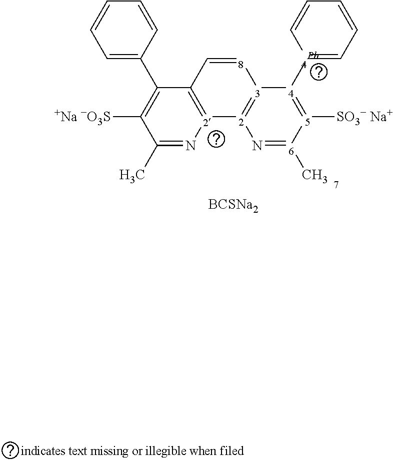

In some embodiments, the therapeutic compound is a BCSANa₂, the general structure of which is shown below.

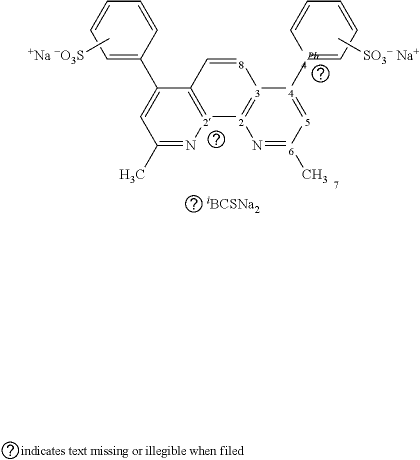

In some embodiments, the therapeutic compound is PTA, the structure of which is shown below.

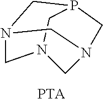

In some embodiments, the therapeutic compound is DAPTA, the structure of which is shown below.

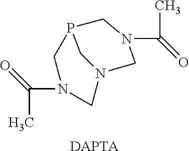

In some embodiments, the therapeutic compound is a soluble thiosemicarbazone complex, representative structures of which are shown below.

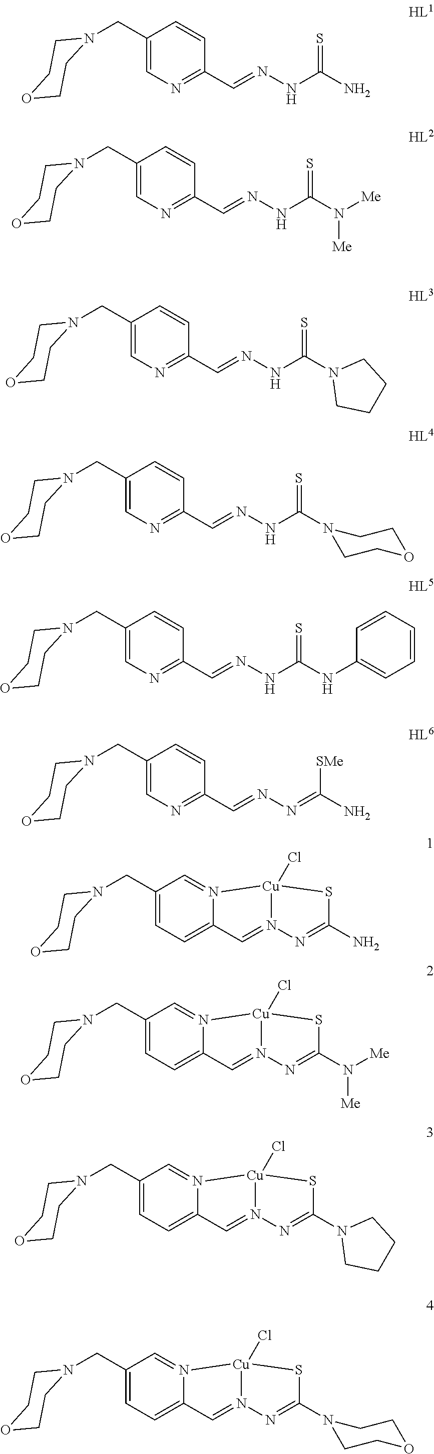

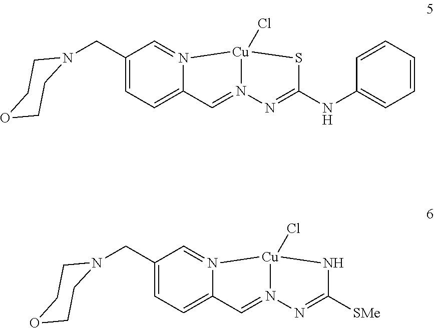

In some embodiments, the therapeutic compound is a Schiff base complex, representative structures of which are shown below.

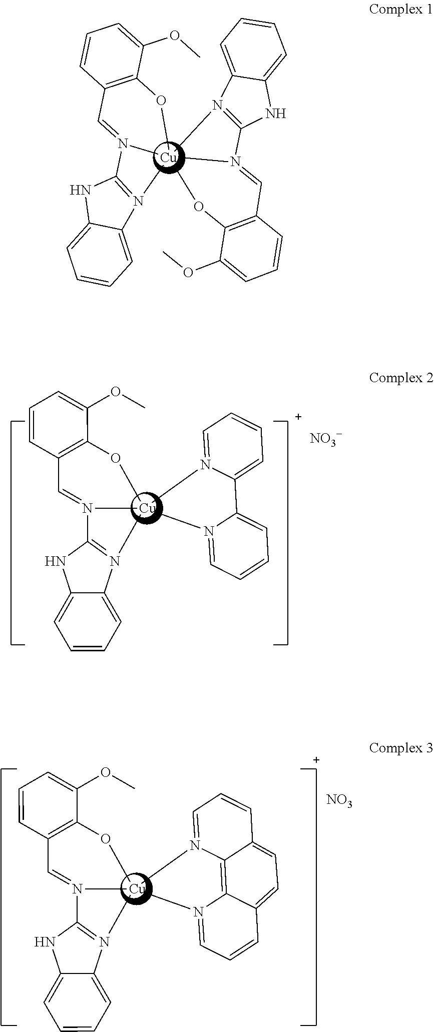

In some embodiments, the therapeutic compound is a dithiocarbamate. In some embodiments the dithiocarbamate is tetraethylthiuram disulfide (disulfiram; CAS Registry Number 97-77-8), the structure of which is shown below.

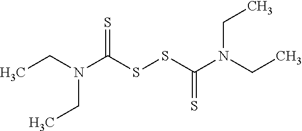 [SMILES: CCN(CC)C(=S)SSC(=S)N(CC)CC]

In some embodiments the therapeutic compound is a disulfiram analog referred to as compound 339 (Sharma, V., et al. Mol Carcinog. 2015 Nov. 24. doi: 10.1002/mc.22433. [Epub ahead of print]). In some embodiments the compound is a disulfiram metabolite. In some embodiments the dithiocarbamate is pyrrolidine dithiocarbamate (PDTC).

In some embodiments, the therapeutic compound is a bis(thio-hydrazide amide). Exemplary bis(thio-hydrazide amides) are described in U.S. Pat. Nos. 6,762,204, 6,800,660, 6,924,312, 7,001,923, 7,037,940, U.S. Patent Application Publication Nos. 20030045518, 20030119914, 20030195258, and 20080119440. For example, in some embodiments the bis(thio-hydrazamide amide) is represented by any of structural formulae (I)-(VI) disclosed in U.S. Pat. No. 6,800,660, with the various variables and chemical terms defined as described therein. In some embodiments the bis(thio-hydrazamide amide) is represented by any of structural formulae I, II, IIIa, IIIb, IVa, IVb, or V disclosed in U.S. patent: application Publication No. 20080119440 (US20080119440) with the various variables and chemical terms defined and as described therein. For convenience, definitions of certain such terms are set forth below. In some embodiments, for example, the compound has the following structural formula (formula 1 as described in US20080119440):

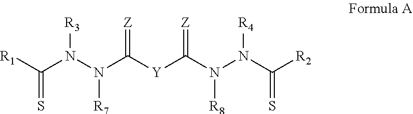

wherein Y is a covalent bond or an optionally substituted straight chained hydrocarbyl group, or, Y, taken together with both >C═Z groups to which it is bonded, is an optionally substituted aromatic group; R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic ring optionally fused to an aromatic ring; R₇ and R₅ are independently —H, an optionally substituted aliphatic group, or an optionally substituted aryl group; and Z is O or S. In certain embodiments Z is O. In certain embodiments R₁, R₂, or both, are optionally substituted phenyl groups. In some embodiments R₁ and R₂ are the same. In some embodiments R₃ and R₄ are lower alkyl groups, e.g., methyl groups. In some embodiments R₃ and R₄ are the same. In certain embodiments Y is CH₂. In certain embodiments Z is O; R₁, R₂, or both, are optionally substituted phenyl groups, which are optionally the same; R₃ and R₄ are lower alkyl groups (C1-C8 straight chained or branched alkyl group or a C3-C8 cyclic alkyl group), e.g., methyl groups, which are optionally the same; and Y is CH₂. In certain embodiments Z is O; R₁, R₂, or both, are optionally substituted cyclopropyl groups, which are optionally the same; R₃ and R₄ are lower alkyl groups (C1-C8 straight chained or branched alkyl group or a C3-C8 cyclic alkyl group), e.g., methyl groups, which are optionally the same; and Y is CH₂. In certain embodiments R₃, R₄, or both, are cyclopropyl. In certain embodiments R₃, R₄, or both, are methylcyclopropyl. As used herein, single bonds are represented by a dash symbol (-) and double bonds are represented by an equal sign (=).

In some embodiments the bis(thio-hydrazamide amide) has the following formula. (formula Mb as described in US20080119440):

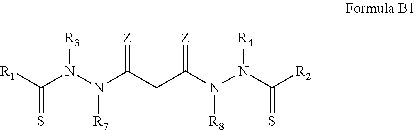

wherein Z, R₁, R₂, R₃, R₄, R₇, and R₅ are as defined above for Formula A. In some embodiments the bis(thio-hydrazamide amide) has the following formula (formula V as described in US20080119440):

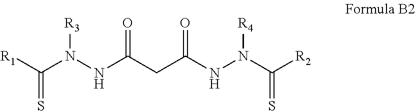

wherein R₁, R₂, R₃, and R₄ are as defined above for Formula A.

In some embodiments of the compounds of Formula B1 or B2, R₁ and R₂ are both phenyl, and R₃ and R₄ are both O—CH₃-phenyl; R₁ and R₂ are both O—CH₃C(O)O-phenyl, and R₃ and R₄ are phenyl; R₁ and R₂ are both phenyl, and R₃ and R₄ are both methyl; R₁ and R₂, are both phenyl, and R₃ and R₄ are both ethyl; R₁ and R₂ are both phenyl, and R₃ and R₄ are both n-propyl; R₁ and R₂ are both p-cyanophenyl, and R₃ and R₄ are both methyl; R₁ and R₂ are both p-nitro phenyl, and R₃ and R₄ are both methyl; R₁ and R₂ are both 2,5-dimethoxyphenyl, and R₃ and R₄ are both methyl; R₁ and R₂ are both phenyl, and R₃ and R₄ are both n-butyl; R₁ and R₂, are both p-chlorophenyl, and R₃ and R₄ are both methyl; R₁ and R₂ are both 3-nitrophenyl, and R₃ and R₄ are both methyl; R₁ and R₂ are both 3-cyanophenyl, and R₃ and R₄ are both methyl; R₁ and R₂ are both 3-fluorophenyl, and R₃ and R₄ are both methyl; R₁ and R₂ are both 2-furanyl, and R₃ and R₄ are both phenyl; R₁ and R₂ are both 2-methoxyphenyl, and R₃ and R₄ are both methyl; R₁ and R₂ are both 3-methoxyphenyl, and R₃ and R₄ are both methyl; R₁ and R₂ are both 2,3-dimethoxyphenyl, and R₃ and R₄ are both methyl; R₁ and R₂ are both 2-methoxy-5-chlorophenyl, and R₃ and R₄ are both ethyl; R₁ and R₂ are both 2,5-difluorophenyl, and R₃ and R₄ are both methyl; R₁ and R₂ are both 2,5-dichlorophenyl, and R₃ and R₄ are both methyl; R₁ and R₂ are both 2,5-dimethylphenyl, and R₃ and R₄ are both methyl; R₁ and R₂ are both 2-methoxy-5-chlorophenyl, and R₃ and R₄ are both methyl; R₁ and R₂ are both 3,6-dimethoxyphenyl, and R₃ and R₄ are both methyl; R₁ and R₂ are both phenyl, and R₃ and R₄ are both 2-ethylphenyl; R₁ and R, are both 2-methyl-5-pyridyl, and R₃ and R₄ are both methyl; or R₁ is phenyl; R₂ is 2,5-dimethoxyphenyl, and R₃ and R₄ are both methyl; R₁ and R₂ are both methyl, and R₃ and R₄ are both p-CF₃-phenyl; R₁ and R₂ are both methyl, and R₃ and R₄ are both O—CH₃-phenyl; R₁ and R₂ are both —(CH₂)₃COOH; and R₃ and R₄ are both phenyl; R₁ and R₂ are both represented by the following structural formula:

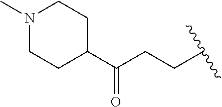 [SMILES: *CCC(=O)C1CCN(C)CC1]

and R₃ and R₄ are both phenyl; R₁ and R₂ are both n-butyl, and R₃ and R₄ are both phenyl; R₁ and R₂ are both n-pentyl, R₃ and R₄ are both phenyl; R₁ and R₂ are both methyl, and R₃ and R₄ are both 2-pyridyl; R₁ and R₂ are both cyclohexyl, and R₃ and R₄ are both phenyl; R₁ and R₄ are both methyl, and R₃ and R₄ are both 2-ethylphenyl, R₁ and R₂ are both methyl, and R₃ and R₄ are both 2,6-dichlorophenyl; R₁-R₄ are all methyl; R₁ and R₂ are both methyl, and R₃ and R₄ are both t-butyl; R₁ and R₂ are bath ethyl, and R₃ and R₄ are both methyl; R₁ and R₂ are both t-butyl, and R₃ and R₄ are both methyl; R₁ and R₂ are both cyclopropyl, and R₃ and R₄ are both methyl; R₁ and R₂ are both cyclopropyl, and R₃ and R₄ are both ethyl; R₁ and R₂ are both 1-methylcyclopropyl, and R₃ and R₄ are both methyl; R₁ and R₂ are both 2-methylcyclopropyl, and R₃ and R₄ are both methyl; R₁ and R₂ are both 1-phenylcyclopropyl, and R₃ and R₄ are both methyl; R₁ and R₂ are both 2-phenylcyclopropyl, and R₃ and R₄ are both methyl; R₁ and R₂ are both cyclobutyl, and R₃ and R₄ are both methyl; R₁ and R₂ are both cyclopentyl, and R₃ and R₄ are both methyl; R₁ is cyclopropyl, R₂ is phenyl, and R₃ and R₄ are both methyl. In some embodiments, for example, R₁ and R₂ are a substituted or unsubstituted phenyl group and R₃ and R₄ are a lower alkyl group (e.g., methyl), wherein in some embodiments (i) R₁ and R₂ are the same; (ii) R₃ and R₄ are the same; or (iii) R₁ and R₂ are the same and R₃ and R₄ are the same.

In some embodiments the bis(thio-hydrazarnide amide) has the following formula (formula Ma as described in US20080119440):

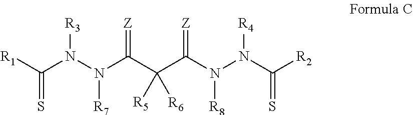

wherein Z, R₁, R₂, R₃, R₄, R₇, and R₅ are as defined above for Formula A, and wherein R₅ and R₆ are independently —H or lower alkyl, e.g., methyl, ethyl, propyl. In some embodiments Z is O. In some embodiments R₁, R₂, R₃, and R₄ are as defined for Formula B1 or B2 and R₅ and R₆ are independently —H or lower alkyl, e.g., methyl, ethyl, propyl.

As used herein, unless indicated otherwise, consistent with US20080119440, an “alkyl group” is a saturated straight or branched chain linear or cyclic hydrocarbon group. Typically, a straight chained or branched alkyl group has from 1 to about 20 carbon atoms, preferably from 1 to about 10, and a cyclic alkyl group has from 3 to about 10 carbon atoms, preferably from 3 to about 8. An alkyl group is preferably a straight chained or branched alkyl group, e.g., methyl, ethyl, n-propyl, iso-propyl, n-butyl, sec-butyl, tert-butyl, pentyl, hexyl, pentyl or octyl, or a cycloalkyl group with 3 to about 8 carbon atoms. A C1-C8 straight chained or branched alkyl group or a C3-C8 cyclic alkyl group is also referred to as a “lower alkyl” group, as noted above.

A “straight chained hydrocarbyl group” is an alkylene group, i.e., —(CH₂)_{y}—, with one or more (preferably one) internal methylene groups (—(CH₂)—) optionally replaced with a linkage group. y is a positive integer (e.g., between 1 and 10), preferably between 1 and 6 and more preferably 1 or 2. A “linkage group” in this context refers to a functional group which replaces a methylene in a straight chained hydrocarbyl. Examples of suitable linkage groups include a ketone (—C(O)—), alkene, alkyne, phenylene, ether (—O—), thioether (—S—), or amine (—N(R₃)—), wherein R, is defined below.

An “aliphatic group” is a straight chained, branched or cyclic non-aromatic hydrocarbon which is completely saturated or which contains one or more units of unsaturation. Typically, a straight chained or branched aliphatic group has from 1 to about 20 carbon atoms, preferably from 1 to about 10, and a cyclic aliphatic group has from 3 to about 10 carbon atoms, preferably from 3 to about 8. An aliphatic group is preferably a straight chained or branched alkyl group, e.g., methyl, ethyl, n-propyl, iso-propyl, n-butyl, sec-butyl, tert-butyl, pentyl, hexyl, pentyl or octyl, or a cycloalkyl group with 3 to about 8 carbon atoms.

The term “aromatic group” may be used interchangeably with “aryl”, “aryl ring,” “aromatic ring”, “aryl group” and “aromatic group”. Aromatic groups include carbocyclic aromatic groups such as phenyl, naphthyl, and anthracvl, and heteroaryl groups such as imidazolyl, thienyl, furanyl, pyridyl, pyrimidyl, pyrazolyl, pyrroyl, pyrazinyl, thiazole, oxazolyl, and tetrazole. The term “heteroaryl group” may be used interchangeably with “heteroaryl”, “heteroaryl ring”, “heteroaromatic ring” and “heteroaromatic group”. Heteroarvl groups are aromatic groups that comprise one or more heteroatom, such as sulfur, oxygen and nitrogen, in the ring structure. Preferably, heteroaryl aroups comprise from one to four heteroatoms. Aromatic groups also include fused polycyclic aromatic ring systems in which a carbocyclic aromatic ring or heteroaryl ring is fused to one or more other heteroaryl rings. Examples include benzothienyl, indolyl, quinolinyl, benzothiazole, benzooxazole, benzimidazole, quinolinyl, isoquinolinyl and isoindolyl. Non-aromatic heterocyclic rings are non-aromatic rings which include one or more heteroatoms such as nitrogen, oxygen or sulfur in the ring. The ring can be five, six, seven or eight-membered. Preferably, heterocyclic groups comprise from one to about four heteroatoms. Examples include tetrahydrofuranyl, tetrahydrothiophenyl, morpholino, thiomorpholino, pyrrolidinyl, piperazinyl, piperidinyl, and thiazolidinyl.

Examples of suitable substituents for an aryl group or an aliphatic group are described in US Patent Application Publication No. 20080119440. For example, in some embodiments a substituent is a group selected from R^{a}, —OH, —Br, —Cl, —F, —O—COR^{a}, —CN, —NCS, —NO₂, —COOH, —NH₂, —N(R^{a}R^{b}), —COOR^{a}, —CHO, —CONH₂, —CONHR^{a}, —CON(R^{a}R^{b}), —NHCOR^{a}, —NRCCOR^{a}, —NHCONH₂, —NHCONR^{a}H, —NHCON(R^{a}R^{b}), —NR^{c}CONH₂, —NRCCON^{a}H, —NR^{c}CON(R^{a}R^{b}), —C(═NH)—NH₂, —C(═NH)—NHW, —C(═NH)—N(R^{a}R^{b}), —C(NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NH^{a}, —NH—C(═NR^{c})—N(R^{a}R^{b}), —NR^{d}—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHNR^{a}R^{b}, —SO₂NH₂, —SO₂NHR^{a}, —SO₂NR^{a}R^{b}, —CH═CHR^{a}, —CH═CR^{a}R^{b}, —CR^{c}CR^{a}R^{b}, —CR^{c}═CHR^{a}, —CR^{c}═CR^{a}R^{b}, —CCR^{a}, —SH, —SR^{a}, —S(O)R^{a}, —S(O)₂R^{a}, wherein R^{a}-R^{d} are each independently an alkyl group, aromatic group, non-aromatic heterocyclic group; or, —N(R^{a}R^{b}), taken together, form an optionally substituted non-aromatic heterocyclic group, wherein the alkyl, aromatic and non-aromatic heterocyclic group represented by R^{a}-R^{d} and the non-aromatic heterocyclic group represented by —N(R^{a}R^{b}) are each optionally and independently substituted with one or more groups represented by R^{#}, wherein R is R⁺, —O(haloalkyl), —NO₂, —CN, —NCS, —N(R⁺)₂, —NHCO₂R⁺, —NHC(O)R⁺, —NHNHC(O)R⁺, —NHC(O)N(R⁺)₂, —NHNHC(O)N(R⁺)₂, —NHNHCO₂R⁺, —C(O)C(O)R⁺, —C(O)CH₂C(O)R⁺, —CO₂R⁺, —C(O)R⁺, C(O)N(R⁺)₂, —OC(O)R⁺, —OC(O)N(R⁺)₂, —S(O)₂R⁻, —SO₂N(R⁺)₂, —S(O)R⁺, —NHSO₂N(R⁺)₂, —NHSO₂R⁺, —C(═S)N(R⁺)₂, or —C(═NH)—N(R⁺)₂; wherein R⁺ is —H, a C1-C4 alkyl group, a monocyclic heteroaryl group, a non-aromatic heterocyclic group or a phenyl group optionally substituted with alkyl, haloalkyl, alkoxy, haloalkoxy, halo, —CN, —NO₂, amine, alkylamine or dialkylamine; or —N(R⁺)₂ is a non-aromatic heterocyclic group, provided that non-aromatic heterocyclic groups represented by R⁺ and —N(R⁺)₂ that comprise a secondary ring amine are optionally acylated or alkylated.

In certain embodiments substituents for a phenyl group, such as phenyl groups that may be present at positions represented by R₁-R₄, include C1-C4 alkyl, C1-C4 alkoxy, C1-C4 haloalkyl, C1-C4 haloalkoxy, phenyl, benzyl, pyridyl, —OH, —NH₂, —F, —Cl, —Br, —I, —NO₂ or —CN. In certain embodiments substituents for a cycloalkyl group, such as cycloalkyl groups that may be present at positions represented by R₁ and R₂, are alkyl groups, such as a methyl or ethyl group. In certain embodiments R₁ and R₂ are both a C3-C8 cycloalkyl group optionally substituted with at least one alkyl group.

In some embodiments the bis(thio-hydrazamide amide) is any of Compounds (1)-(18) as described in U.S. Patent Application Publication No. 20080119440.

In some embodiments the bis(thio-hydrazide amide) is elesclomol, the structure of which is as follows:

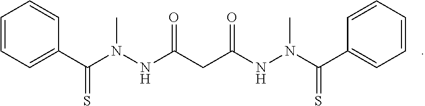 [SMILES: CN(NC(=O)CC(=O)NN(C)C(=S)c1ccccc1)C(=S)c1ccccc1]

In some embodiments the bis(thio-hydrazide amide) is eleselomol or an analog thereof Some examples of suitable analogs are as follows:

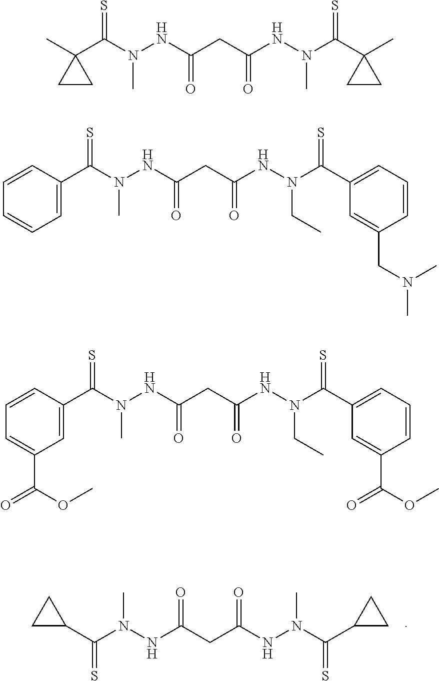

In some aspects, any of the bis(thio-hydrazide amide) compounds described herein (e.g., elesclomol or an analog thereof) is used conjointly with any of proteasome inhibitors (e.g., bortezomib, carfilzomib, opmzomib, ixazoinib, delanzomib, or an analog of any of these) to treat a subject in need of treatment for cancer. The cancer may be resistant to the proteasome inhibitor. In some embodiments the bis(thio-hydrazide amide) compounds described herein (e.g., elesclomol or an analog thereof) and proteasome inhibitor are administered in the same composition. In some embodiments they are administered separately. In some embodiments a method comprises administering a bis(thio-hydrazide amide) to a subject who has received or is expected to receive one or more doses of a proteasome inhibitor. A subject who is expected to receive a proteasome inhibitor may be one to whom a proteasome inhibitor has been prescribed or for whom a plan to prescribe or administer a proteasome inhibitor has been committed to a tangible medium by a health care provider of the subject, e.g., the subject's oncologist. In some embodiments the subject is expected to receive the proteasome inhibitor within 4 weeks of administration of the bis(thio-hydrazide amide). In some embodiments a method comprises administering a proteasome inhibitor to a subject who has received or is expected to receive one or more doses of a bis(thio-hydrazide amide). A subject who is expected to receive a bis(thio-hydrazide amide) may be one to whom a bis(thio-hydrazide amide) has been prescribed or for whom a plan to prescribe or administer a bis(thio-hydrazide amide) has been committed to a tangible medium by a health care provider of the subject, e.g., the subject's oncologist. In some embodiments the subject is expected to receive the bis(thio-hydrazide amide) within 4 weeks of administration of the proteasome inhibitor.

In some embodiments, any of the bis(thio-hydrazide amide) compounds described herein (e.g., elesclomol or an analog thereof) may be used conjontly with any one or more EGFR inhibitors (e.g., Gefitinib, Osimertinib, tyrosine kinase inhibitors, or an analog of any of these) to treat a subject in need of treatment for cancer. The cancer may be resistant to the EGFR inhibitor. In some embodiments, the bis(thio-hydrazide amide) compounds described herein (e.g., elesclomol or an analog thereof) and EGFR inhibitor are administered in the same composition. In other embodiments, they are administered separately. In some embodiments, a method comprises administering a bis(thio-hydrazide amide) to a subject who has received or is expected to receive one or more doses of a EGFR inhibitor, e.g., a subject to whom a EGFR inhibitor has been prescribed or for whom a plan to prescribe or administer a EGFR inhibitor has been committed to a tangible medium by a health care provider of the subject, e.g., the subject's oncologist. In some such embodiments, the subject may be expected to receive the EGFR inhibitor within 4 weeks of administration of the bis(thio-hydrazide amide).

In some embodiments, the method comprises administering a EGFR inhibitor to a subject who has received or is expected to receive one or more doses of a bis(thio-hydrazide amide), e.g., a subject to whom a bis(thio-hydrazide amide) has been prescribed or for whom a plan to prescribe or administer a bis(thio-hydrazide amide) has been committed to a tangible medium by a health care provider of the subject, e.g., the subject's oncologist. In some such embodiments, the subject is expected to receive the bis(thio-hydrazide amide) within 4 weeks of administration of the EGFR inhibitor.

In some aspects, it is contemplated to use elesclomol analogs that contain a single C═S moiety for any of the purposes described herein for elesclomol. For example, one of the C═S moieties in elesclomol or other bis(thiohydrazide amides) of Formula A or B above may be replaced by a C═O moiety. For example, in some embodiments the compound is the following:

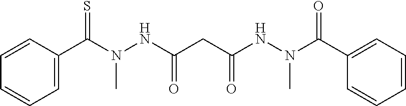 [SMILES: CN(NC(=O)CC(=O)NN(C)C(=S)c1ccccc1)C(=O)c1ccccc1]

In some embodiments it is contemplated to use compounds of formula (I) as presented in US Patent Application Publication No. 20120065206 (US20120065206) for any of the purposes for which elesclomol (or other bis(thio-hydrazide amide)) may be used as described herein. Such compounds are considered elesclomol analogs for purposes of the present disclosure. Such compounds, which may be referred to as sulfonylhydrazide compounds, are depicted as follows:

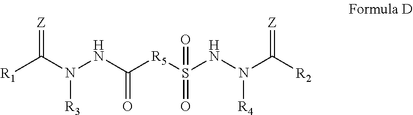

wherein each Z is independently S, O or Se, provided that Z cannot both be O; R₁ and R₂, are each independently selected from the group consisting of an optionally substituted alkyl, an optionally substituted alkenyl, an optionally substituted alkynyl; an optionally substituted cycloalkyl, an optionally substituted cycloalkenyl, an optionally substituted heterocyclic group wherein the heterocyclic group is bonded to the thiocarbonyl carbon via a carbon-carbon linkage, an optionally substituted phenyl, an optionally substituted bicyclic aryl, an optionally substituted five to seven-membered monocyclic heteroaryl, an optionally substituted nine to fourteen-membered bicyclic heteroaryl wherein the heteroaryl group is bonded to the thiocarbonyl carbon via a carbon-carbon linkage, —NR₁₂R₁₃, OR₁₄, —SR₁₄ and —S(O)ₚR₁₅; R₃ and R₄ are each independently selected from the group consisting of hydrogen, an optionally substituted alkyl, an optionally substituted alkenyl, an optionally substituted alkynyl, an optionally substituted cycloalkyl, an optionally substituted cycloalkenyl, an optionally substituted heterocyclic group, and an optionally substituted five to six-membered aryl or heteroaryl group; or R₁ and R₃ and/or R₂ and R₄, taken together with the atoms to which they are attached, form an optionally substituted heterocyclic group or an optionally substituted heteroaryl group; R₅ is —CR₆R₇—, —C(═CHR₈)— or —C(═NR8)-; R₆ and R₇ are both —H or an optionally substituted lower alkyl; R8 is selected from the group consisting of —OH, an alkyl, an alkenyl, an alkynyl, an alkoxy, an alkenoxy, an alkynoxyl, a hydroxyalkyl, a hydroxyalkenyl, a hydroxyalkynyl, a haloalkyl, a haloalkenyl, a haloalkynyl, an optionally substituted phenyl, an optionally substituted bicyclic aryl, an optionally substituted five to six-membered monocyclic heteroaryl, an optionally substituted nine to fourteen-membered bicyclic heteroaryl, an optionally substituted cycloalkyl or an optionally substituted heterocyclic group; —NR₁₀R₁₁, and —COR₉; R₉ is an optionally substituted phenyl, an optionally substituted bicyclic aryl, an optionally substituted five or six-membered monocyclic heteroaryl, an optionally substituted nine to fourteen-membered bicyclic heteroaryl, an optionally substituted alkyl, an optionally substituted cycloalkyl or an optionally substituted heterocyclic group; R₁₀ and R₁₁ are each independently selected from the group consisting of —H, —OH, amino, (di)alkylamino, an alkyl, an alkenyl, an alkynyl, an alkoxy, an alkenoxy, an alkynoxyl, a hydroxyalkyl, a hydroxyalkenyl, a hydroxyalkynyl, a haloalkyl, a haloalkenyl, a haloalkynyl, an optionally substituted phenyl, an optionally substituted bicyclic aryl, an optionally substituted five to six-membered monocyclic heteroaryl, an optionally substituted nine to fourteen-membered bicyclic heteroaryl, an optionally substituted cycloalkyl or an optionally substituted heterocyclic group and —COR₉, or R₁₀ and R₁₁, taken together with the nitrogen atom to which they are attached, form a five to six-membered heteroaryl group; and R₁₂, R₁₃ and R₁₄ are each independently —H, an optionally substituted alkyl, an optionally substituted phenyl or an optionally substituted benzyl, or R₁₂ and R₁₃, taken together with the nitrogen atom to which they are attached, form an optionally substituted heterocyclic group or an optionally substituted heteroaryl group; R₁₅ is an optionally substituted alkyl, an optionally substituted aryl or an optionally substituted heteroaryl, and p is 1 or 2; provided that when both Z are S and R₃ and R₄ are both methyl, then R₁ and R₂ are not both unsubstituted phenyl. In some embodiments R₁₀ and R₁₁ are not both —H. It is contemplated in certain embodiments to use compounds of Formula D above, wherein both Z are S and R₃ and R₄ are both methyl and R1 and R2 are both unsubstituted phenyl. In certain embodiments of formula D at least one Z is S. In certain embodiments of Formula D, both Z are S. In some embodiments it is contemplated to use compounds of Formula D, wherein both Z are S and R₃ and R₄ are methyl and R₁ and R₂ are both lower alkyl, e.g., cyclopropyl or methylcyclopropyl.

In certain embodiments the compound may be any of compounds 1-91 depicted in US 20120065206.

In some embodiments it is contemplated to use compounds of Formula D, wherein Z, R₁, R₂, R₃, R₄, R₇, and R₈ are defined as for formula A above, for any of the purposes for which elesclomol (or other bis(thio-hydrazide amide)) may be used as described herein. In certain embodiments the compound is of the following formula:

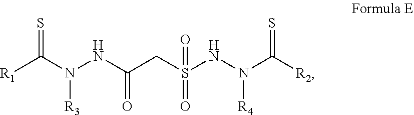

wherein R₁, R₂, R₃, and R₄ are as defined above for Formula A or D. In certain embodiments R₁, R₂, or both, are phenyl or lower alkyl, e.g., methyl, propyl, cyclopropyl or methylcyclopropyl. In certain embodiments R₃, R₄, or both, are lower alkyl, e.g., methyl. In some embodiments R₁ and R₂ are the same. In certain embodiments R₃ and R₄ are the same. In some embodiments the compound has the following structure:

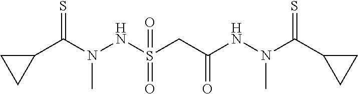 [SMILES: CN(NC(=O)CS(=O)(=O)NN(C)C(=S)C1CC1)C(=S)C1CC1]

In some embodiments it is contemplated to use compounds of formulae (I), (III), (IV), (VII), (X), (XI), (XII), (XIII) or (XIV) as presented in US Patent Application Publication No. 20150025042 for any of the purposes for which elesclomol (or other bis(thio-hydrazide amide)) may be used as described herein. In some embodiments the compound comprises at least one C═S moiety. In some embodiments the compound comprises two C═S moiety In certain embodiments the compound is of the following formula:

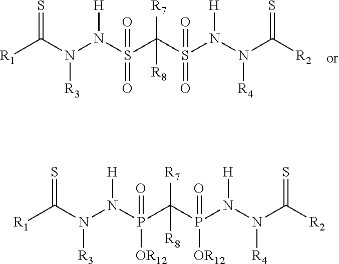

wherein R₁, R₂, R₃, R₄, R₇, R₈, and R₁₂ are as defined in US Patent Application Publication No. 20150025042.

It should be understood that where the disclosure refers to compounds disclosed in a particular publication (e.g., a patent, patent application, journal article, etc.), such compounds include each of the various genera, subgenera, and species disclosed in such reference.

In some embodiments a compound that selectively inhibits growth of cancer cells is a compound capable of forming a complex with copper, Cu(II). Without wishing to be bound by any theory, the copper-agent complex may generate copper-mediated oxidative stress. In some embodiments, the compound that is capable of forming a complex with copper is a bis(thiohydrazide) amide or dithiocarbamate. In some embodiments the compound is additionally or alternately capable of forming a complex with zinc.

In some embodiments, a compound that selectively inhibits growth of cancer cells is an agent that causes an increased level of one or more reactive oxygen species (ROS) in cells with which it is contacted. ROS are chemically reactive molecules containing oxygen. Exemplary ROS are peroxides (e.g., hydrogen peroxides), superoxide, hydroxyl radical, and singlet oxygen. A compound that causes an increased level of one or more ROS may be referred to as “ROS inducer”, A ROS inducer may, for example, inhibit an enzyme or biological pathway or process that would normally be responsible for reducing ROS (e.g., converting a ROS into a less reactive species) or may activate an enzyme or biological pathway or process that increases ROS in cells. Increased levels of ROS often result in, among other things, lipid peroxidation, which can generate numerous aldehyde species that are toxic to cells. In some embodiments a compound that selectively inhibits growth of cancer cells is an agent that is an oxidative stress promoting agent. The term “oxidative stress promoting agent” refers to ROS inducers and agents that impair the ability of a cell or organism to metabolize, inhibit, or remove harmful species that are generated as a result of ROS. For example, an oxidative stress promoting agent may inhibit an enzyme such as aldehyde dehydrogenase (ALDH) that would normally be responsible for converting a reactive protein or lipid species that has been generated through oxidation by ROS into a less reactive form.

In some embodiments an ROS inducer is a dithiocarbamate (e.g., disulfiram or an analog or active metabolite thereof) or a bis(thio-hydrazide amide) (e.g., elesclomol or an analog or active metabolite thereof). In some embodiments a ROS inducer is a metal such as iron, copper, chromium, vanadium, and cobalt that is capable of redox cycling in which a single electron may be accepted or donated by the metal. This action catalyzes production of reactive radicals and reactive oxygen species. In some embodiments a ROS inducer is a compound that forms a complex with such a metal.

In some embodiments, a compound that selectively inhibits growth of cancer cells is an aldehyde dehydrogenase (ALDH) inhibitor. Aldehyde dehydrogenases catalyze the irreversible oxidation of aldehydes to their corresponding carboxylic acid, thereby protecting cells from aldehyde-induced cytotoxicity. The human ALDH superfamily comprises 19 ALDH polypeptides: ALDH1A1, ALDH1A2, ALDH1A3, ALDH1B1, ALDH1L1, ALDH1L2, ALDH2, ALDH3A1, ALDH3A2, ALDH3B1, ALDH3B2, ALDH4A1, ALDH5A1, ALDH6A1, ALDH7A1, ALDH8A1, ALDH9A1, ALDH16A1, and ALDH18A1. These enzymes catalyze the oxidation of an aldehyde (e.g., an endogenously produced aldehyde such as those generated during metabolism or an exogenous aldehyde) to its respective carboxylic acid in an NAD⁺-dependent or NADP⁺-dependent reaction. Exemplary amino acid sequences of ALDH polypeptides (e.g., human sequences) and nucleic acids that encode them are known in the art and available in public databases such as the NCBI RefSeq database,

“ALDH inhibitor” refers to an agent that inhibits expression or activity of at least one member of the ALDH superfamily. In some embodiments of any of the methods or compositions described herein relating to ALDH inhibitors, the ALDH inhibitor inhibits the expression and/or activity of one or more of ALDH1A1, ALDH1A2, ALDH1A3, ALDH1B1, ALDH1L1, ALDH1L2, ALDH2, ALDH3A1, ALDH3A2, ALDH3B1, ALDH3B2, ALDH4A1, ALDH5A1, ALDH6A1 ALDH7A1, ALDH8A1, ALDH9A1, ALDH16A1, and ALDH18A1. In some embodiments, an ALDH inhibitor inhibits the expression and/or activity of one or more members of the ALDH1 family (ALDH1A1, ALDH 1A2, ALDH1A3, ALDH1B1, ALDH1L1, and ALDH1L2). In some embodiments, an ALDH inhibitor inhibits the expression and/or activity of at least ALDH1A1. In some embodiments, an ALDH inhibitor inhibits the expression and/or activity of at least ALDH1 A2. In some embodiments an ALDH inhibitor inhibits the expression and/or activity of ALDH2. In some embodiments an ALDH inhibitor inhibits the expression and/or activity of one or more members of the ALDH3 family (ALDH3A1, ALDH3A2, ALDH3B1, and ALDH3B2). In some embodiments an ALDH inhibitor inhibits the expression and/or activity of ALDH4A1, ALDH5A1, ALDH6A1, ALDH7A1, ALDH8A1, ALDH9A1, ALDH16A1, and/or ALDH18A1. In some embodiments, an ALDH inhibitor inhibits the expression and/or activity of one or more members of the ALDH1 family and ALDH2.

An ALDH inhibitor may comprise a small molecule, nucleic acid (e.g., siRNA, aptamer), or protein (e.g., an antibody or non-antibody polypeptide). In some embodiments, the ALDH inhibitor binds to an ALDH polypeptide and inhibits its activity. In some embodiments the binding is reversible. In some embodiments a stable covalent bond between the ALDH inhibitor and ALDH is formed. For example, a covalent bond to an amino acid in the active site of the enzyme (e.g., Cys302) may be formed. In some embodiments the ALDH inhibitor is metabolized to one or more active metabolite(s) that at least in part mediate its inhibitory activity. Any of a wide variety of ALDH inhibitors are known in the art and may be used in compositions and methods described herein. Further information regarding ALDHs and certain ALDH inhibitors is found in Koppaka, V., et al., Pharmacological Reviews, (2012) 64: 520-539.

In certain embodiments an ALDH inhibitor is a dithiocarbamate, e.g., disulfiram, or an analog or metabolite of a dithiocarbamate, e.g., a disulfiram metabolite. Disulfiram inhibits ALDH1A1 and ALDH2. Disulfiram metabolites that are ALDH inhibitors include, e.g., NN-diethyldithiocarbamate, S-methyl NN-diethyldithiocarbamate, S-methyl N,N-diethyldithiocarbamate sulfoxide, S-methyl NN-diethylthiocarbamate sulfoxide, S-methyl NN-diethyldithiocarbamate sulfone, and S-methyl NN-diethylthiocarbamate sulfone. Disulfiram and certain other ALDH inhibitors are used clinically in the treatment of alcoholism. Alcohol consumption by patients being treated with disulfiram results in acetaldehyde accumulation, leading to a number of unpleasant symptoms that discourage the patient from consuming alcohol. Disulfiram is also an inhibitor of dopamine-β-hydroxylase and has use in treating cocaine addiction.

In some embodiments an ALDH inhibitor is a quinazolinone derivative described in US Pat. App. Pub. No. 20080249116 of the following formula, where R¹, R², R³, W, and V are as described therein:

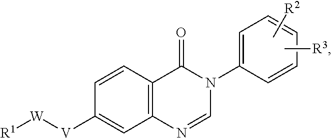

In some embodiments an ALDH inhibitor is a compound described in US Pat. Pub. No. 20040068003 of the following formula, wherein R1, R2, R3, R4, R5, R6, and R7 are as described therein:

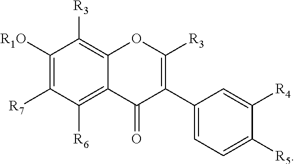

In some embodiments an ALDH inhibitor is a compound of the formula:

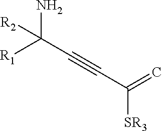

wherein R₁, R₂ and R₃, independently represent a substituted or substituted linear or branched C₁-C₆ alkyl radical, or a salt thereof.

In some embodiments an ALDH inhibitor is a compound described in PCT/US2014/067943 (WO/2015/084731), entitled ALDEHYDE DEHYDROGENASE INHIBITORS AND METHODS OF USE THEREOF). In some embodiments the compound is of the following Formula I:

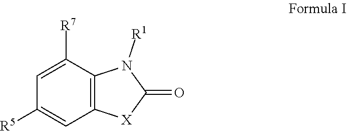

wherein X is O or —C═O; R¹ is H, alkyl, substituted alkyl, alkenyl, substituted alkenyl, alkynyl, substituted alkynyl, cycloalkyl, substituted cycloalkyl, heterocycloalkyl, substituted heterocycloalkyl, aryl, substituted aryl, heteroaryl or substituted heteroaryl; R⁵ is H, alkyl, substituted alkyl, halo, alkoxy or substituted alkoxy; and R⁷ is H or halo.

In some embodiments the compound is of the following Formula II:

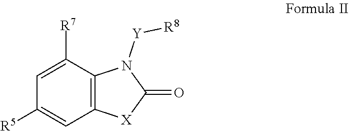

wherein X is O or —C═O; Y is alkyl, substituted alkyl, alkenyl, substituted alkenyl, alkynyl or substituted alkynyl; R⁵ is alkyl, substituted alkyl, halo, alkoxy or substituted alkoxy; R⁷ is H or halo; and R⁸ is cycloalkyl, substituted cycloalkyl, heterocycloalkyl, substituted heterocycloalkyl, aryl, substituted aryl, heteroaryl or substituted heteroaryl.

In some embodiments the compound is of the following Formula III:

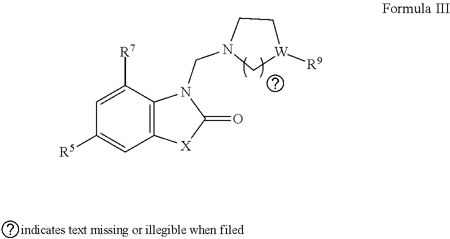

wherein n is 1 or 2; X is O or —C═O; W is N or O, and when W is O, then R⁹ is not present; R⁵ is H, alkyl, substituted alkyl, halo, alkoxy or substituted alkoxy; le is ET or halo; R⁹ is H or —(CH₂)ₘR¹⁰, where m is an integer from 1 to 6; and R¹0 is H, cycloalkyl, substituted cycloalkyl, heterocycloalkyl, substituted heterocycloalkyl, aryl, substituted aryl, heteroaryl or substituted heteroaryl.

Other ALDH inhibitors include coprine, cyanamide, 1-aminocyclopropanol (ACP), daidzin (i.e., the 7-glucoside of 4′,7-dihydroxyisoflavone), CVT-10216 (3-[[[3 [(Methylsulfonyl)amino]phenyl]-4-oxo-4H-1-benzopyran-7-yl]oxy]methyl]benzoic acid; CAS Registry number 1005334-57-5), cephalosporins, antidiabetic sulfonylureas, metronidazole, diethyldithiocarbamate, phenethyl isothiocyanate (PEITC), prunetin (4%5-dihydroxy-7-methoxyisoflavone), 5-hydroxydaidzein (genistein), trichloroacetaldehyde monohydrate (or chloral), 4-amino-4-methyl-2-pentynethioic acid (S)-methyl ester. In some embodiments an ALDH inhibitor comprises 4-amino-4-methyl-2-pentyne-1-al (AMPAL) or 2-methyl-5-(methylsulfanyl)-5-oxopentan-2-aminium, which are irreversible inhibitors of the ALDH1 and ALDH3 enzymes. In some embodiments an ALDH inhibitor comprises benomyl (methyl-[1-[(butylamino)carbonyl]-1H-benzimidazol-2-yl]carbamate). In some embodiments, an ALDH inhibitor is an oral hypoglycemic agent such as chlorpropamide or tolbutamide. In some embodiments an ALDH inhibitor is gossypol or an analog thereof. In some embodiments, an ALDH inhibitor is 2,2′-bis-(formyl-1,6,7-trihydroxy-5-isopropyl-3-methylnaphthalene). In some embodiments an ALDH inhibitor is a compound with any of the following CAS Registry numbers: 1069117-57-2, 1069117-56-1, 10691 17-55-0, 1055417-23-6, 1055417-22-5, 1055417-21-4, 1055417-20-3, 1055417-19-0, 1055417-18-9, 1055417-17-8, 1055417-16-7, 1055417-15-6 and 1055417-13-4.

In some embodiments an ALDH inhibitor is an aromatic lactone described in Buchman, C D, et al, Chemico-Biological Interactions (2015) 234:38-44.

In some embodiments an ALDH inhibitor comprises a nucleic acid that inhibits ALDH gene expression or activity. In some embodiments the nucleic acid is an RNA (agent (e.g., an siRNA) that inhibits ALDH gene expression. Exemplary nucleic acid ALDH inhibitors and formulations comprising them are described in US Pat. Pub. No. 20140248338.

In some embodiments an ALDH inhibitor is selective for one or more ALDH enzymes as compared to one or more other ALDH enzymes. As used herein, an inhibitor is considered selective for a first enzyme as compared to a second enzyme if the IC50 of the agent for the first enzyme is at least 5-fold lower than the IC50 of the agent for the second enzyme. In some embodiments the difference in IC50 values is at least 10-fold, at least 100-fold, or at least 1000-fold. In some embodiments an ALDH inhibitor is selective for one or more ALDH1 family members (e.g., ALDH1A1) as compared to ALDH2. In some embodiments an ALDH inhibitor is selective for one or more ALDH1 family members (e.g., ALDH1A1) and for ALDH2 as compared to at least some of the other ALDH superfamily members (e.g., ALDH3A1). In some embodiments an ALDH inhibitor is selective for one or more ALDH enzymes as compared with other dehydrogenases such as 15-hydroxyprostaglandin dehydrogenase (HPGD) and type 4hydroxysteroid dehydrogenase (HSD17β4) HPGD and HSD17β4,

In some embodiments of any of the compositions or methods described herein that relate to an ALDH inhibitor, the ALDH inhibitor binds to at least one ALDH superfamily member with a Kd of ≤100 nM, e.g., 50 nM-100 nM. In some embodiments the ALDH inhibitor binds to at least one ALDH polypeptide with a Kd of ≤50 nM, e.g., 10 nM-50 nM. In some embodiments the ALDH inhibitor binds to at least one ALDH superfamily member with a Kd of ≤10 nM, e.g., 1 nM-10 nM. In some embodiments the ALDH inhibitor binds to at least one ALDH superfamily member with a Kd of ≤1 nM, e.g., 0.1 nM to 1 nM or 0.01 nM to 0.1 nM. In some embodiments the ALDH inhibitor binds to at least one ALDH1 family member with a Kd of ≤100 nM, e.g., 50 nM-100 nM. In some embodiments the ALDH inhibitor binds to at least one ALDH1 family member with a Kd of ≤50 nM, e.g., 10 nM-50 nM. In some embodiments the ALDH inhibitor binds to at least one ALDH1 family member with a Kd of ≤10 nM, e.g., 1 nM-10 nM. In some embodiments the ALDH inhibitor binds to at least one ALDH1 family member with Kd of ≤1 nM, e.g., 0.1 nM to 1 nM or 0.01 nM to 0.1 nM. In some embodiments the ALDH inhibitor binds to ALDH2 with a Kd of ≤100 nM, e.g., 50 nM-100 nM. In some embodiments the ALDH inhibitor binds to ALDH2 with a Kd of ≤50 nM, e.g., 10 nM-50 nM. In some embodiments the ALDH inhibitor binds to ALDH2 with a Kd of ≤10 nM, e.g., 1 nM-10 nM. In some embodiments the ALDH inhibitor binds to ALDH2 with a Kd of ≤1 nM, e.g., 0.1 nM to 1 nM or 0.01 nM to 0.1 nM.

In some embodiments of any of the compositions or methods described herein that relate to a ALDH inhibitor, the ALDH inhibitor inhibits one or more ALDH polypeptides with an IC50 of 1 nM-5 μM, e.g., 1 nM-5 nM, 5 nM-10 nM, 10 nM-20 nM, 20 nM-30 nM, 30 nM-50 nM, 50 nM-100 nM, 100 nM-500 nM, 500 nM-1μ, or 1-5 μM. In some embodiments the ALDH inhibitor inhibits one or more ALDH1 polypeptides with an IC50 of 1 nM-5 μM, e.g., 1 nM-5 nM, 5 nM-10 nM, 10 nM-20 nM, 20 nM-30 nM, 30 nM-50 nM, 50 nM-100 nM, 100 nM-500 nM, 500 nM-1 μM, or 1 μM-5 μM. In some embodiments the ALDH inhibitor inhibits ALDH1A1 with an IC50 of 1 nM-5 μM, e.g., 1 nM-5 nM, 5 nM-10 nM, 10 nM-20 nM, 20 nM-30 nM, 30 nM-50 nM, 50 nM-100 nM, 100 nM-500 nM, 500 nM-1 μM, or 1 μM-5 μM. In some embodiments the ALDH inhibitor inhibits ALDH1A2 with an IC50 of 1 nM-5 μM, e.g., 1 nM-5 nM, 5 nM-10 nM, 10 nM-20 nM, 20 nM-30 nM, 30 nM-50 nM, 50 nM-100 nM, 100 nM-500 nM, 500 nM-1 μM, or 1 μM-5 μM. In some embodiments the ALDH inhibitor inhibits ALDH2 with an IC50 of 1 nM-5 μM, e.g., 1 nM-5 nM, 5 nM-10 nM, 10 nM-20 nM, 20 nM-30 nM, 30 nM-50 nM, 50 nM-100 nM, 100 nM-500 nM, 500 nM-1 μM, or 1 μM-5 μM.

### Methods of Inhibiting Tumor Growth and Proliferation

In certain aspects, provided herein are methods related to inhibiting tumor growth and/or proliferation that comprise determining a level of a biomarker in a tumor sample comprising tumor cells and contacting the tumor with a therapeutic compound if at least a threshold portion of the sample has a level of the biomarker. In certain embodiments, the therapeutic compound used to contact the tumor is a copper ionophore. In certain embodiments, the biomarker is a lipoylated protein. Exemplary lipoylated protein biomarkers are listed in Table 2. In certain embodiments, the biomarker is a mitochondrial protein. In certain embodiments the mitochondrial protein is involved in lipoic acid biosynthesis. In certain embodiments, the mitochondrial protein is an iron-sulfur cluster protein. In certain embodiments the mitochondrial protein is a mitochondrial Complex I protein. Exemplary mitochondrial genes encoding relevant mitochondrial protein biomarkers are listed in Table 3.

TABLE 2
Exemplary Lipoylated Protein Biomarkers

| Lipoylated Protein | Function |
|---|---|
| Lipoyl-DLAT | Pyruvate dehydrogenase |
| | complex |
| Lipoyl-DLST | Alpha-keto glutarate |
| | dehydrogenase complex |
| Lipoyl-GCSH | Glycine cleave system |
| Lipoyl-DBT | Branched-chain alpha-keto |
| | acid dehydrogenase complex |

TABLE 3
Exemplary Mitochondrial Genes of Relevant
Mitochondrial Protein Biomarkers

| Gene Name | Function |
|---|---|
| FDX1 | Fe—S cluster pathway |
| ALDHA1 | Oxidation of aldehydes |
| ALDH2 | Oxidation of aldehydes |
| LIAS | Lipoic acid pathway |
| LIPT1 | Lipoic acid pathway |
| LIPT2 | Lipoic acid pathway |
| DLD | Lipoic acid pathway |
| NDUFB6 | Complex I |
| NDUFC2 | Complex I |
| NDUFA6 | Complex I |
| NDUFS1 | Complex I |
| ISCA2 | Fe—S cluster pathway |
| PDHB | Pyruvate dehydrogenase |
| NDUFS8 | Complex I |
| NDUFA2 | Complex I |
| NDUFS3 | Complex I |
| NDUFA9 | Complex I |
| NDUFV1 | Complex I |
| NDUFS2 | Complex I |
| NDUFB8 | Complex I |
| NDUFV2 | Complex I |
| NDUFB11 | Complex I |
| NDUFC1 | Complex I |
| CNGA2 | Cyclic nucleotide-gated |
| | ion channel |
| PLOD1 | Hydroxylation of lysine |
| ST6GAL2 | Sialyltransferase |
| ABCA13 | ATP Binding Cassette |
| GLRX5 | Fe—S Cluster pathway |

In certain embodiments, the method includes determining a level of protein lipoylation (e.g. lipoyl-DLAT (lipoyl-dihydrolipoamide acetyltransferase), lipoyl-DLST (lipoyl-dihydrolipoyl succinyltransferase), lipoyl-GCSH (lipoyl-Glycine Cleavage System Protein H), lipoyl-DBT (lipoyl-dihydrolipoamide branched chain transacylase E2), or any combination thereof) in the tumor sample and contacting the tumor with a therapeutic compound if the level of protein lipoylation in the sample is above a threshold level. In certain embodiments, the method includes determining a level of protein lipoylation (e.g. lipoyl-DLAT, lipoyl-DLST, lipoyl-GCSH, lipoyl-DBT, or any combination thereof) and determining a level of mitochondria protein expression (e.g. FDX1 (ferredoxin 1), ALDHA1 (aldehyde dehydrogenase A1), ALDH2 (aldehyde dehydrogenase 2), LIAS (lipoic acid synthetase), LIPT1 (lipoyltransferase 1), LIPT2 (lipoyltransferase 2), DLD (Dihydrolipoamide Dehydrogenase) (or any combination thereof) in the tumor sample and contacting the tumor with a therapeutic compound if the level of protein lipoylation in the sample is above a threshold level and if the level of mitochondrial protein expression in the sample is above a threshold level.

In certain embodiments, the threshold level of the total tumor cells expressing lipoyl-DLAT in a sample is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% of the sample express lipoyl-DLAT.

In certain embodiments, the threshold level of the total tumor cells expressing lipoyl-DLST in a sample is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the sample express lipoyl-DLST.

In certain embodiments, the threshold level of the total tumor cells expressing lipoyl-GCSH in a sample is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the sample express lipoyl-GCSH.

In certain embodiments, the threshold level of the total tumor cells expressing lipoyl-DBT in a sample is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the sample express lipoyl-DBT.

In certain embodiments, the threshold level of the total tumor cells expressing FDX1 in a sample is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the sample express FDX1.

In certain embodiments, the threshold level of the total tumor cells expressing ALDHA1 in a sample is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the sample express ALDHA1.

In certain embodiments, the threshold level of the total tumor cells expressing ALDH2 in a sample is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the sample express ALDH2.

In certain embodiments, the threshold level of the total tumor cells expressing LIAS in a sample is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the sample express LIAS.

In certain embodiments, the threshold level of the total tumor cells expressing LIPT1 in a sample is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the sample express LIPT1.

In certain embodiments, the threshold level of the total tumor cells expressing LIPT2 in a sample is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the sample express LIPT2.

In certain embodiments, the threshold level of the total tumor cells expressing DLD in a sample is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the sample express DLD.

In some embodiments, any assay capable of detecting expression of the relevant biomarker can be used in the methods provided herein. In some embodiments, the biomarker is detected by immunostaining with a labeled antibody that binds to the biomarker epitope. In some embodiments, the biomarker is detected by immunohistochemistry. In some embodiments, the biomarker is detected by Western Blot. In some embodiments, the mRNAs of the biomarker are detected using qPCR. In some embodiments, the biomarker is detected using fluorescence activated cell sorting (FACS). In some embodiments, the biomarker is detected using microscopy (e.g., fluorescence microscopy). In some embodiments, the biomarker is detected using ELISA.

Any of a variety of antibodies can be used in methods of the detection. Such antibodies include, for example, polyclonal, monoclonal (mAbs), recombinant, humanized or partially humanized, single chain, Fab, and fragments thereof. The antibodies can be of any isotype, e.g., IgM, various IgG isotypes such as IgG1, IgG2a, etc., and they can be from any animal species that produces antibodies, including goat, rabbit, mouse, chicken or the like. The term “an antibody specific for” a protein means that the antibody recognizes a defined sequence of amino acids, or epitope, in the protein, and binds selectively to the protein and not generally to proteins unintended for binding to the antibody. The parameters required to achieve specific binding can be determined routinely, using conventional methods in the art.

In some embodiments, antibodies specific for a biomarker (e.g., lipoyl-DLAT, lipoyl-DLST, lipoyl-GCSH, lipoyl-DBT, FDX1, ALDHA1, ALDH2, LIAS, LIPT1, LIPT2, DLD) are immobilized on a surface (e.g., are reactive elements on an array, such as a microarray, or are on another surface, such as used for surface plasmon resonance (SPR)-based technology, such as Biacore), and proteins in a sample are detected by virtue of their ability to bind specifically to the antibodies. Alternatively, proteins in the sample can be immobilized on a surface, and detected by virtue of their ability to bind specifically to the antibodies. Methods of preparing the surfaces and performing the analyses, including conditions effective for specific binding, are conventional and well-known in the art.

Among the many types of suitable immunoassays are immunohistochemical staining, ELISA, Western blot (immunoblot), immunoprecipitation, radioimmunoassay (MA), fluorescence-activated cell sorting (FACS), etc. In some embodiments, assays used in methods provided herein can be based on colorimetric readouts, fluorescent readouts, mass spectroscopy, visual inspection, etc.

As mentioned above, expression levels of a biomarker can be measured by measuring nucleic acid amounts (e.g., mRNA amounts and/or genomic DNA). The determination of nucleic acid amounts can be performed by a variety of techniques known to the skilled practitioner. For example, expression levels of nucleic acids, alternative splicing variants, chromosome rearrangement and gene copy numbers can be determined by microarray analysis (see, e.g., U.S. Pat. Nos. 6,913,879, 7,364,848, 7,378,245, 6,893,837 and 6,004,755) and quantitative PCR. Copy number changes may be detected, for example, with the Illumina Infinium II whole genome genotyping assay or Agilent Human Genome CGH Microarray (Steemers et al., 2006). Examples of methods to measure mRNA amounts include reverse transcriptase-polymerase chain reaction (RT-PCR), including real time PCR, microarray analysis, nanostring, Northern blot analysis, differential hybridization, and ribonuclease protection assay. Such methods are well-known in the art and are described in, for example, Sambrook et al., Molecular Cloning: A Laboratory Manual, current edition, Cold Spring Harbor Laboratory, Cold Spring Harbor, N.Y., and Ausubel et al., Current Protocols in Molecular Biology, John Wiley & sons, New York, N.Y.

### Methods of Treating Cancer

In certain embodiments, the provided herein are methods of treating a cancer in a subject by administering to the subject a therapeutic compound according to a method provided herein. In some embodiments, the therapeutic compound is a copper ionophore. In some embodiments, the methods described herein may be used to treat any cancerous, pre-cancerous tumor, and/or immune cells. In some embodiments, contacting the tumor and/or immune cell with the copper ionophore inhibits Pyruvate dehydrogenase complex, 2-oxoglutarate dehydrogenase complex, Branched-Chain Alpha-Keto Acid Dehydrogenase Complex, and/or glycine cleavage. In some embodiments, the copper ionophore is pre-loaded (e.g., pre-complexed) with copper(II).

In some embodiments, the cancer includes a solid tumor. Cancers that may be treated by methods and compositions provided herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Actual dosage levels of the therapeutic compound may be varied so as to obtain an amount which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

In certain embodiments, the therapeutic compound disclosed herein can be conjointly administered with an anti-cancer agent, e.g., chemotherapeutic agents, immune checkpoint inhibitors, and/or proteasome inhibitors. The treatment methods can be administered in conjunction with other forms of conventional therapy (e.g., standard-of-care treatments for cancer well known to the skilled artisan), either consecutively with, pre- or post-conventional therapy. For example, these modulatory agents can be administered with a therapeutically effective dose of chemotherapeutic agent. In another embodiment, these modulatory agents are administered in conjunction with chemotherapy to enhance the activity and efficacy of the chemotherapeutic agent. In certain aspects disclosed herein, the therapeutic compound can be conjointly administered with an immune checkpoint inhibitor. Checkpoint inhibitor therapies target key regulators of the immune system that either stimulate or inhibit the immune response. Such immune checkpoints can be exploited in the cancer disease state (e.g., by tumors) to evade attacks by the immune system. In certain aspects disclosed herein, the therapeutic compound can be conjointly administered with a proteasome inhibitor.

In some embodiments, the method of treating or preventing cancer (e.g., breast cancer, lung cancer, such as non-small cell lung cancer, prostate cancer, colon cancer, bladder cancer, gastric cancer, ovarian cancer, melanoma, and renal cancer) may comprise administering a compound conjointly with one or more other chemotherapeutic agent(s). Chemotherapeutic agents that may be conjointly administered with therapeutic compounds include: ABT-263, afatinib dimaleate, aminoglutethimide, amsacrine, anastrozole, asparaginase, axitinib, b-raf inhibitors (e.g., vemurafenib, dabrafenib), Bacillus Calmette-Guérin vaccine (bcg), bevacizumab, BEZ235, bicalutamide, bleomycin, bortezomib, buserelin, busulfan, cabozantinib, campothecin, capecitabine, carboplatin, carfilzomib, carmustine, ceritinib, chlorambucil, chloroquine, cisplatin, cladribine, clodronate, cobimetinib, colchicine, crizotinib, cyclophosphamide, cyproterone, cytarabine, dabrafenib, dacarbazine, dactinomycin, daunorubicin, demethoxyviridin, dexamethasone, dichloroacetate, dienestrol, diethylstilbestrol, docetaxel, doxorubicin, EGFR inhibitors (e.g., tyrosine kinase inhibitors, Gefitinib, Osimertinib), epirubicin, eribulin, erlotinib, estradiol, estramustine, etoposide, everolimus, exemestane, filgrastim, fludarabine, fludrocortisone, fluorouracil and 5-fluorouracil, fluoxymesterone, flutamide, gefitinib, gemcitabine, genistein, goserelin, GSK1120212, hydroxyurea, idarubicin, ifosfamide, imatinib, interferon, irinotecan, ixabepilone, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, miltefosine, MK2206, mitomycin, mitotane, mitoxantrone, mutamycin, nilutamide, nocodazole, octreotide, olaparib, oxaliplatin, paclitaxel, pamidronate, pazopanib, pemetrexed, pentostatin, perifosine, PF-04691502, plicamycin, pomalidomide, porfimer, procarbazine, raltitrexed, ramucirumab, rituximab, romidepsin, rucaparib, selumetinib, sirolimus, sorafenib, streptozocin, sunitinib, suramin, talazoparib, tamoxifen, temozolomide, temsirolimus, teniposide, testosterone, thalidomide, thioguanine, thiotepa, titanocene dichloride, topotecan, trametinib, trastuzumab, tretinoin, vemurafenib, veliparib, vinblastine, vincristine, vindesine, vinorelbine, and vorinostat (SAHA). For example, chemotherapeutic agents that may be conjointly administered with therapeutic compounds include: aminoglutethimide, amsacrine, anastrozole, asparaginase, bcg, bicalutamide, bleomycin, bortezomib, buserelin, busulfan, campothecin, capecitabine, carboplatin, carfilzomib, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pentostatin, perifosine, plicamycin, pomalidomide, porfimer, procarbazine, raltitrexed, rituximab, sorafenib, streptozocin, sunitinib, suramin, tamoxifen, temozolomide, temsirolimus, teniposide, testosterone, thalidomide, thioguanine, thiotepa, titanocene dichloride, topotecan, trastuzumab, tretinoin, vinblastine, vincristine, vindesine, and vinorelbine. In other embodiments, chemotherapeutic agents that may be conjointly administered with therapeutic compounds include: ABT-263, dexamethasone, 5-fluorouracil, PF-04691502, romidepsin, and vorinostat (SAHA). In certain embodiments described herein, the chemotherapeutic agent conjointly administered with therapeutic compounds is a taxane chemotherapeutic agent, such as paclitaxel or docetaxel. In certain embodiments described herein, the chemotherapeutic agent conjointly administered with therapeutic compounds is doxorubicin. In certain embodiments described herein, a therapeutic compound is administered conjointly with a taxane chemotherapeutic agent (e.g., paclitaxel) and doxorubicin.

In some aspects, provided herein are an anti-cancer composition comprising an anti-cancer agent identified by the methods described herein. In some embodiments, the anti-cancer composition further comprises a proteasome inhibitor as described herein. In some embodiments, the proteasome inhibitor is bortezomib, carfilzomib, oprozomib, ixazomib, delanzomib, or an analog of any of these.

In some embodiments the method includes administering an immune checkpoint inhibitor, e.g., an antibody that binds to PD-1, PD-L1, CTLA-4, or another immune checkpoint protein.

Ins some embodiments, the copper ionophore enhances tumor cell death and/or immune cell death of the anti-cancer agent relative to the anti-cancer agent alone.

### Methods of Screening Anti-Cancer Agents

Some aspects of the disclosure are directed to a method of screening one or more test agents to identify a candidate anti-cancer agent, comprising contacting a cell sample (e.g., cancer cell) with a test agent, measuring a lipoylated protein (e.g., lipoyl-DLAT, lipoyl-DLST, lipoyl-GCSH, lipoyl-DBT) and identifying the test agent as a candidate anti-cancer agent if the level of the lipoylated protein is decreased as compared to a level of lipoylated protein of a corresponding cell sample not contacted with the test agent. The level of lipoylated protein of a corresponding cell sample not contacted with the test agent can be any suitable reference, such as a control sample or a reference sample, which in some embodiments may be representative of normal mitochondrial metabolism, and in other embodiments may be representative of increased mitochondrial metabolism. In some embodiments, the cell sample not contacted with the test agent does not express the lipoylated protein, or comprises a reduced level of the lipoylated protein.

In some embodiments of the invention, the test agent is identified as a candidate anti-cancer agent if a level of the lipoylated protein (e.g., lipoyl-DLAT, lipoyl-DLST, lipoyl-GCSH, lipoyl-DBT) is decreased by at least about 5%, 10%, 20%, 30%, 40%, 50%, 60%, 75%, 90%, 99% or more. In some embodiments of the invention, the test agent is identified as a candidate anti-cancer agent if a level of the lipoylated protein (e.g., lipoyl-DLAT, lipoyl-DLST, lipoyl-GCSH, lipoyl-DBT) is decreased by at least 1-fold, 2-fold, 3-fold, 5-fold, 6-fold, 7-fold, 8-fold, 9-fold, 10-fold or more.

In some embodiments, the method further comprises measuring a level or activity of a mitochondrial protein (e.g. FDX1, ALDHA1, ALDH2, LIAS, LIPT1, LIPT2, DLD) of the contacted cell sample and determining if the level or activity of the mitochondrial protein of the contacted cell is decreased as compared to a level or activity of the mitochondrial protein of a corresponding cell sample not contacted with the test agent.

In some embodiments of the invention, the test agent is identified as a candidate anti-cancer agent if a level or activity of the mitochondrial protein (e.g. FDX1, ALDHA1, ALDH2, LIAS, LIPT1, LIPT2, DLD, DLAT, DLST, DBT, GSH, Pyruvate dehydrogenase, 2-oxoglutarate dehydrogenase complex, Branched-Chain Alpha-Keto Acid Dehydrogenase Complex, glycine cleavage complex) is decreased by at least about 5%, 10%, 20%, 30%, 40%, 50%, 60%, 75%, 90%, 99% or more. In some embodiments of the invention, the test agent is identified as a candidate anti-cancer agent if a level or activity of the mitochondrial protein (e.g. FDX1, ALDHA1, ALDH2, LIAS, LIPT1, LIPT2, DLD, DLAT, DLST, DBT, GSH, Pyruvate dehydrogenase, 2-oxoglutarate dehydrogenase complex, Branched-Chain Alpha-Keto Acid Dehydrogenase Complex, glycine cleavage complex) is decreased by at least 1-fold, 2-fold, 3-fold, 5-fold, 6-fold, 7-fold, 8-fold, 9-fold, 10-fold or more.

In some embodiments, any assay capable of detecting expression of the relevant protein (e.g., lipoyl-DLAT, lipoyl-DLST, lipoyl-GCSH, lipoyl-DBT, FDX1, ALDHA1, ALDH2, LIAS, LIPT1, LIPT2, DLD, DLAT, DLST, DBT, GSH, Pyruvate dehydrogenase, 2-oxoglutarate dehydrogenase complex, Branched-Chain Alpha-Keto Acid Dehydrogenase Complex, glycine cleavage complex) can be used in the methods provided herein. In some embodiments, the proteins are detected by immunostaining with a labeled antibody that binds to the protein epitope. In some embodiments, the proteins are detected by immunohistochemistry. In some embodiments, the proteins are detected by Western Blot. In some embodiments, the mRNAs of the proteins are detected using qPCR. In some embodiments, the proteins are detected using fluorescence activated cell sorting (FACS). In some embodiments, the proteins are detected using microscopy (e.g., fluorescence microscopy). In some embodiments, the proteins are detected using ELISA.

In some embodiments, the method further comprises measuring cell death of the contacted cell sample and determining if cell death of the contacted cell is increased as compared to cell death of a corresponding cell sample not contacted with the test agent. The level of cell death of a corresponding cell sample not contacted with the test agent can be any suitable reference, such as a control sample or a reference sample, which in some embodiments may be representative of normal mitochondrial metabolism, and in other embodiments may be representative of increased mitochondrial metabolism.

For example, the reduction of Cu(II), bound to elesclomol by FDX1 to the toxic Cu(I) form promotes cell death in an increased mitochondrial metabolism state (as shown below).

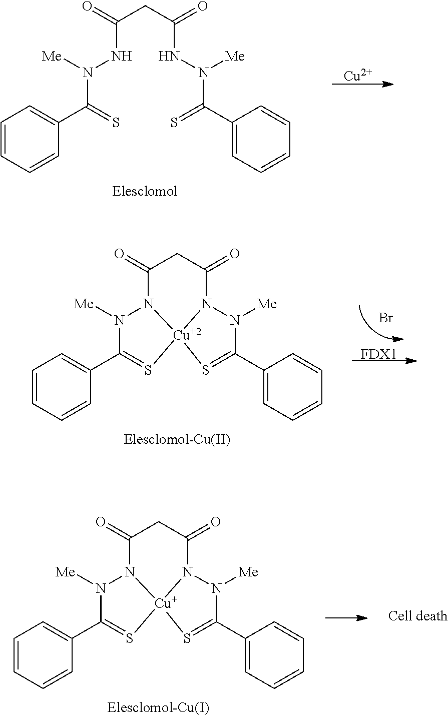

In some embodiments, any assay capable of detecting cell death after treatment with a test agent can be used in the methods provided herein. Cell death is typically characterized by membrane blebbing, condensation of cytoplasm, and the activation of endogenous endonucleases. Determination of any of these effects on cancer cells indicates that an Antibody-Drug Conjugate (ADC) is useful in the treatment of cancers.

Cell viability can be measured by determining in a cell the uptake of a dye such as neutral red, trypan blue, or ALAMAR™ blue (see, e.g., Page et al., 1993, Intl. J. Oncology 3:473-476). In such an assay, the cells are incubated in media containing the dye, the cells are washed, and the remaining dye, reflecting cellular uptake of the dye, is measured spectrophotometrically. The protein-binding dye sulforhodamine B (SRB) can also be used to measure cytoxicity (Skehan et al., 1990, J. Natl. Cancer Inst. 82:1107-12).

Alternatively, a tetrazolium salt, such as MTT, is used in a quantitative colorimetric assay for mammalian cell survival and proliferation by detecting living, but not dead, cells (see, e.g., Mosmann, 1983, J. Immunol. Methods 65:55-63).

Cell death can be quantitated by measuring, for example, DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica, 1999, no. 2, pp. 34-37 (Roche Molecular Biochemicals).

Cell death can also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). A method for measuring cell death number has been described by Duke and Cohen, Current Protocols in Immunology (Coligan et al. eds., 1992, pp. 3.17.1-3.17.16). Cells also can be labeled with a DNA dye (e.g., acridine orange, ethidium bromide, or propidium iodide) and the cells observed for chromatin condensation and margination along the inner nuclear membrane. Other morphological changes that can be measured to determine cell death include, e.g., cytoplasmic condensation, increased membrane blebbing, and cellular shrinkage.

The presence of cell death can be measured in both the attached and “floating” compartments of the cultures. For example, both compartments can be collected by removing the supernatant, trypsinizing the attached cells, combining the preparations following a centrifugation wash step (e.g., 10 minutes at 2000 rpm), and detecting cell death (e.g., by measuring DNA fragmentation). (See, e.g., Piazza et al., 1995, Cancer Research 55:3110-16).

In certain aspects, provided herein is a method of determining increased mitochondrial metabolism in a tumor and/or immune cell, comprising staining for lipoic acid in the tumor and/or immune cell.

In certain aspects, provided herein is a method of identifying a candidate anti-cancer agent, comprising the steps of (a) incubating a cell sample with copper-supplemented media; (b) contacting a cell sample with a test agent; (c) measuring cell viability of the cell sample; and (d) identifying the test agent as a candidate anti-cancer agent if the level of cell viability is decreased as compared to a level of cell viability of a cell sample incubated with copper-supplemented media and not contacted with the test agent. The level of cell viability of a corresponding cell sample not contacted with the test agent can be any suitable reference, such as a control sample or a reference sample, which in some embodiments may be representative of normal mitochondrial metabolism, and in other embodiments may be representative of increased mitochondrial metabolism.

In certain aspects, provided herein is a method of identifying a candidate anti-cancer agent, comprising the steps of (a) incubating a cell sample with a copper chelator; (b) contacting a cell sample with a test agent; (c) measuring cell death of the cell sample; and (d) identifying the test agent as a candidate anti-cancer agent if the level of cell death is decreased as compared to a level of cell death of a cell sample incubated with a copper chelator and not contacted with the test agent. The level of cell death of a corresponding cell sample not contacted with the test agent can be any suitable reference, such as a control sample or a reference sample, which in some embodiments may be representative of normal mitochondrial metabolism, and in other embodiments may be representative of increased mitochondrial metabolism.

In some embodiments, the copper chelator is tetrathiomolybdate (TTM). An example of chelation of copper by TTM is shown below.

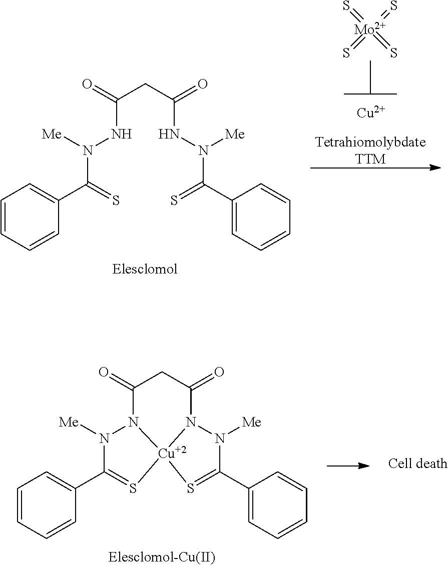

In certain aspects, provided herein is a method of identifying a candidate anti-cancer agent, comprising the steps of (a) incubating a cell sample with metal-supplemented media; (b) contacting a cell sample with a test agent; (c) measuring cell viability of the cell sample; and (d) identifying the test agent as a candidate anti-cancer agent if the level of cell viability is decreased as compared to a level of cell viability of a cell sample incubated with metal-supplemented media or and not contacted with the test agent.

In one embodiment, the metal-supplemented media is zinc-supplemented media (Zn-supplemented media). In another embodiment, the metal-supplemented media is manganese-supplemented media (Mn-supplemented media). In another embodiment, the metal-supplemented media is cobalt-supplemented media (Co-supplemented media). In yet another embodiment, the metal-supplemented media is nickel-supplemented media (Ni-supplemented media). In yet another embodiment, the metal-supplemented media is iron-supplemented media (Fe-supplemented media).

In certain aspects, provided herein is a method of identifying a candidate anti-cancer agent, comprising the steps of (a) incubating a cell sample with a metal chelator; (b) contacting a cell sample with a test agent; (c) measuring cell death of the cell sample; and (d) identifying the test agent as a candidate anti-cancer agent if the level of cell death is decreased as compared to a level of cell death of a cell sample incubated with a metal chelator and not contacted with the test agent.

In one embodiment, the metal chelator is a zinc (Zn) chelator. In another embodiment, the metal chelator is a manganese (Mn) chelator. In another embodiment, the metal chelator is a cobalt (Co) chelator. In yet another embodiment, the metal chelator is a nickel (Ni) chelator. In yet another embodiment, the metal chelator is an iron (Fe) chelator.

In certain aspects, provided herein is a method of identifying a candidate anti-cancer agent, comprising the steps of (a) incubating a cell sample with glucose-supplemented media; (b) removing the glucose-supplemented media and then incubating the cell sample with galactose-supplemented media; (c) contacting the cell sample with a test agent; (d) measuring cell viability of the cell sample; and (e) identifying the test agent as a candidate anti-cancer agent if the level of cell viability is decreased as compared to a level of cell viability of a cell sample first incubated with glucose-supplemented media, then incubated with galactose-supplemented media after removing the glucose-supplemented media, and not contacted with the test agent. The level of cell death of a corresponding cell sample not contacted with the test agent can be any suitable reference, such as a control sample or a reference sample, which in some embodiments may be representative of normal mitochondrial metabolism, and in other embodiments may be representative of increased mitochondrial metabolism.

In certain aspects, provided herein is a method of identifying a candidate anti-cancer agent, comprising the steps of (a) incubating a cell sample in media, wherein the cell sample comprises a deletion in a gene encoding a mitochondrial protein; (b) contacting a cell sample with a test agent; (c) measuring cell viability of the cell sample; and (d) identifying the test agent as a candidate anti-cancer agent if the level of cell viability is increased as compared to a level of cell viability of a cell sample not comprising a deletion in the gene encoding the mitochondrial protein and contacted with the test agent. In some embodiments, the mitochondrial protein is ferredoxin 1 (FDX1). The level of cell viability of a corresponding cell sample comprising a deletion in the gene encoding the mitochondrial protein and contacted with the test agent can be any suitable reference, such as a control sample or a reference sample, which in some embodiments may be representative of normal mitochondrial metabolism, and in other embodiments may be representative of increased mitochondrial metabolism.

In certain aspects, provided herein is a kit for identifying a candidate anti-cancer agent comprising a test agent, and an assay for measuring cellular protein lipoylation.

In certain aspects, provided herein is a kit for identifying a candidate anti-cancer agent comprising copper-supplemented media, a test agent, and an assay for measuring cell viability.

In certain aspects, provided herein is a kit for identifying a candidate anti-cancer agent comprising a copper chelator, a test agent, and an assay for measuring cell death.

### EXEMPLIFICATION

Elesclomol is a compound developed as an anti-cancer therapeutic. Synta Pharmaceuticals generated results in a Phase II trial that suggested that elesclomol may provide a cancer progression-free survival benefit in combination with paclitaxel (O'Day et al., J Clin Oncol, 2009, incorporated by reference in its entirety). However, the subsequent Synta Pharmaceuticals Phase III trial failed. Post-hoc analysis of Phase III trial results revealed a correlation between a high level lactate dehydrogenase (LDH) and elesclomol efficacy (O'Day et al., J Clin Oncol, 2013, incorporated by reference in its entirety). LDH is a key enzyme in anaerobic respiration. Potential reasons for the failure of the Phase III trial were that 1) the mechanism of action of elesclomol was unknown, 2) no biomarker was available for patient selection, 3) elesclomol was administered in a sub-optimal formulation.

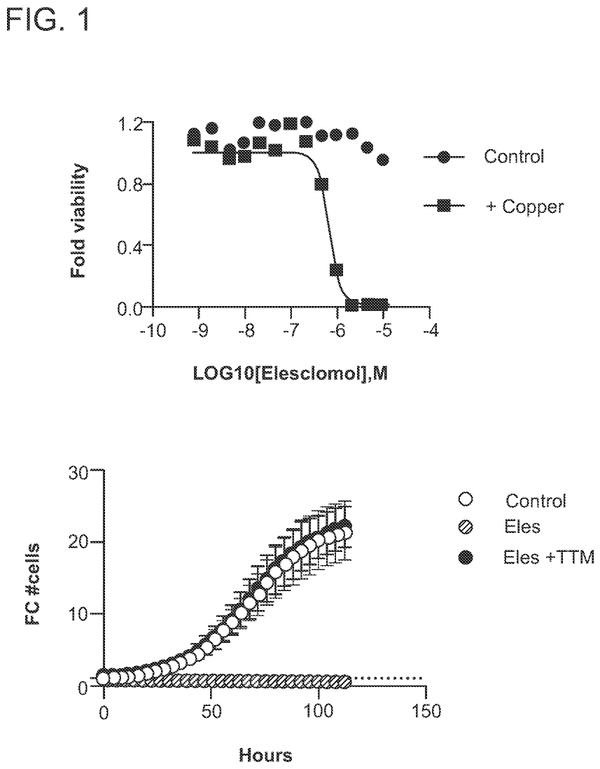

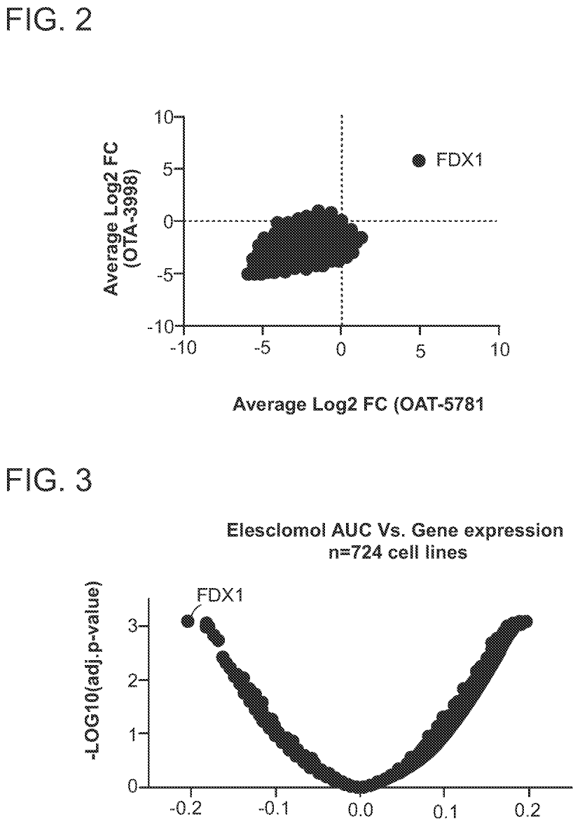

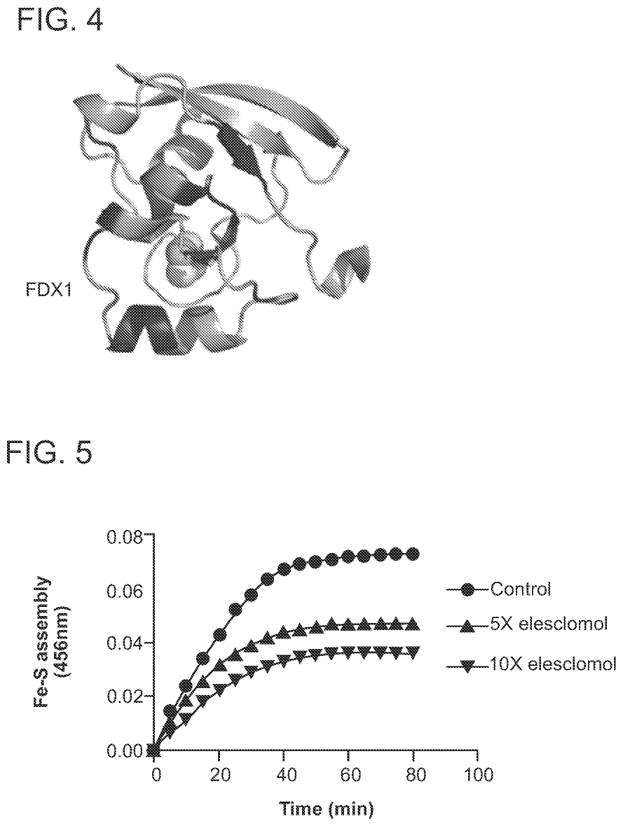

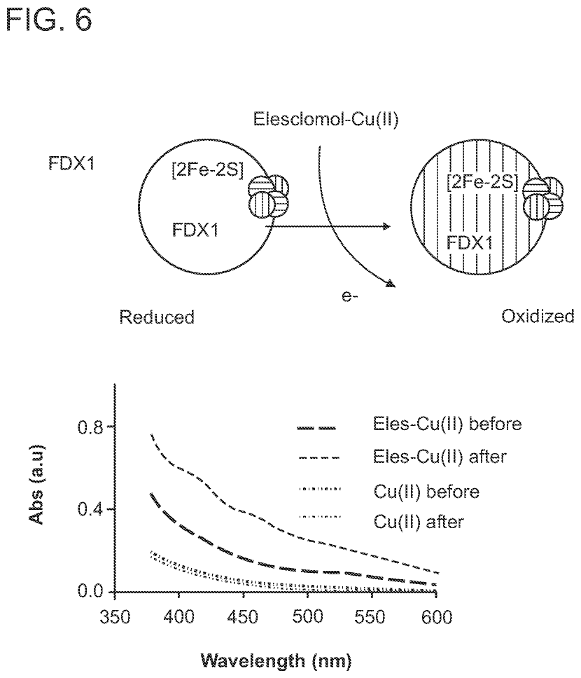

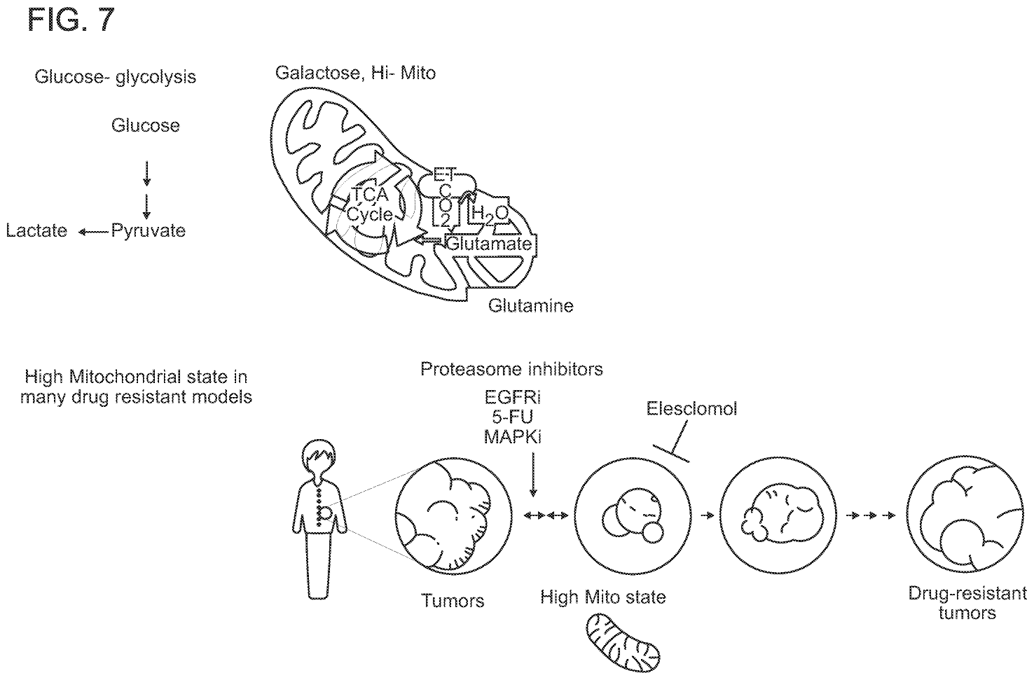

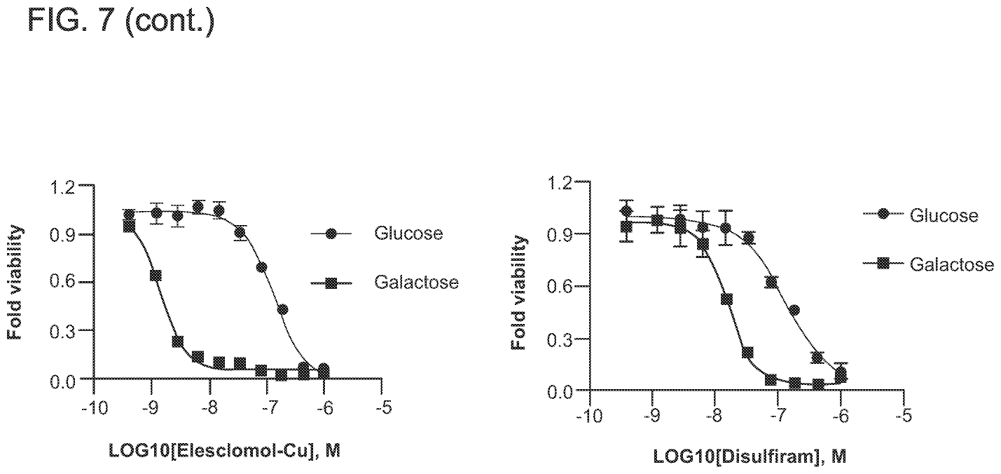

Recent findings reported in Tsvetkov et al., Nat Chem Bio, 2019 reveal that elesclomol killing is copper dependent (FIG. 1). Furthermore, mitochondrial protein ferredoxin 1 (FDX1) was found to be the direct target of elesclomol (FIG. 4). Whole genome CRISPR rescue screens revealed deletion of FDX1 confers resistance to two distinct elesclomol analogs (FIG. 2). Additionally, PRISM biomarker analysis revealed high expression of FDX1 correlates with increased sensitivity to elesclomol (FIG. 3). Tsvetkov et al., Nat Chem Bio, 2019 demonstrated elesclomol inhibits FDX1 activity in vitro and elesclomol-Cu(II) is a neo-substrate of FDX1 (FIG. 5 & FIG. 6). The authors demonstrated that elevated levels of mitochondrial metabolism, which is present in many drug resistant models, predicts elesclomol sensitivity (FIG. 7).

### Example 1: FDX1 Regulates the Lipoic Acid Pathway

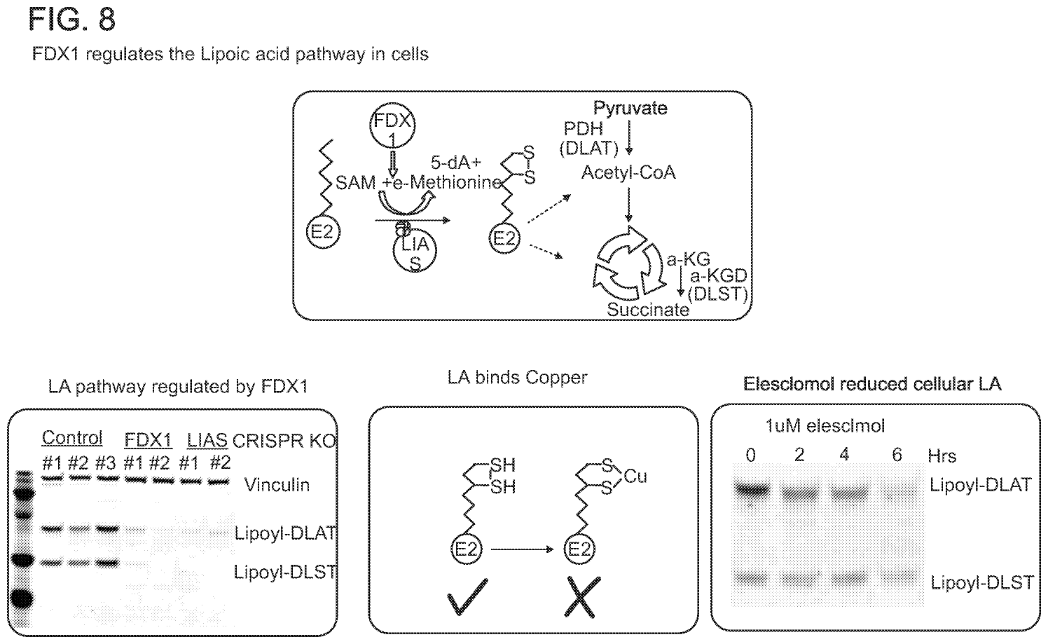

CRISPR KO FDX1 cell lines or CRISPR KO LIAS cell lines were created and were assessed for levels of lipoylated proteins lipoyl-DLAT and lipoyl-DLST by Western Blot (FIG. 8). Western blot analysis revealed lipoylated protein levels decreased dramatically in CRISPR KO cells lines compared to control cells. Furthermore, treatment of cells with 1 μM elesclomol over 6 hours showed that lipoyl-DLAT and lipoyl-DLST protein levels decreased over time. In conclusion, FDX1 was found to regulate the lipoic acid pathway (FIG. 8).

### Example 2: Lipoic Acid Stain is a Biomarker for Sensitivity to Elesclomol

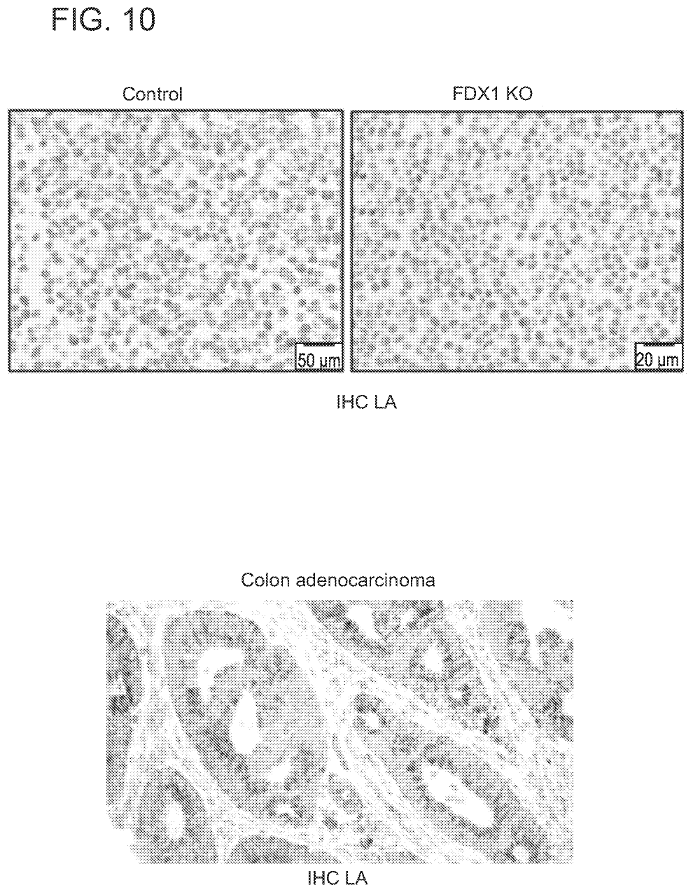

Lipoic acid was stained in FDX1 KO cells by an immunohistochemistry assay. Microscopy images demonstrated that lipoic acid levels were dramatically decreased in FDX1 KO cells compared to the control cells (FIG. 10). Staining of colon adenocarcinoma tissue demonstrated that lipoic acid levels were elevated in the tissue. In conclusion, staining for lipoic acid in tumors can serve as a biomarker for tumors with elevated levels of mitochondrial metabolism. Additionally, increased levels of lipoic acid in tumors were found to be sensitive to copper-bound compounds such as elesclomol and disulfiram.

### Example 3: Different Compounds Promote Copper Dependent Cell Death

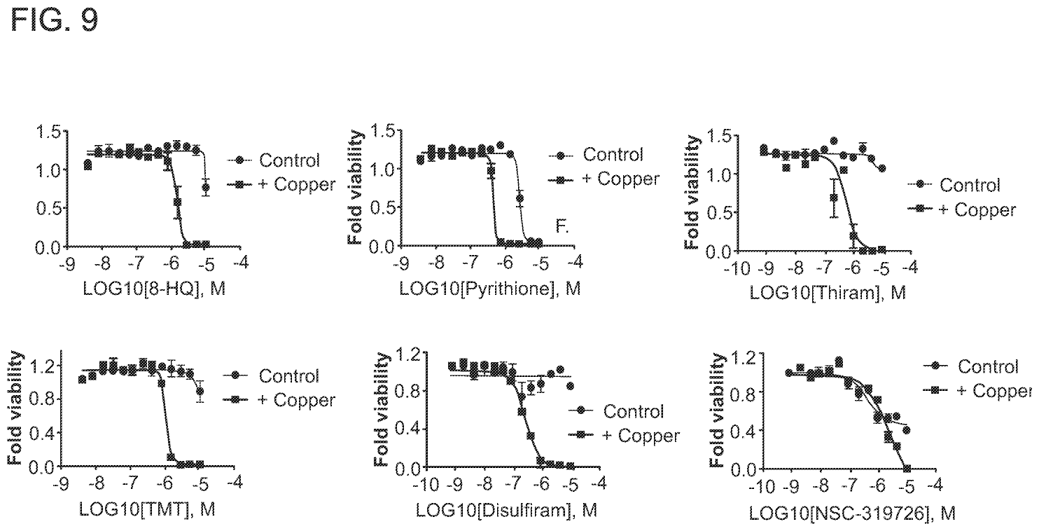

In addition to elesclomol, other compounds were found to promote copper dependent cell death in cancer cells (FIG. 9).

### Example 4: Clustering of Compound Viability Profiles Reveals a Unique Cluster of Compounds that Promote Copper Dependent Cell Death

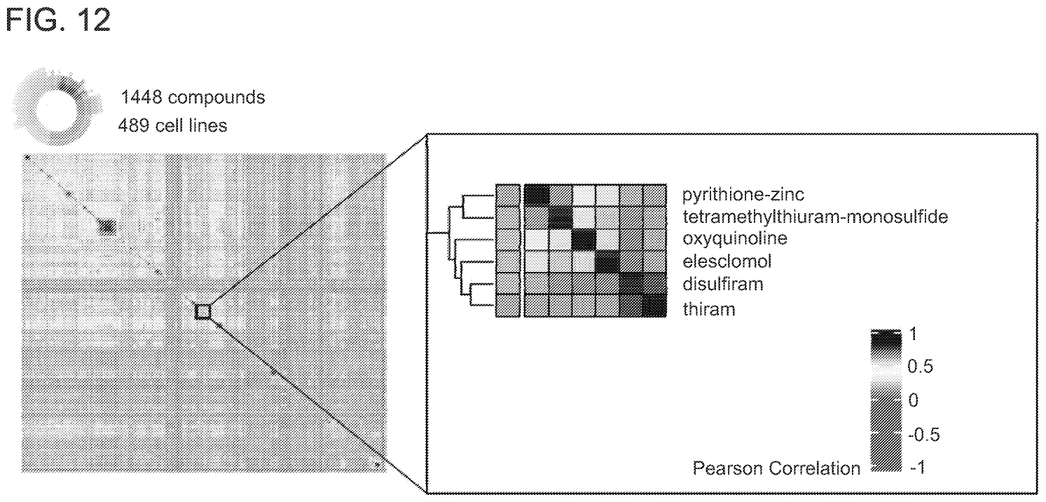

To reveal new unique pathway targeting drugs that can promote cancer cell death, the clustering of compound viability profiles testing 1448 compounds on 489 cell lines was analyzed. This analysis revealed a unique cluster of metal binding molecules (FIG. 12). Interestingly many of these compounds were shown to specifically bind copper. The anti-abuse compound disulfiram and structurally similar analogs, such as Thiram and tetra methyl thiuram monosulfide (TMT), were shown to promote copper dependent phenotypes. Oxyquinoline (8-HQ) that binds different metals, can promote a copper dependent induction of a-beta proteasome mediated degradation and pyrithione that promotes copper-dependent cell death in yeast. In particular, the compound elesclomol, which binds and shuttles copper to the mitochondria and exerts both beneficial and detrimental outcomes on the cell, was of interest. The clustering of these copper binding molecules in a unique module suggests that certain cells might have a unique sensitivity to copper binding molecules, which is distinct from other explored compounds in this cohort.

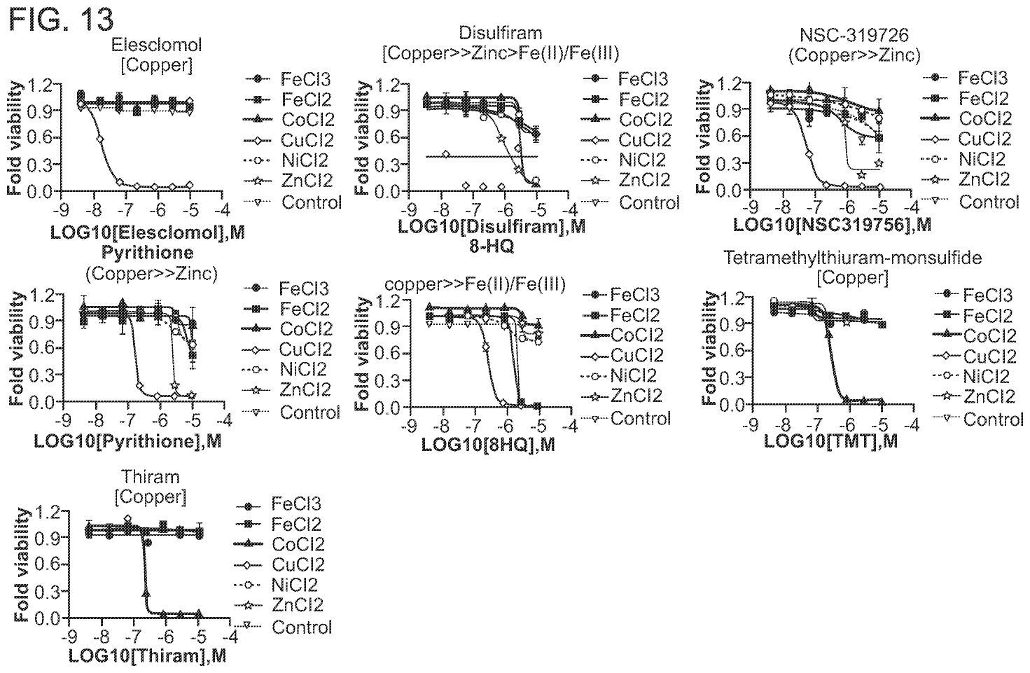

Metal binding compounds can induce a toxic phenotype either by sequestration (chelation) of metals from essential factors or by shuttling (e.g., an ionophore) of metals to cellular compartments where they are toxic. To determine if the compounds observed in the cluster promote cell death by chelation or the shuttling of certain metals, the cell toxicity induced by each compound in the presence or absence of Iron, Cobalt, Copper, Nickel and Zinc was examined. In all examined cases, the addition of copper strongly augmented the cell death induction by all compounds in the cluster (FIG. 13). To a lower extent, Zinc with Disulfiram, NSC319756, or Pyrithione and Iron with 8HQ were also able to promote the cell death. Thus, copper binding and copper-induced cell death is a common phenotype of the distinct cluster of compounds.

### Example 5: Copper Induced Cell Death is Non-Apoptotic or Ferroptotic

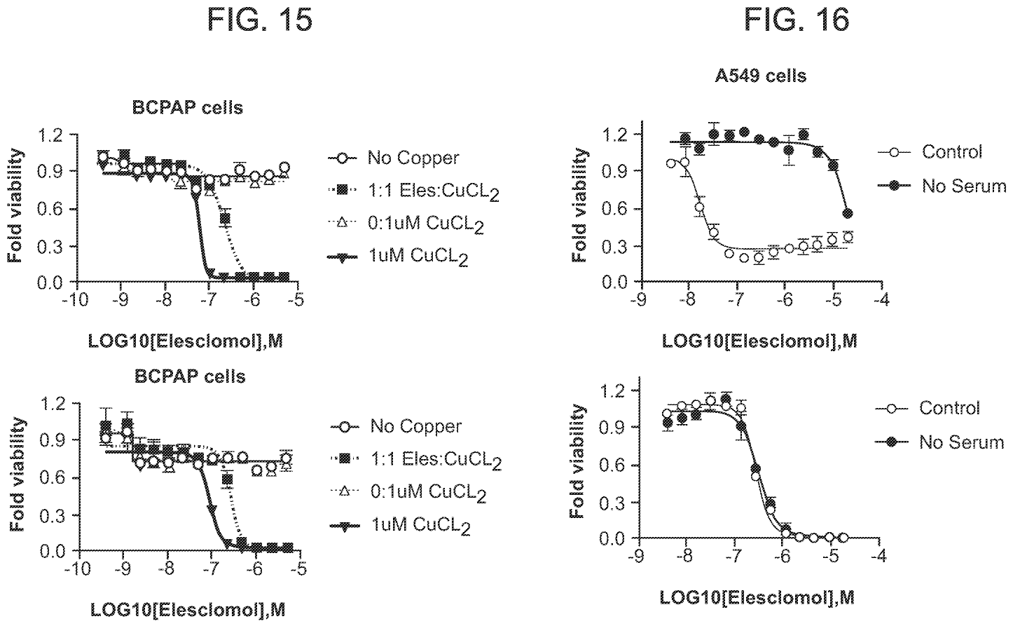

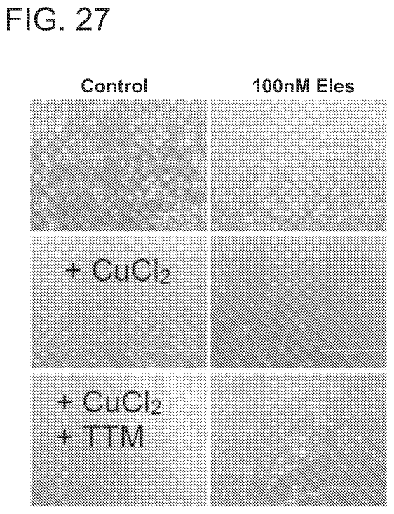

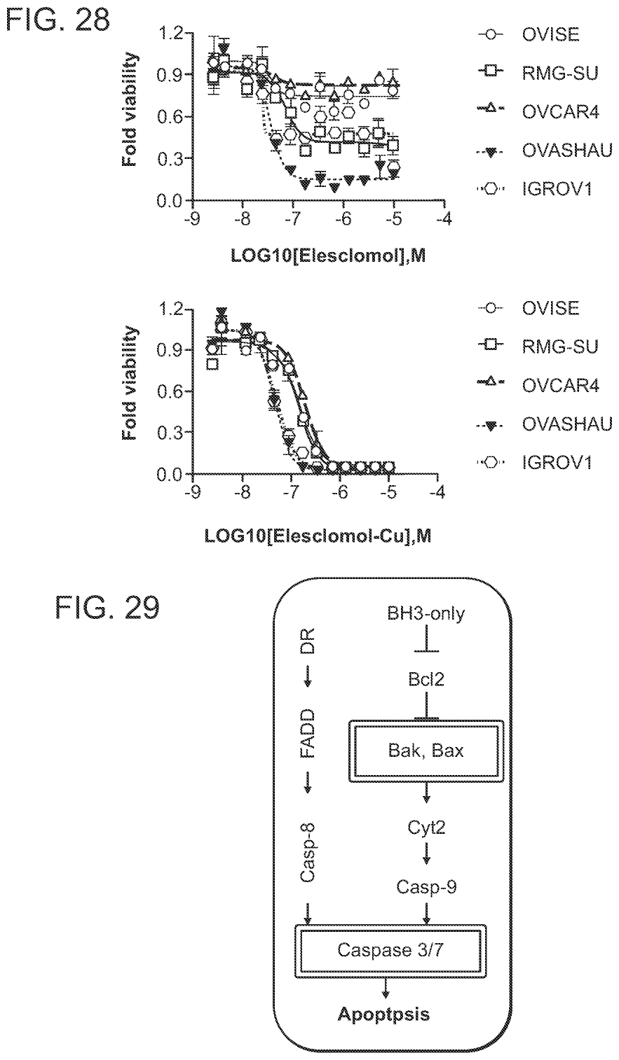

Elesclomol shows the most selective binding to copper in the cluster (FIG. 13). The sensitivity of cells to elesclomol is dependent on copper availability. The addition of copper either in the media at physiological concentrations (1 μM) or at 1:1 ratio with the compound, both strongly augment elesclomol-induced cell death (FIG. 15). Chelation of copper from the media with tetrathiomolybdate (TTM) completely blocks the elesclomol induced cell death in multiple cell line models (FIG. 27). Copper is not supplemented in the media (RPMI) and therefore the availability of copper is restricted to the abundance of copper in serum, which can strongly vary. As such, cells treated with elesclomol in media lacking serum show increased resistance that is completely reversed by the addition of copper to the media (FIG. 16). The supplementation of copper to the media strongly reduces the variability of elesclomol efficacy as shown for a subset of ovarian cancer cell lines (FIG. 28), suggesting that intracellular copper levels may also dictate the sensitivity to elesclomol when exogenous copper levels are low. Thus, the efficacy of elesclomol is dependent on extracellular and intracellular copper availability.

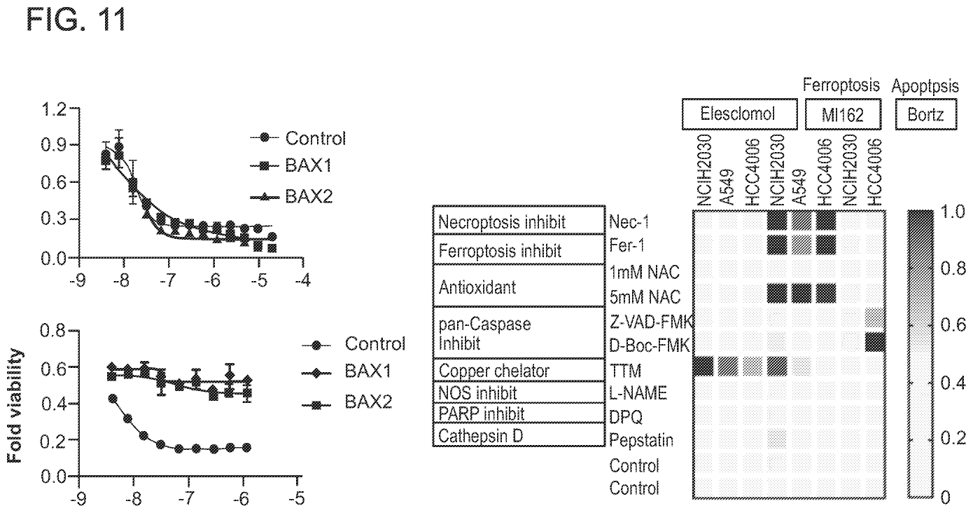

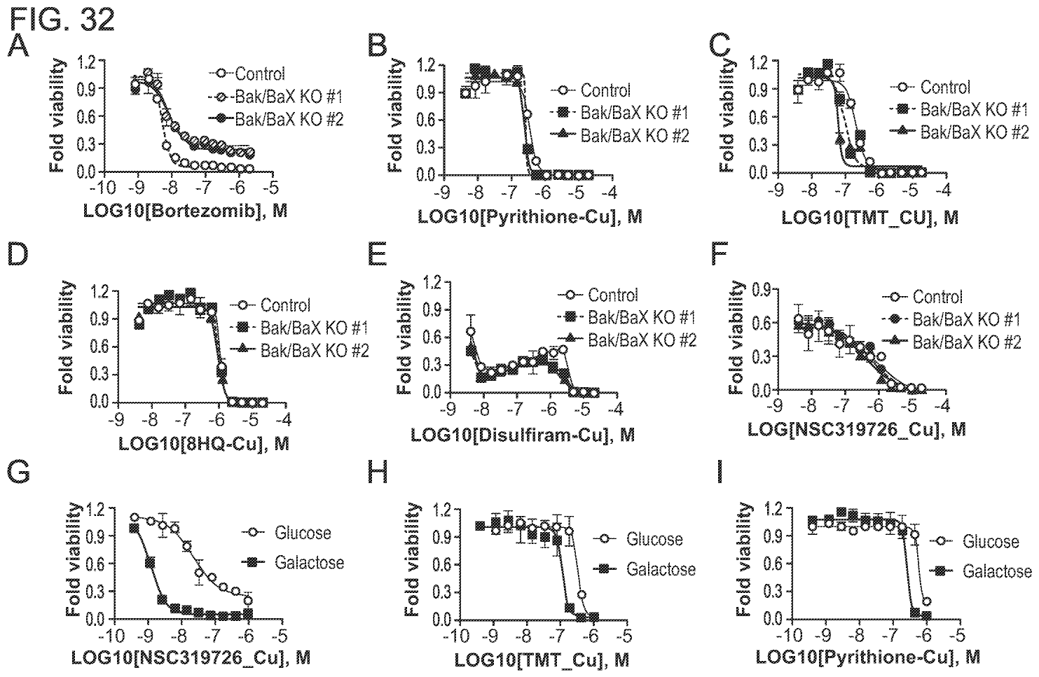

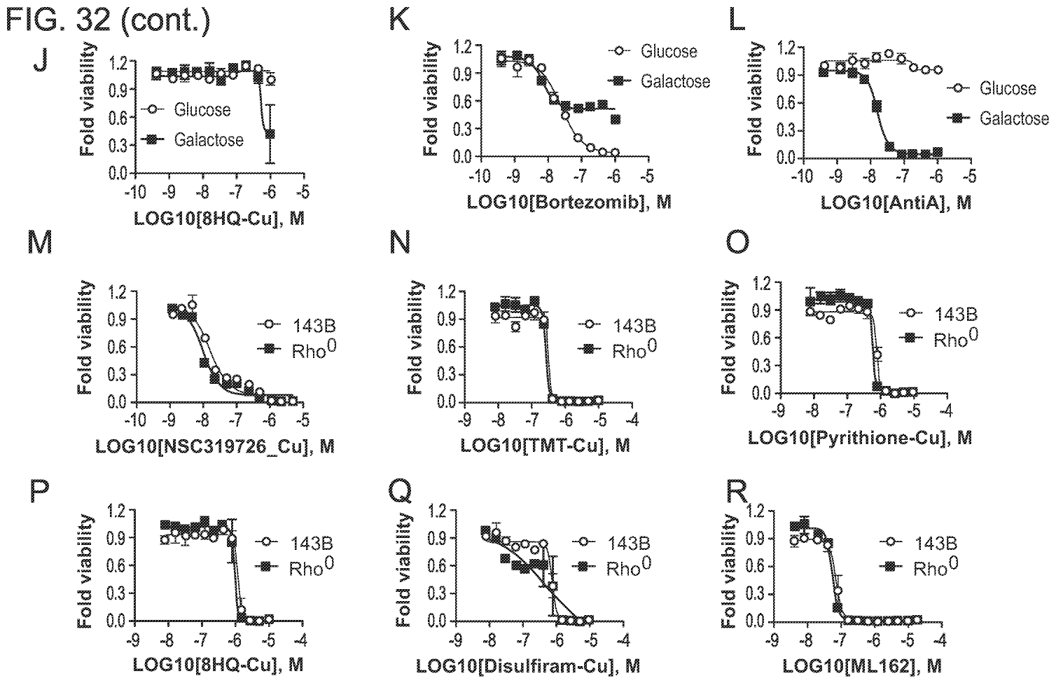

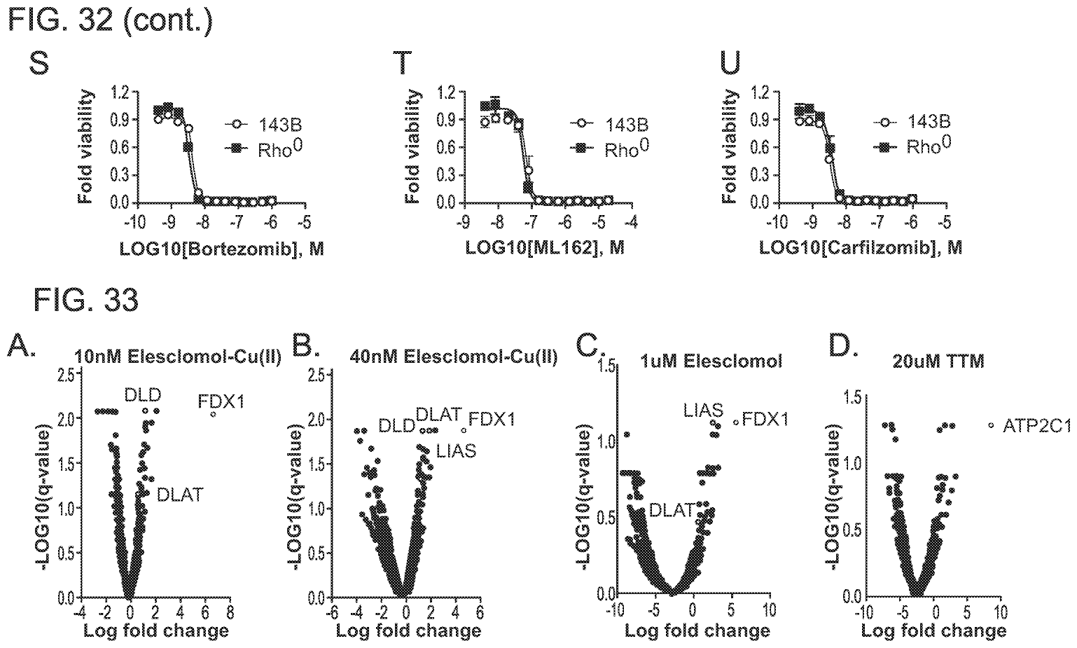

Both iron and copper can generate highly reactive hydroxyl radical (.OH) from O₂.— and H₂O₂ in the cell via the Fenton reaction. In the case of iron, this can result in lipid radicals that induce ferroptosis, a non-apoptotic cell death. Previous findings suggested that elesclomol induced a ROS-dependent apoptotic cell death. However, it was recently shown that elesclomol-induced cell death does not involve significant caspase-3 activation. As such, to determine if elesclomol induced cell death is apoptotic, a genetic approach was undertaken using the HCM18 cells that have the crucial apoptosis effectors (Bak and Bax) knocked out (FIG. 29). In the Bak/Bax deleted cells the efficacy of apoptosis-inducing paclitaxel strongly inhibited, as expected (FIG. 11). However, the ability of elesclomol-copper to induce cell death was unaffected (FIG. 11). Other copper binding molecules were tested in these cell lines and in all cases the efficacy of these compounds was unaffected by the genetic perturbation of the apoptosis pathway (FIG. 32).

To further establish which cell death pathways might be mediating the elesclomol induced copper-dependent cell death, a chemical approach was undertaken using compounds that block critical niches of known cell death pathways. Pre-treatment of cells with compounds that block apoptosis (pan-caspase inhibitors), Ferroptosis (Ferrostatin-1), necroptosis (Necrostatin-1) and other antioxidants and cell death regulating pathways did not block the elesclomol-copper induced cell death. As controls, ferroptosis-inducing GPX4 inhibitor (ML162) and apoptosis-inducing proteasome inhibitor (bortezomib) were used. In the three cell lines tested only copper chelation by TTM, was able to block elesclomol induced cell death (FIG. 11). Ferroptosis or apoptosis-altering compounds (as shown in the ML162 and bortezomib controls) had no effect on elesclomol-copper-induced cell death. Consistent with these findings, elesclomol-copper did not induce a profound change in lipid peroxidation that is commonly observed during ferroptosis-induction. The slight changes observed are most likely due to the copper chelating properties of the molecule. Elesclomol-copper showed increased toxicity and decreased levels of lipid peroxidation and copper chelation by TTM. All together, these findings suggest that elesclomol-copper, and other copper binding compounds induce cell death that is chemically and genetically distinct from both apoptosis and ferroptosis. Given the absolute dependency and regulation of this cell death pathway by copper, this process is referred to as ‘cuproptosis’ for simplicity.

### Example 6: FDX1 Regulated Lipoylation is a Crucial Regulator of Sensitivity to Elesclomol

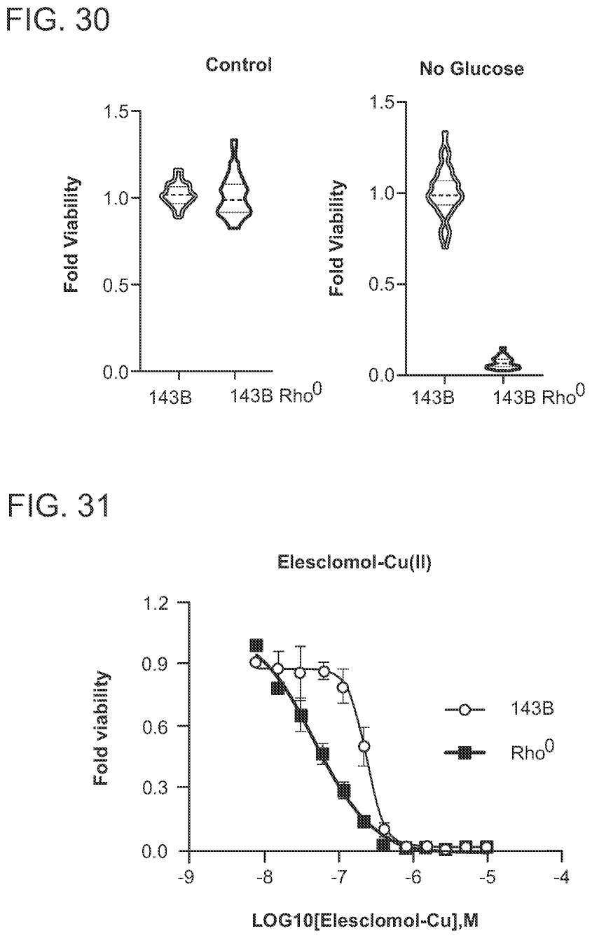

Elesclomol-induced cuproptosis is regulated by mitochondrial metabolism. Cells that are forced to switch to increased mitochondrial metabolism by replacement of glucose with galactose in the media become increasingly more sensitive to elesclomol (FIG. 7 and FIG. 30). This shift to mitochondrial metabolism also strongly potentiates the efficacy of the other copper-binding molecules, some exhibiting stronger effects than others. Whereas the effect of mitochondrial metabolism on 8-HQ, Pyrithione, and TMT are rather mild, the effect for disulfiram and NSC-319726 were similar to those shown for elesclomol (FIG. 32). To establish if this effect is mediated by the electron transfer chain (ETC), 143B rho0 cells (lacking mitochondrial DNA) and their parental controls (143B) were tested. Surprisingly, the 143B rho0 cells were slightly more sensitive cell death, in the case of elesclomol and disulfiram, or equally sensitive cell death, in the case of TMT, NSC319726, Pyrithione and 8HQ, by the copper binding compounds (FIG. 31, FIG. 32). This ruled out the possibility that elesclomol, and other copper binding compounds require a functional ETC to promote cell death.

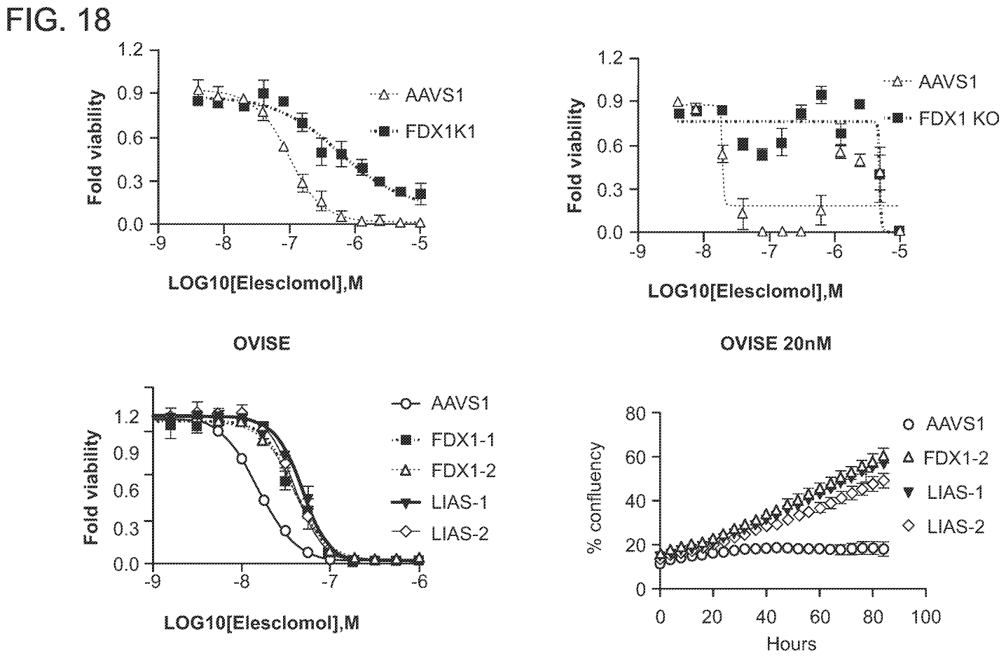

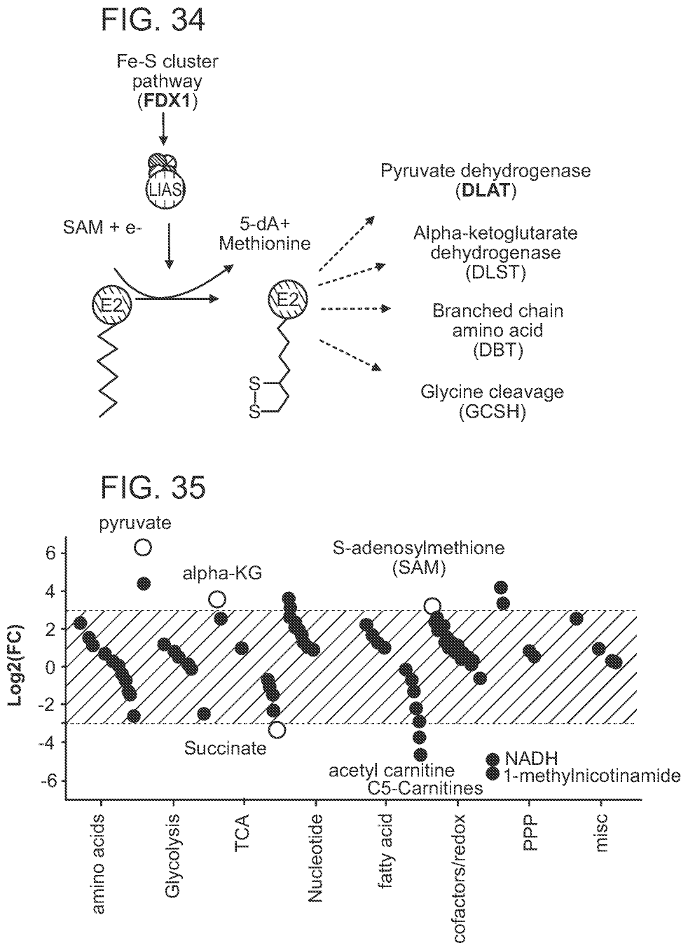

It has been previously shown that ferredoxin 1 (FDX1) is an important mediator of elesclomol-induced toxicity. Elesclomol directly binds to FDX1, which can reduce elesclomol-bound copper to promote cuproptosis. To better understand the potential downstream mediators of cuproptosis, a CRISPR/Cas9 deletion strategy was used. A targeted screen focused on 3000 metabolic enzymes to identify genes that lose conferred resistance to both elesclomol alone and when combined with copper supplementation in the adherent lung cancer cell line A549. In all conditions, FDX1 gene was the highest scoring hit, emphasizing the importance of this gene in elesclomol mediated toxicity (FIG. 33). Interestingly, there were multiple hits from two distinct functional pathways: mitochondrial complex I and the lipoic acid pathway. The lipoic acid pathway mediates the lysine lipoylation of specific enzymes (DLAT, DLST, DBT, GCSH) that is crucial for their function (FIG. 34). Interestingly, both the lipoylating enzymes (LIAS) and the downstream lipoylated target enzymes (PDH complex) were hits in the genetic modifier screen. This strongly suggested that the lipoylated state of the cell plays a role in mediating elesclomol-induced toxicity. This hypothesis was particularly appealing as lipoylation is required for mitochondrial respiration and lipoic acid has shown to have a high affinity binding to copper. Findings were validated and showed that indeed deletion of FDX1 conferred resistance to elesclomol-Cu(II) and also to disulfiram-Cu(II) in multiple cell models (FIG. 18). Deletion of the lipoylation enzyme LIAS was also sufficient to promote resistance to elesclomol (FIG. 18).

### Example 7: FDX1 is an Upstream Regulator of Lipoylation

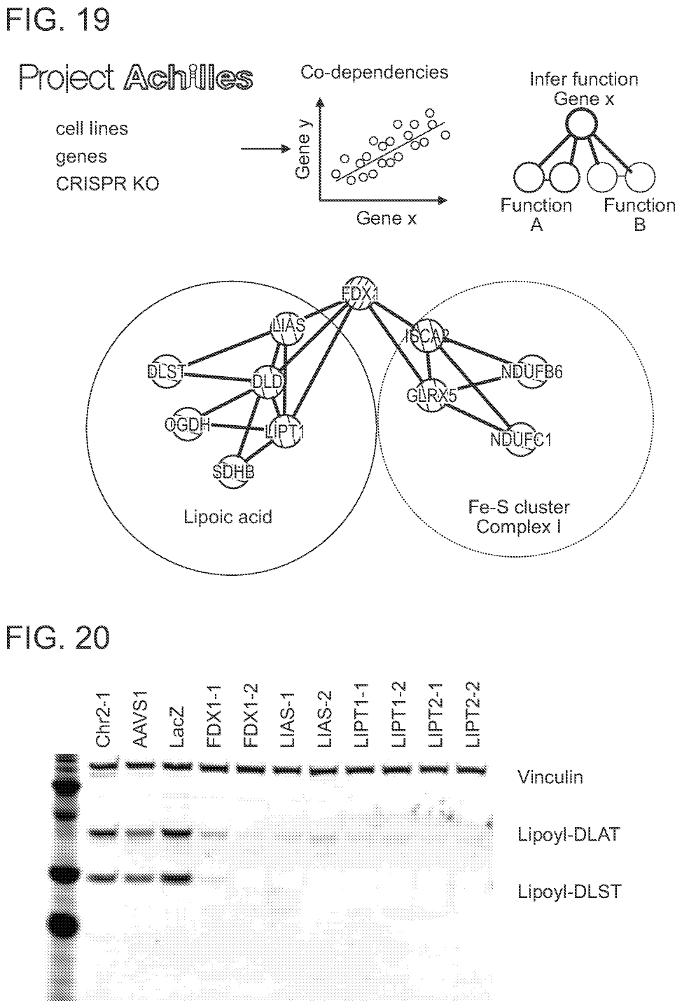

Despite numerous studies focused on FDX1, the natural function of FDX1 in the cell is still debated. On one hand, FDX1 was shown to participate in the mitochondrial Fe—S cluster pathway. And on the other hand, there is evidence that contradicts these findings suggesting FDX1 has a different role. To determine the role of FDX1 in cancer cells, an analysis was performed to identify which genes show a similar viability effect when knocked out by CRISPR/Cas9 across hundreds of cancer cell lines. This analysis revealed that FDX1 deletion highly correlates with proteins from two functional categories, the mitochondrial complex I and the Lipoic acid pathway (FIG. 19). These results are almost identical to the functional hits in the genetic deletion rescue screen using elesclomol (FIG. 33). These results strongly suggesting that both FDX1 and Lipoic acid pathway genes are in the same functional pathway.

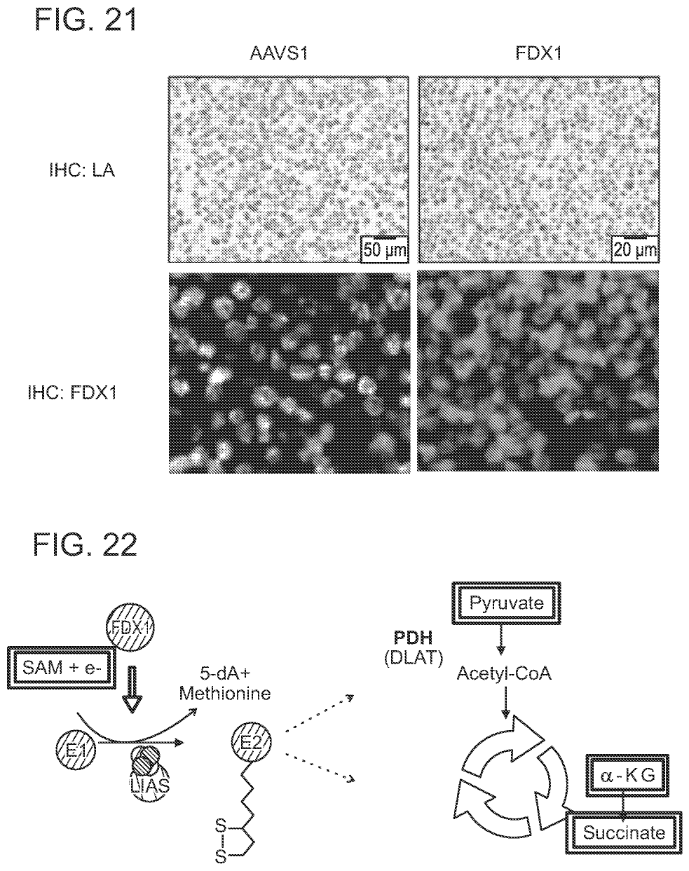

To establish if FDX1 is upstream of protein lipoylation pathway, FDX1 and other critical enzymes in the lipoylation pathway (LIPT1, LIAS, LIPT2) were knocked out. The lipoylated state of DLAT and DLST was assessed by using an antibody which recognizes lipoylated lysine. FDX1 deletion, like that of the established LA pathway enzymes, completely abolished the levels of lipoylated DLAT and DLST as observed by both Western blot analysis and IHC (FIG. 20 and FIG. 21).

From the assessment, many metabolites were altered in the FDX1 KO cells compared to both the AAVS1-targeting control and K562 parental cells, which is consistent with the newly discovered role of FDX1 in regulation of protein lipoylation (FIG. 35). In particular, there is accumulation of pyruvate and a-KG with depletion of succinate, which is expected when the activity of DLAT and DLST is inhibited (FIG. 22). There was also a strong increase in the NAD/NADH ratio with no significant changes in lactate levels. Interestingly, there is also accumulation of overall SAM levels, suggesting that the SAM consumed by protein lipoylation in these cells constitutes a significant portion of overall cellular SAM. Taken together, the metabolomics data strongly supports the findings that FDX1 is an upstream regulator of protein lipoylation.

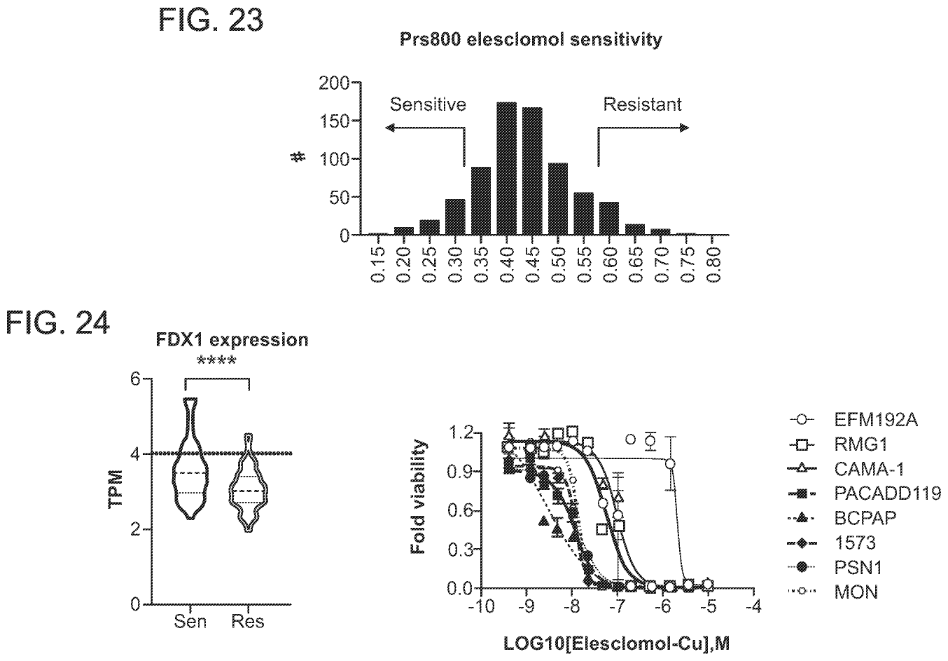

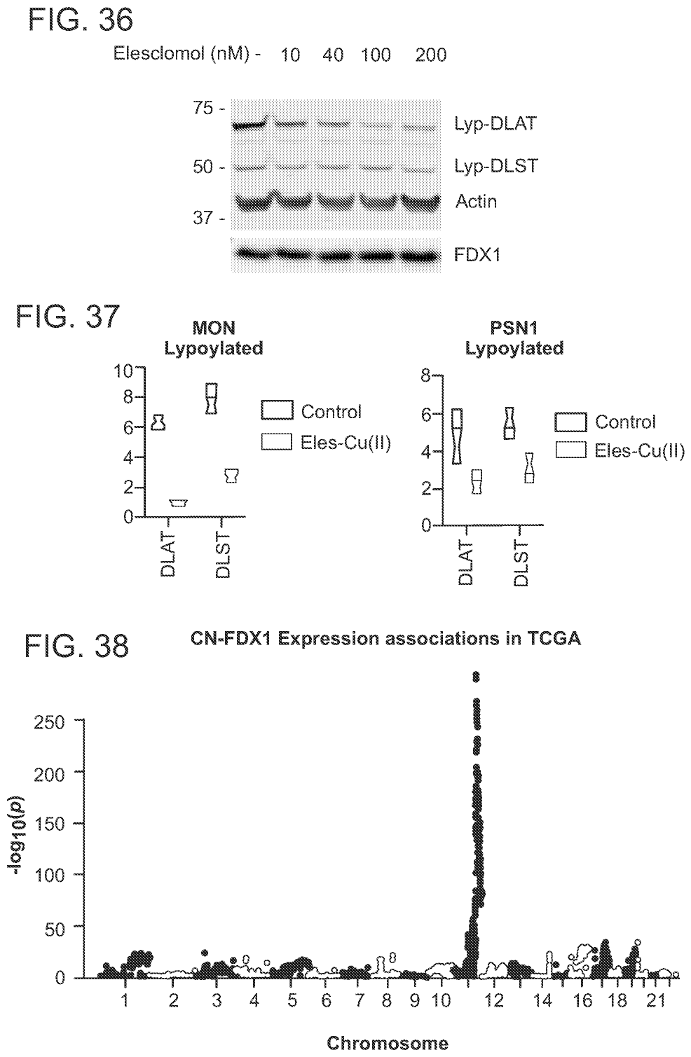

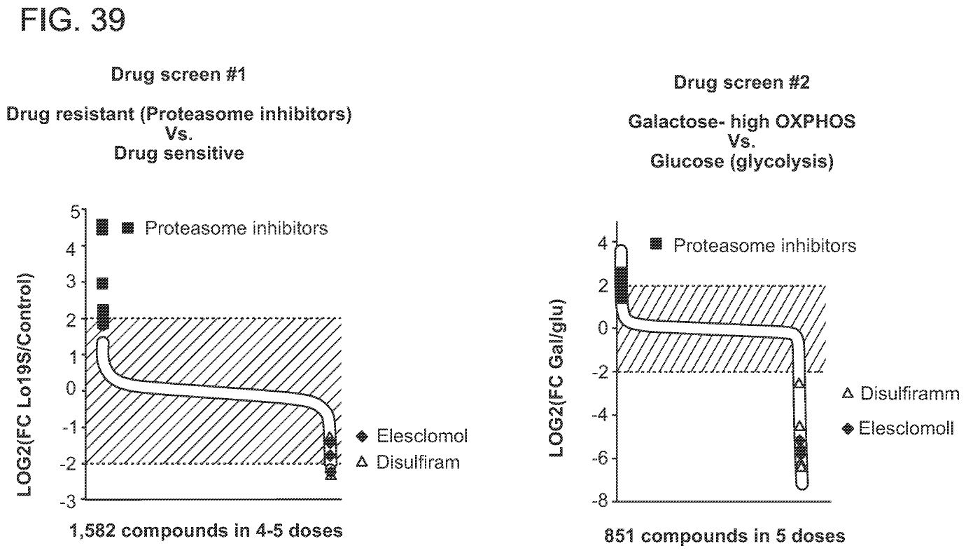

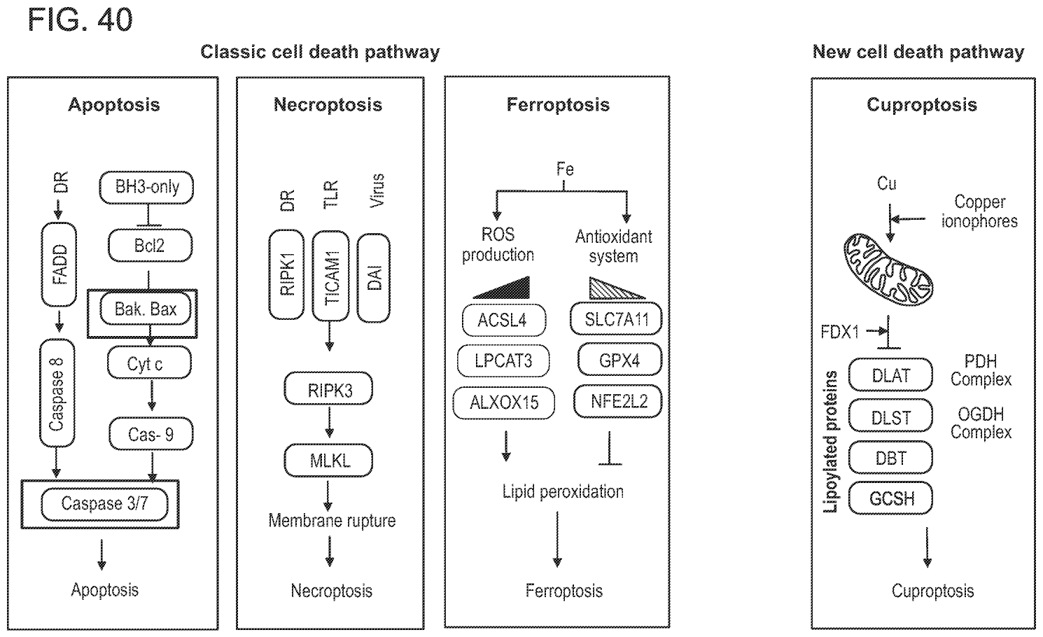

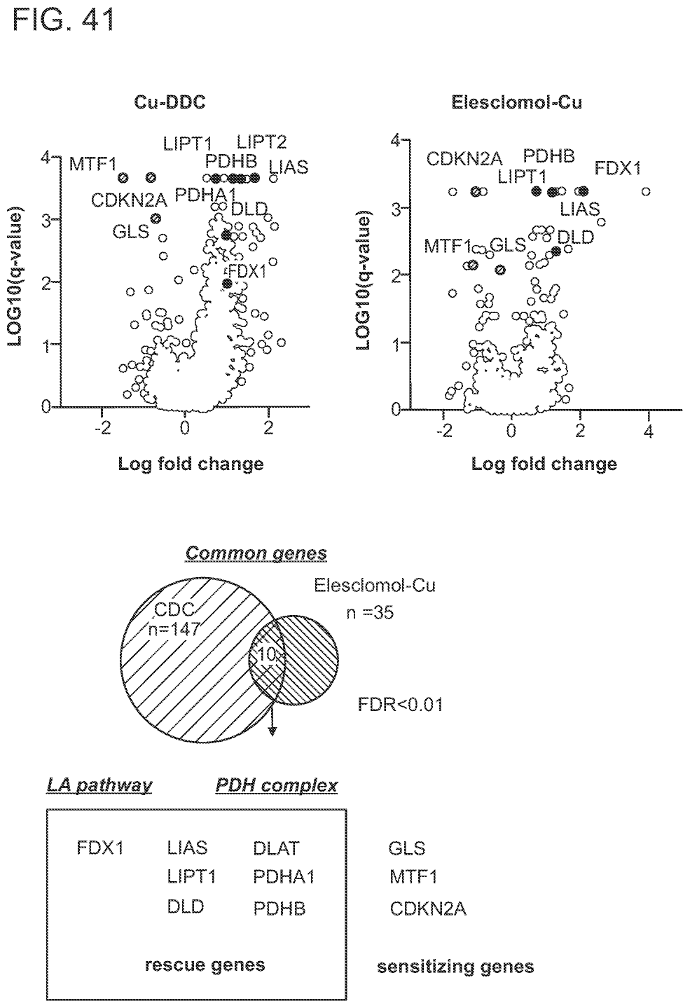

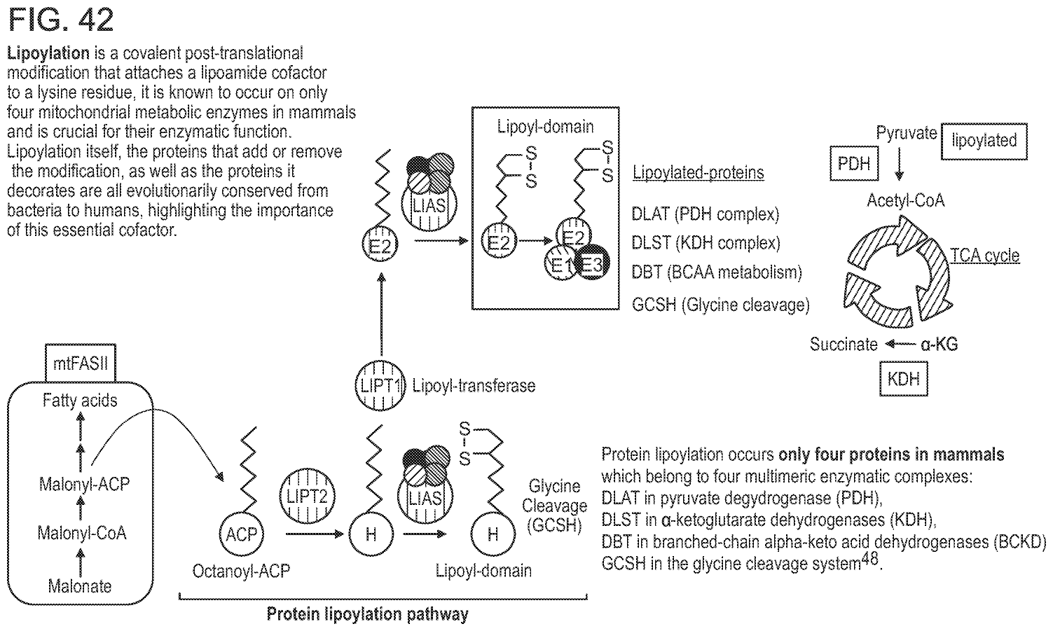

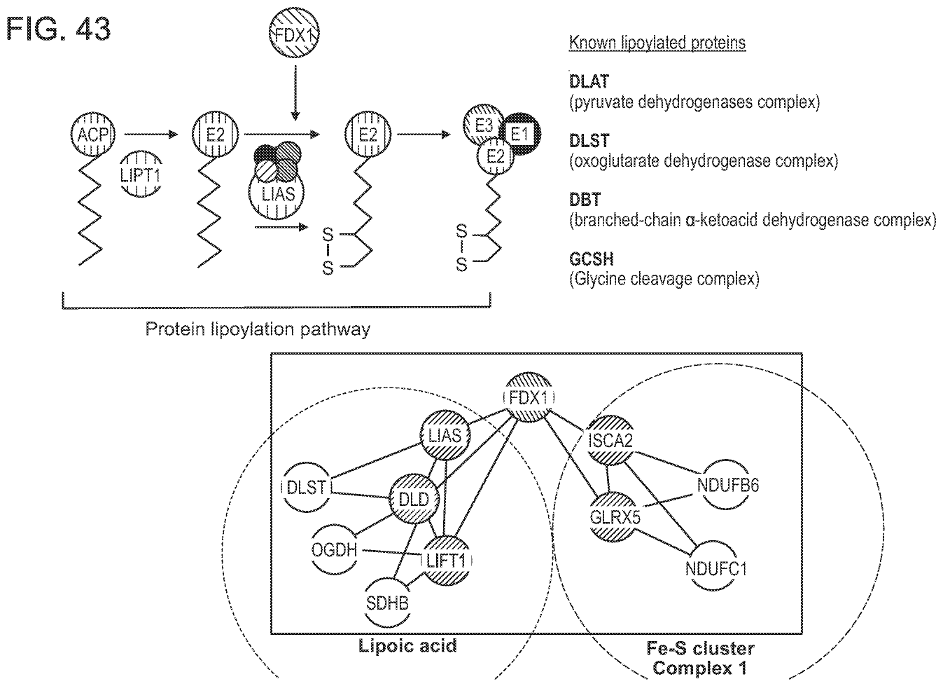

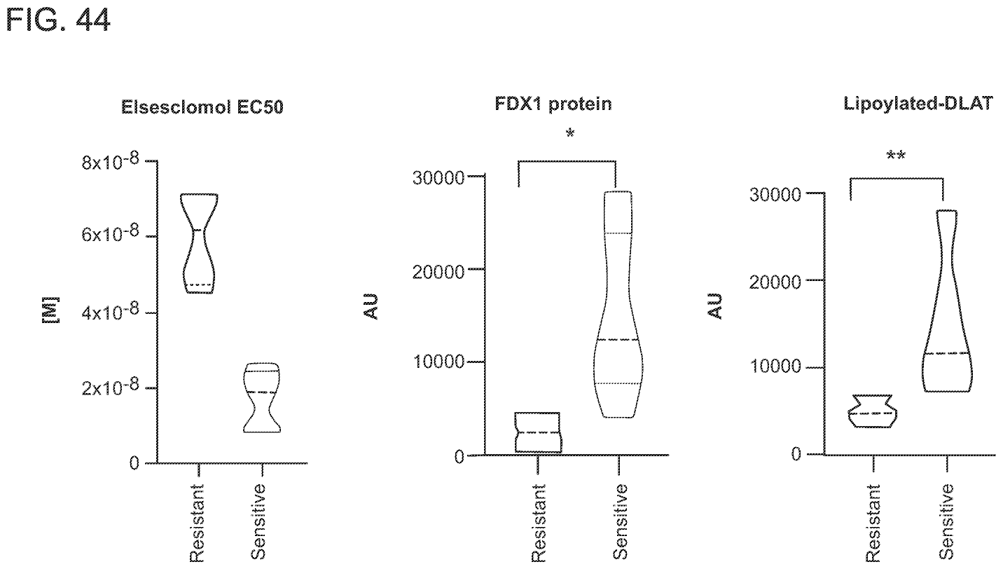

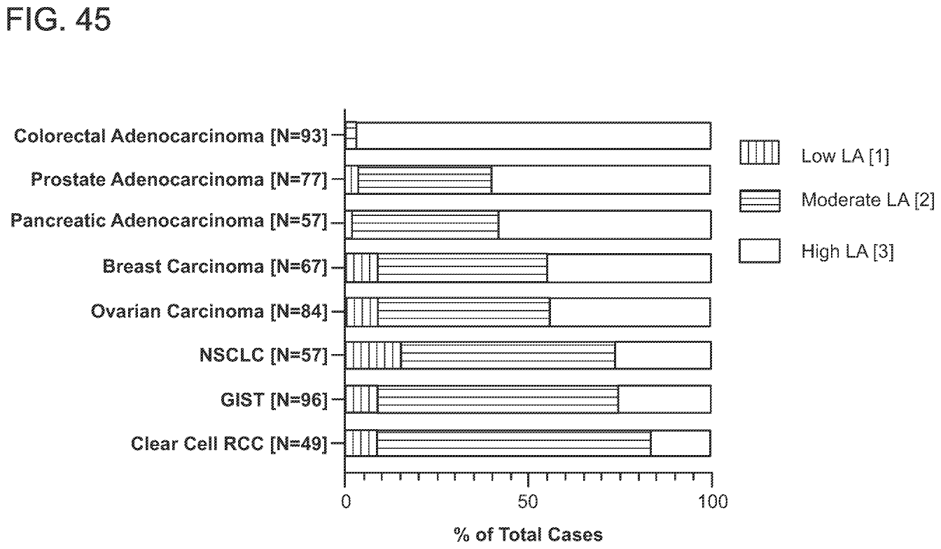

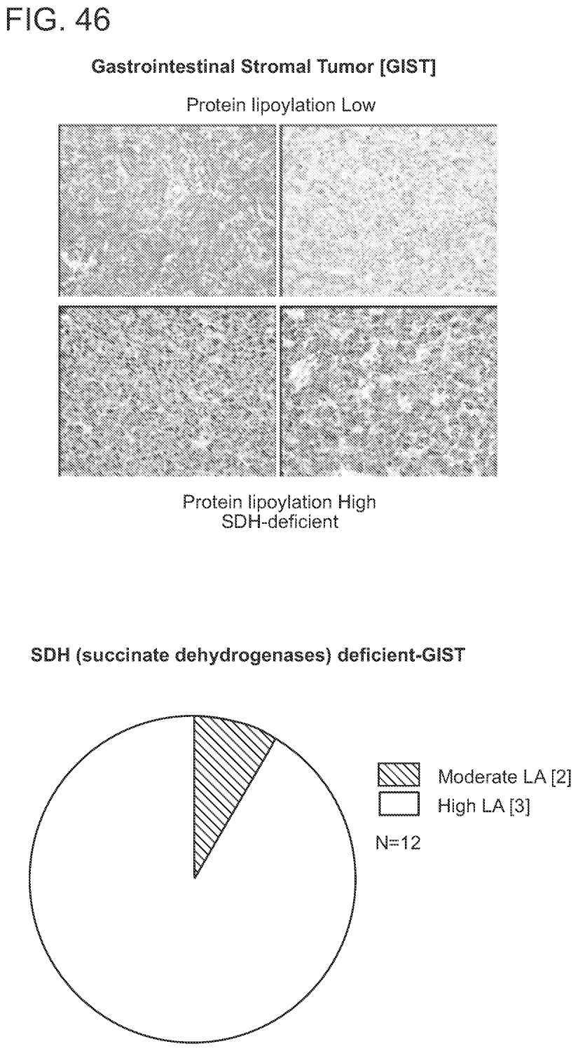

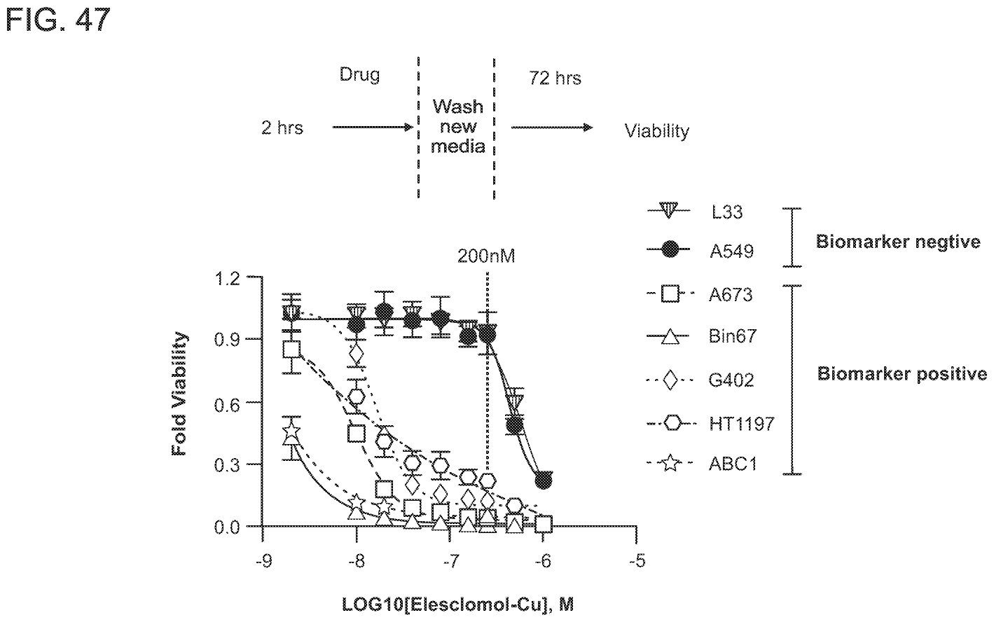

Cell lines that were shown to be either sensitive or resistant in a previous PRISM experiment were chosen to further determine the effect of elesclomol in the cell (FIG. 23). These cells on average had a higher expression of FDX1 mRNA (FIG. 26) and an overall difference in sensitivity could be reproduced (FIG. 26). The sensitive cells as a group showed overall higher levels of FDX1 protein and lipoylated protein expression levels (FIG. 27). Lipoic acid is a strong binder of copper, making it plausible that elesclomol bound copper could directly affect the lipoylated proteins. Indeed, the addition of elesclomol at concentrations as low as 10 nM reduced the levels of lipoylated proteins without dramatically affecting the protein levels (FIG. 36 and FIG. 37).

### Example 8: Viability of Cells after Increasing Doses of Copper-Binding Drugs

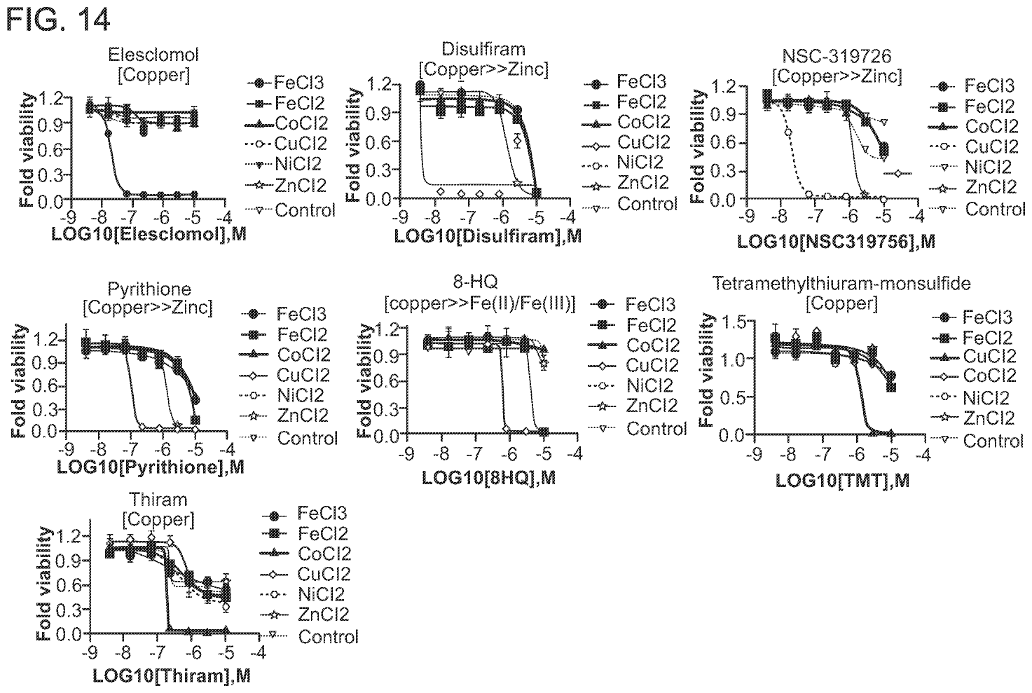

The viability of MON cells was measured following treatment with increasing doses of indicated drugs in the presence of 10 μM FeCl₂, FeCl₃, ZnCl₂, NiCl, CuCl₂, or CoCl₂ (FIG. 13). The viability of NCIH2030 cells was measured following treatment with increasing doses of indicated drugs in the presence of 10 μM FeCl₂, FeCl₃, ZnCl₂, NiCl, CuCl₂, or CoCl₂ (FIG. 14).

### Example 9: CRIPSR/Cas9 Positive Selection Screen in A549 Cells

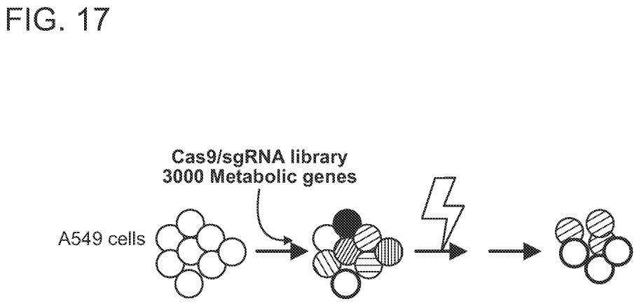

The experimental setup of the CRIPSR/Cas9 positive selection screen in A549 cells in shown in FIG. 17. The screen used a library targeting 3000 metabolism-related genes (˜10 gRNAs per gene). The most positively enriched sgRNAs in cells treated with 40 nM Elesclomol-Cu(II) of the screen are shown in Table 4.

TABLE 4

| Gene Name | Function | Number of sgRNAs |
|---|---|---|
| sgFDX1 | Fe—S Cluster | 10 |
| sgDLAT | PDH-Lipoic | 7 |
| sgNDUFB6 | Complex I | 6 |
| sgNDUFC2 | Complex I | 4 |
| sgNDUFA6 | Complex I | 4 |
| sgNDUFS1 | Complex I | 4 |
| sgISCA2 | Fe—S Cluster | 3 |
| sgLIAS | Lipoic acid | 3 |
| sgPDHB | PDH | 3 |
| sgNDUFS8 | Complex I | 3 |
| sgNDUFA2 | Complex I | 3 |
| sgNDUFS3 | Complex I | 3 |
| sgNDUFA9 | Complex I | 3 |
| sgNDUFV1 | Complex I | 3 |
| sgNDUFS2 | Complex I | 3 |
| sgNDUFB8 | Complex I | 3 |
| sgNDUFV2 | Complex I | 3 |
| sgNDUFB11 | Complex I | 3 |
| sgCNGA2 | Other | 3 |
| sgPLOD1 | Other | 3 |
| sgST6GAL2 | Other | 3 |
| sgABCA13 | Other | 3 |

Genes were sorted by functionality: Fe—S cluster pathway, lipoic acid pathway, Complex I, and other.

Deletion of FDX1 in A549 cells was found to confer relative resistance to Elesclomol-Cu(II) and disulfiram-Cu(II). Deletion of LIAS and FDX1 in OVISE cells was found to confer resistance to Elesclomol-Cu(II) (FIG. 18).

Based on results of the screen, correlation analysis revealed that FDX1 deletion correlates with the deletion of components of two distinct pathways, the lipoic acid pathway and Complex I (FIG. 19).

Deletion of FDX1 was found to eliminate cellular lipoylated proteins in both OVISE cells and K562 cells (FIG. 20 and FIG. 21).

In conclusion, a model of FDX1 function in the lipoic acid pathway is shown in FIG. 22.

### Example 10: PRISM Assay

The distribution of viability of 724 cell lines was examined by PRISM assay. FDX1 mRNA expression levels were found to be increased in the sensitive cell lines as compared to control (FIG. 23). Expression levels of FDX1 were validated in FIG. 24.

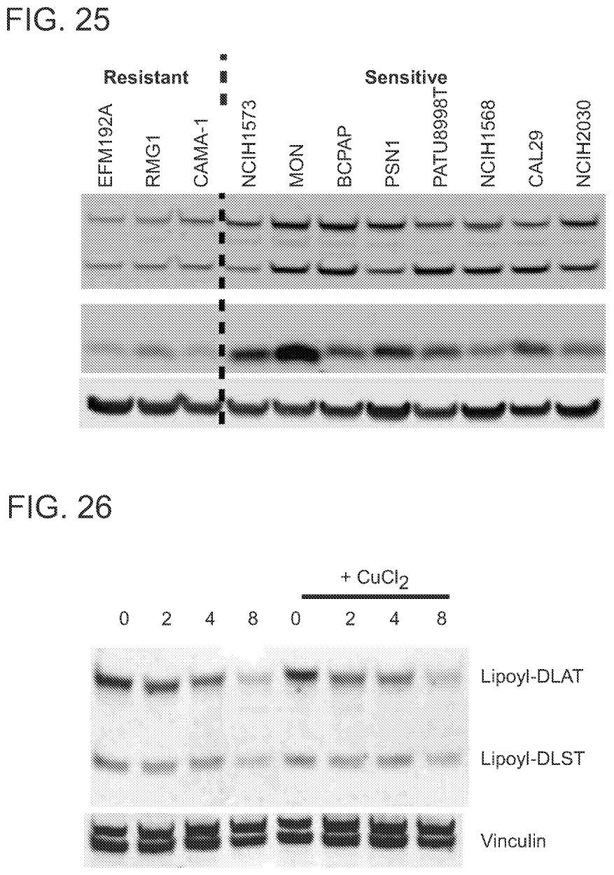

Western Blot analysis revealed that resistant cells show increased levels of FDX1 protein and lower levels of lipoylated proteins than the sensitive cells (FIG. 25). The levels of lipoylation decrease following treatment of A549 cells with 1 μM elesclomol (+CuCl₂) (FIG. 26).

### Example 11: Gene Copy Alteration Analysis

Gene copy alteration analysis was performed from The Cancer Genome Atlas (TCGA) dataset platform for biomarkers associated with elevated levels of mitochondrial metabolism. Results demonstrated that FDX1 expression is highly correlated with chromosome 11 copy number (CN) alteration (FIG. 38).

### Example 12: Establishing a Biomarker Positive Mouse Xenograft Model

Biomarker-positive cells are sensitive to elesclomol-Cu(II) treatment corresponding to measured concentrations and kinetics measured in mice pharmokinetic (PK) studies.

### Cell Culture Washout:

Study simulates short exposure time that mimics the pharmokinetic (PK) properties of elesclomol-Cu(II).

### Results:

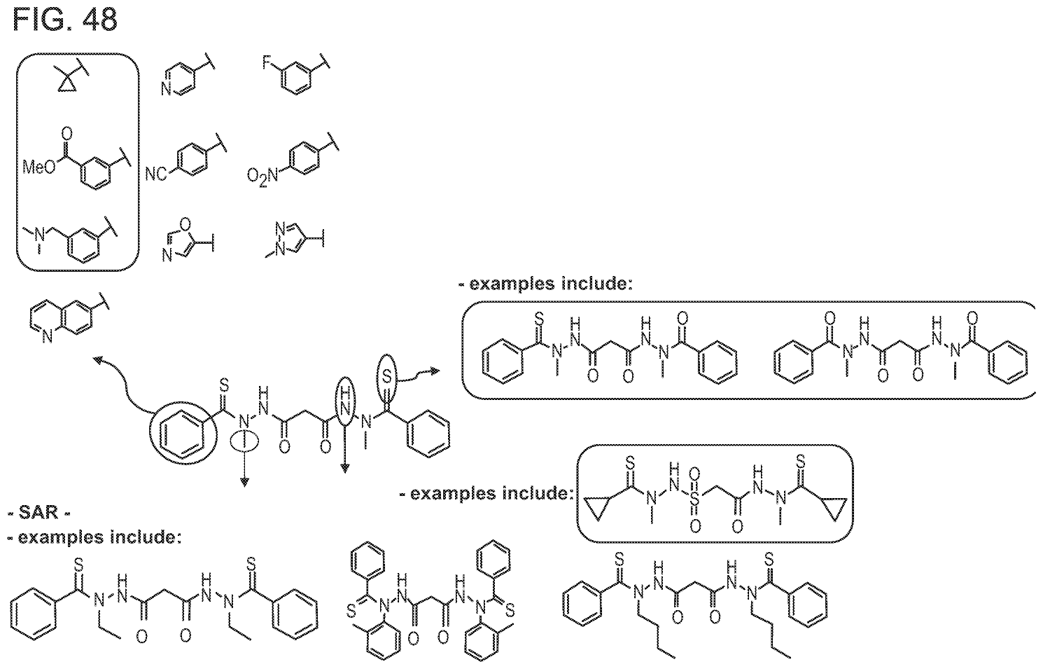

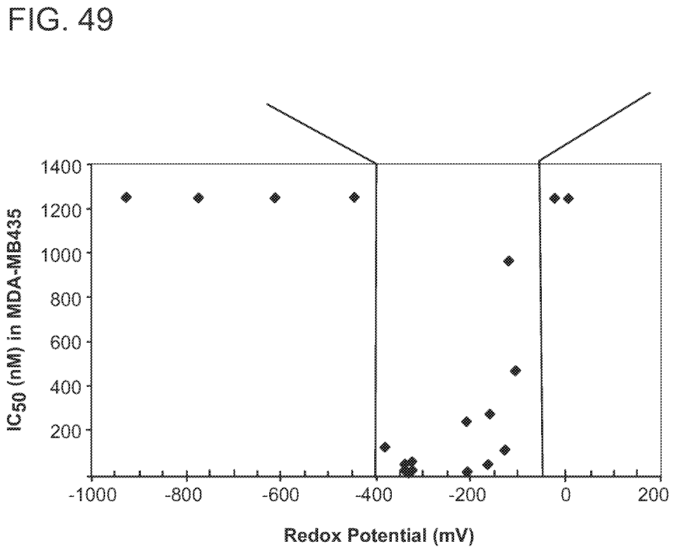

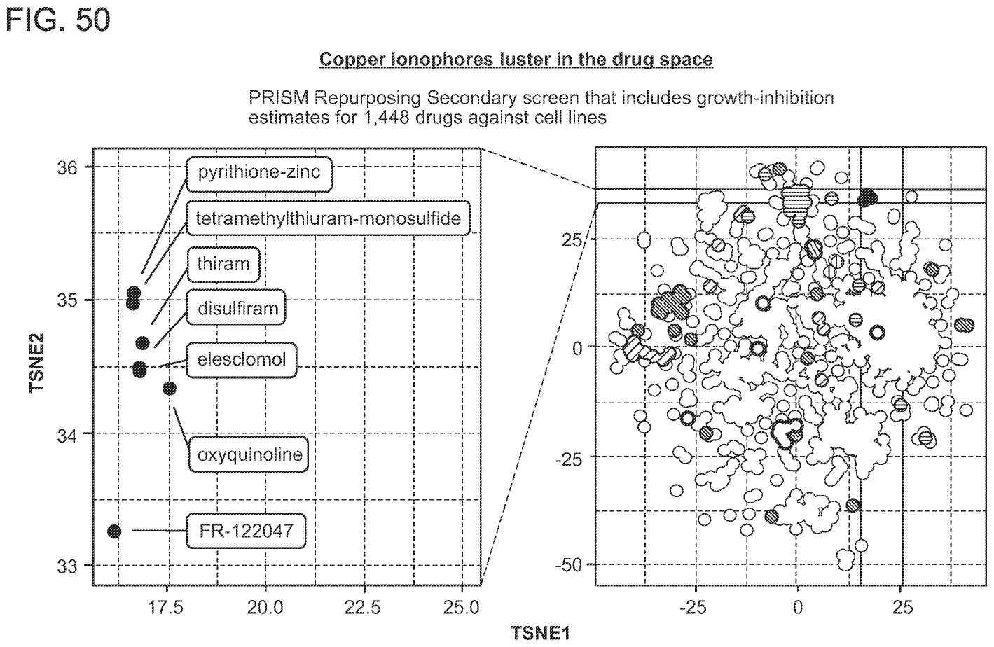

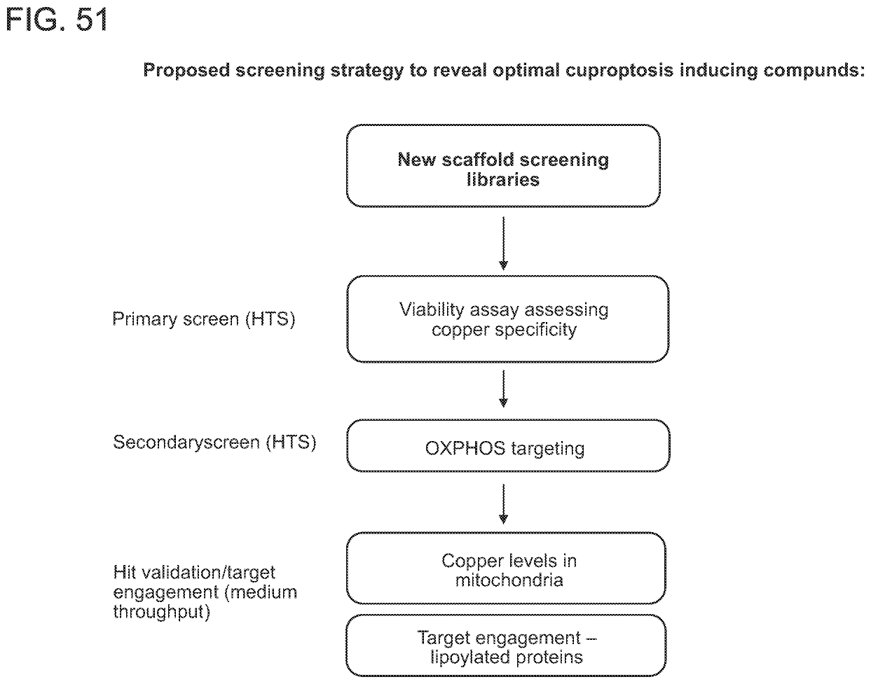

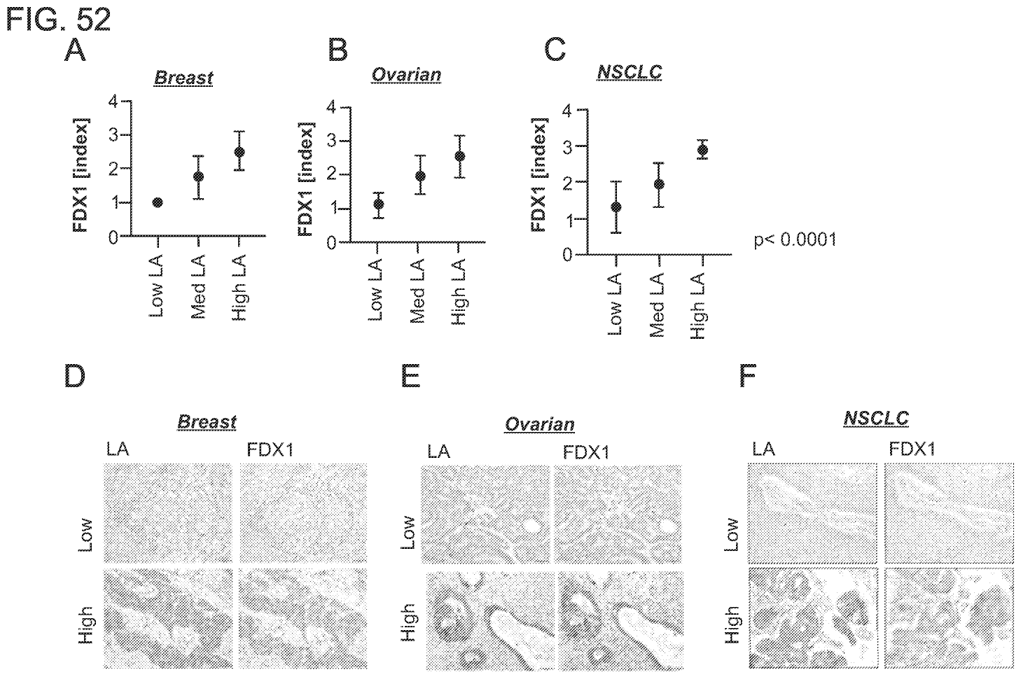

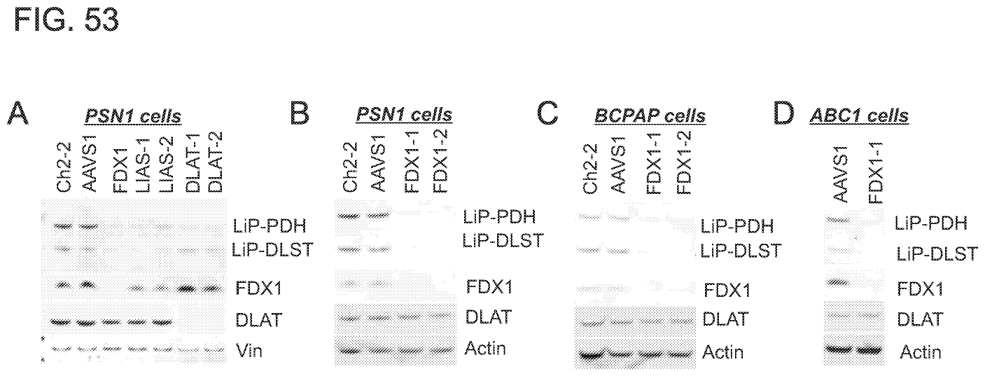

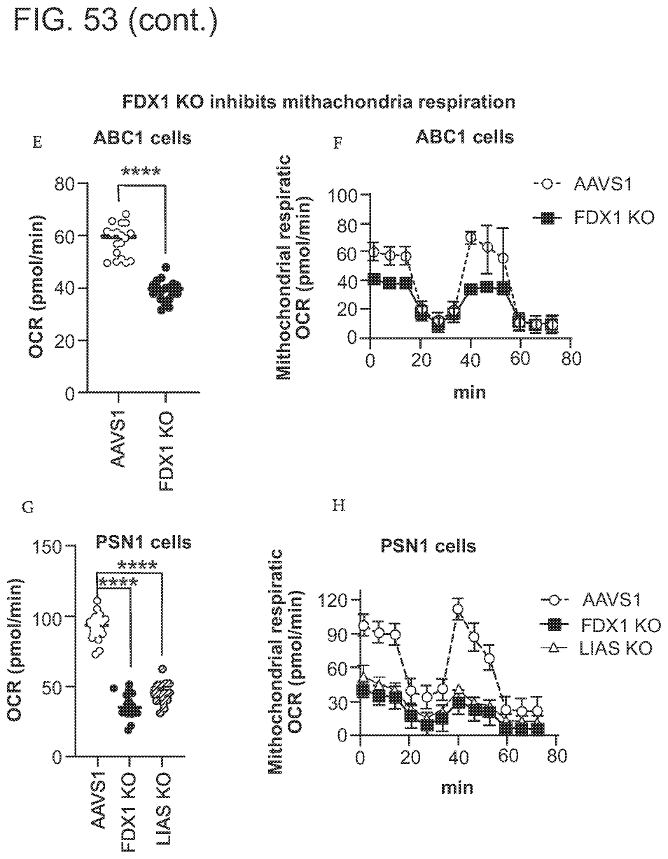

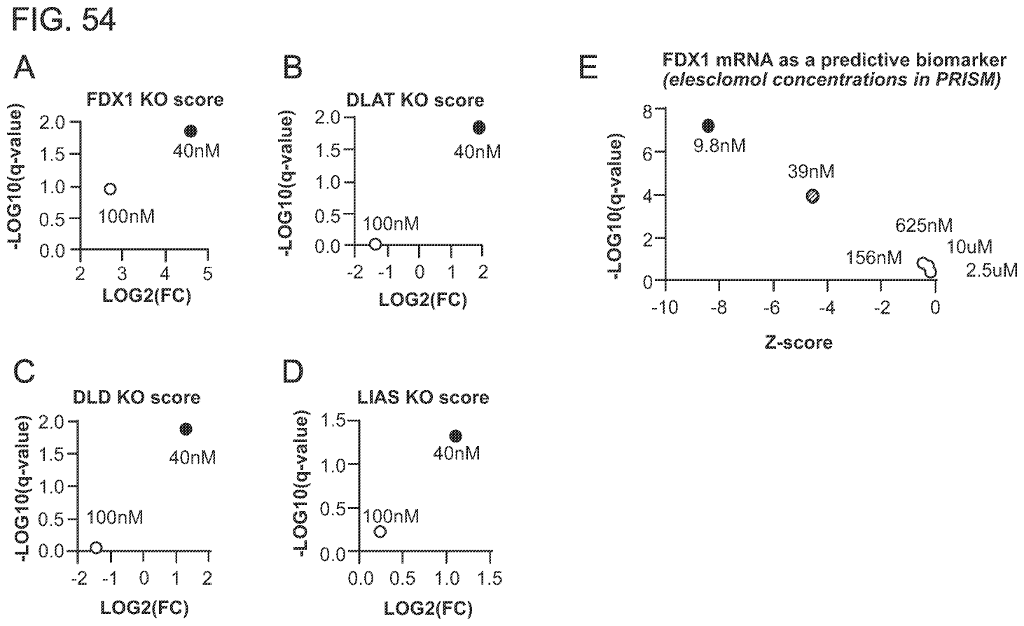

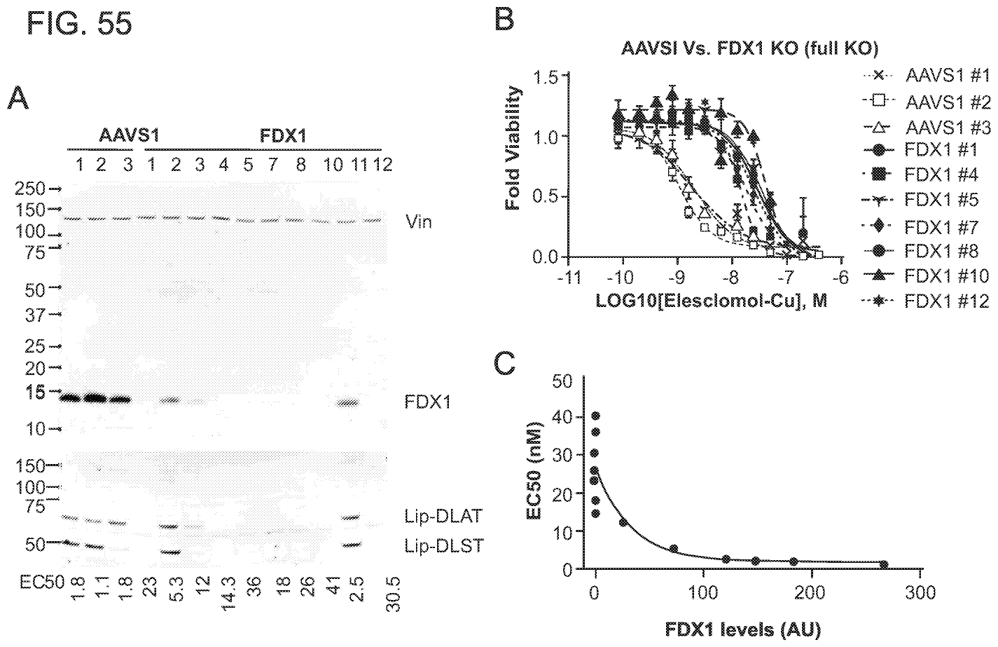

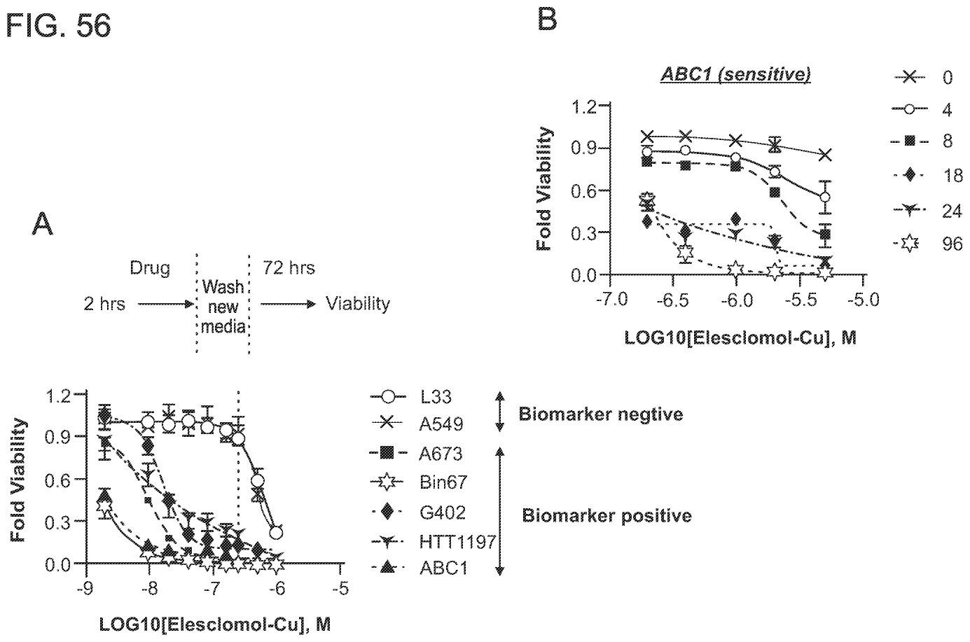

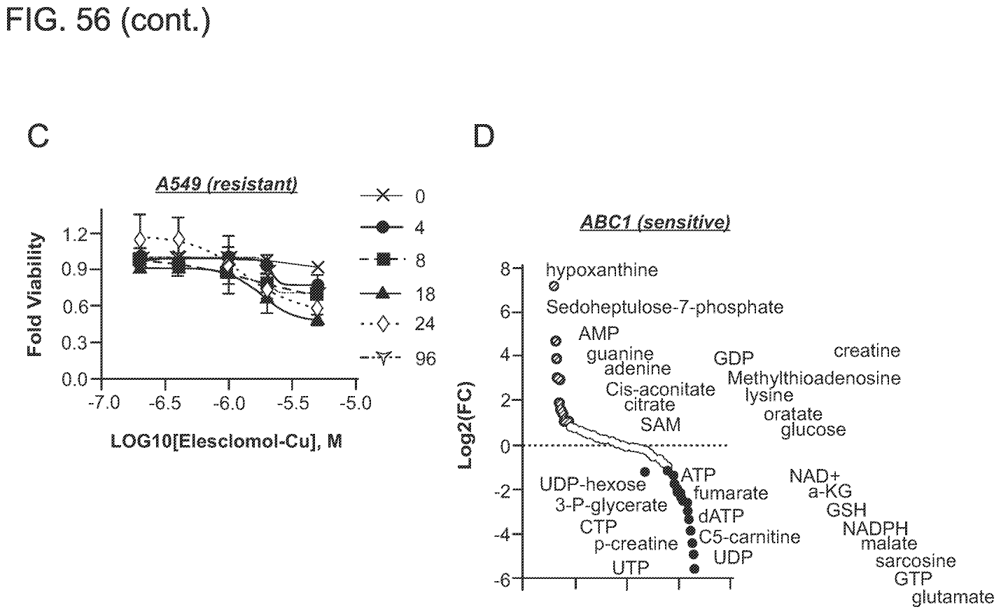

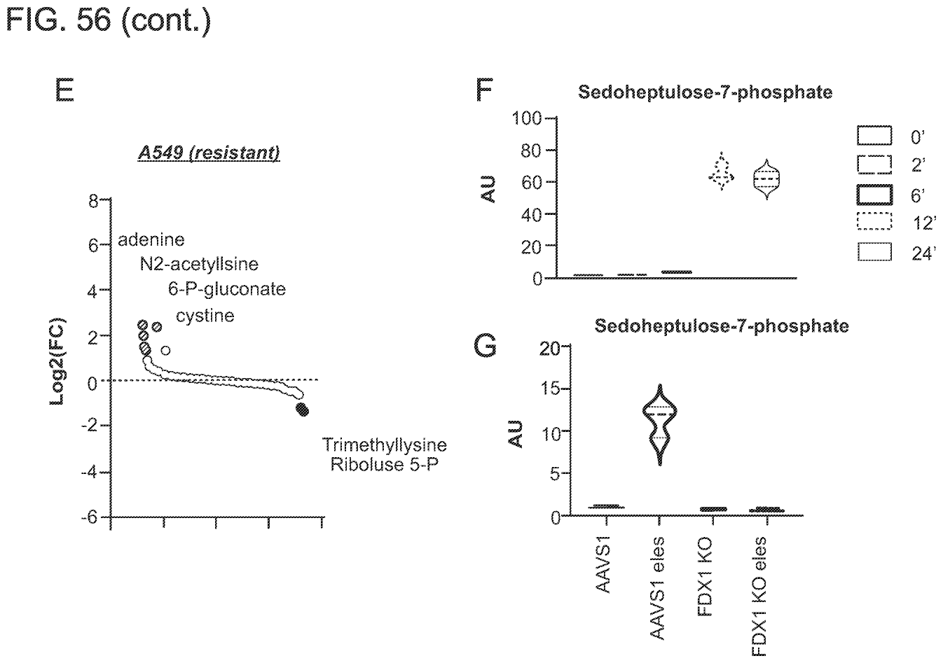

2 hour exposure at 200 nM followed by washout sufficient to effect biomarker selective cell killing. Results are shown in FIG. 48. Data supports Cₘₐₓ driven activity profile. Cells will be used to establish a SubQ xenograft model where different copper ionophores could be analyzed for their efficacy.

### EQUIVALENTS

Those skilled in the art will recognize or be able to ascertain using no more than routine experimentation, many equivalents to the specific embodiments of the invention described herein. Such equivalents are intended to be encompassed by the following claims.

What is claimed:

1. A method of inhibiting growth or proliferation of a tumor and/or immune cell, comprising:
(a) determining whether the tumor and/or immune cell comprises a level of protein lipoylation above a threshold level; and
(b) if the level of protein lipoylation is above the threshold level, contacting the tumor and/or immune cell with a copper ionophore.

2. The method of claim 1, wherein the copper ionophore induces tumor cell death and/or immune cell death.

3. The method of claim 1 or 2, wherein the lipoylated protein is lipoyl-DLAT (lipoyl-dihydrolipoamide acetyltransferase), lipoyl-DL ST (lipoyl-dihydrolipoyl succinyltransferase), lipoyl-GCSH (lipoyl-Glycine Cleavage System Protein H), or lipoyl-DBT (lipoyl-dihydrolipoamide branched chain transacylase E2).

4. The method of any one of claims 1-3, wherein determining whether the tumor and/or immune cell is characterized by a level of protein lipoylation above a threshold level comprises measuring the level of protein lipoylation in cells of the tumor and/or the immune cell.

5. The method of any one of claims 1-4, further comprising determining whether the tumor and/or immune cell is characterized by a level of a mitochondrial protein and/or a nucleic acid encoding a mitochondrial protein above a threshold level.

6. The method of claim 5, wherein determining whether the tumor and/or immune cell is characterized by a level of a mitochondrial protein and/or a nucleic acid encoding a mitochondrial protein above a threshold level comprises measuring the level of the mitochondrial protein and/or the nucleic acid encoding the mitochondrial protein in cells of the tumor and/or the immune cell.

7. The method of claim 5 or 6, wherein the mitochondrial protein binds the copper ionophore.

8. The method of any one of claims 1-7, wherein the copper ionophore is a dithiocarbamate.

9. The method of any one of claims 1-7, wherein the copper ionophore is Pyrithione Zinc.

10. The method of any one of claims 1-7, wherein the copper ionophore is Tetramethylthiuram-monosulfide.

11. The method of any one of claims 1-7, wherein the copper ionophore is Oxyquinoline (8HQ).

12. The method of any one of claims 1-7, wherein the copper ionophore is Thiram.

13. The method of any one of claims 1-7, wherein the copper ionophore is Cu(GTSM).

14. The method of any one of claims 1-7, wherein the copper ionophore is NSC-319726.

15. The method of any one of claims 1-7, wherein the copper ionophore is FR-122047.

16. The method of any one of claims 1-7, wherein the copper ionophore is Cu(isapn).

17. The method of any one of claims 1-7, wherein the copper ionophore is a Paullone-based complex.

18. The method of any one of claims 1-7, wherein the copper ionophore is a Casiopeína-based complex.

19. The method of any one of claims 1-7, wherein the copper ionophore is a Bis(thiosemicarbazone) Cu complex.

20. The method of any one of claims 1-7, wherein the copper ionophore is a Isatin-Schiff-based complex.

21. The method of any one of claims 1-7, wherein the copper ionophore is a (D-glucopyranose)-4-phenylthiosemicarbazide Cu complex.

22. The method of any one of claims 1-7, wherein the copper ionophore is a BCANa₂.

23. The method of any one of claims 1-7, wherein the copper ionophore is a BCSNa₂.

24. The method of any one of claims 1-7, wherein the copper ionophore is a BCSANa₂.

25. The method of any one of claims 1-7, wherein the copper ionophore is PTA.

26. The method of any one of claims 1-7, wherein the copper ionophore is DAPTA.

27. The method of any one of claims 1-7, wherein the copper ionophore is a soluble thiosemicarbazone complex.

28. The method of any one of claims 1-7, wherein the copper ionophore is a Schiff base complex.

29. The method of any one of claims 1-7, wherein the copper ionophore is a dithiocarbamate

30. The method of any one of claims 1-7, wherein the copper ionophore is a bis(thio-hydrazide amide).

31. The method of any one of claims 1-7, wherein the copper ionophore is a compound of Formula A or a salt thereof: 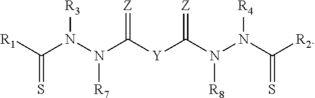
wherein:
Y is a covalent bond or an optionally substituted straight chained hydrocarbyl group, or, Y, taken together with both >C═Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic ring optionally fused to an aromatic ring;
R₇ and R₈ are independently —H, an optionally substituted aliphatic group, or an optionally substituted aryl group; and
Z is O or S.

32. The method of any one of claims 1-7, wherein the copper ionophore is a compound of Formula B1 or a salt thereof: 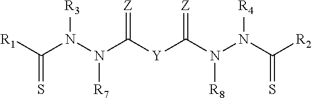
wherein:
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic ring optionally fused to an aromatic ring;
R₇ and R₈ are independently —H, an optionally substituted aliphatic group, or an optionally substituted aryl group; and
Z is O or S.

33. The method of any one of claims 1-7, wherein the copper ionophore is a compound of Formula B2 or a salt thereof: 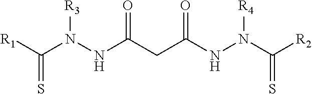
wherein:
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic ring optionally fused to an aromatic ring.

34. The method of any one of claims 1-7, wherein the copper ionophore is a compound of Formula C or a salt thereof: 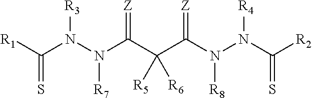
wherein:
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic ring optionally fused to an aromatic ring;
R₅ and R₆ are independently —H or lower alkyl;
R₇ and R₈ are independently —H, an optionally substituted aliphatic group, or an optionally substituted aryl group; and
Z is O or S.

35. The method of any one of claims 1-7, wherein the copper ionophore is a compound of Formula D or a salt thereof: 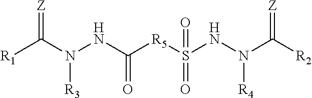
wherein:
each Z is independently S, O or Se, provided that Z cannot both be O;
R₁ and R₂ are each independently selected from the group consisting of an optionally substituted alkyl, an optionally substituted alkenyl, an optionally substituted alkynyl; an optionally substituted cycloalkyl, an optionally substituted cycloalkenyl, an optionally substituted heterocyclic group wherein the heterocyclic group is bonded to the thiocarbonyl carbon via a carbon-carbon linkage, an optionally substituted phenyl, an optionally substituted bicyclic aryl, an optionally substituted five to seven-membered monocyclic heteroaryl, an optionally substituted nine to fourteen-membered bicyclic heteroaryl wherein the heteroaryl group is bonded to the thiocarbonyl carbon via a carbon-carbon linkage, —NR₁₂R₁₃, —OR₁₄, —SR₁₄ and —S(O)ₚR₁₅;
R₃ and R₄ are each independently selected from the group consisting of hydrogen, an optionally substituted alkyl, an optionally substituted alkenyl, an optionally substituted alkynyl, an optionally substituted cycloalkyl, an optionally substituted cycloalkenyl, an optionally substituted heterocyclic group, and an optionally substituted five to six-membered aryl or heteroaryl group; or
R₁ and R₃ and/or R₂ and R₄, taken together with the atoms to which they are attached, form an optionally substituted heterocyclic group or an optionally substituted heteroaryl group;
R₅ is —CR₆R₇—, —C(═CHR₈)— or —C(═NR₈)—;
R₆ and R₇ are both —H or an optionally substituted lower alkyl;
R₈ is selected from the group consisting of —OH, an alkyl, an alkenyl, an alkynyl, an alkoxy, an alkenoxy, an alkynoxyl, a hydroxyalkyl, a hydroxyalkenyl, a hydroxyalkynyl, a haloalkyl, a haloalkenyl, a haloalkynyl, an optionally substituted phenyl, an optionally substituted bicyclic aryl, an optionally substituted five to six-membered monocyclic heteroaryl, an optionally substituted nine to fourteen-membered bicyclic heteroaryl, an optionally substituted cycloalkyl or an optionally substituted heterocyclic group, —NR₁₀R₁₁, and —COR₉;
R₉ is an optionally substituted phenyl, an optionally substituted bicyclic aryl, an optionally substituted five or six-membered monocyclic heteroaryl, an optionally substituted nine to fourteen-membered bicyclic heteroaryl, an optionally substituted alkyl, an optionally substituted cycloalkyl or an optionally substituted heterocyclic group;
R₁₀ and R₁₁ are each independently selected from the group consisting of —H, —OH, amino, (di)alkylamino, an alkyl, an alkenyl, an alkynyl, an alkoxy, an alkenoxy, an alkynoxyl, a hydroxyalkyl, a hydroxyalkenyl, a hydroxyalkynyl, a haloalkyl, a haloalkenyl, a haloalkynyl, an optionally substituted phenyl, an optionally substituted bicyclic aryl, an optionally substituted five to six-membered monocyclic heteroaryl, an optionally substituted nine to fourteen-membered bicyclic heteroaryl, an optionally substituted cycloalkyl or an optionally substituted heterocyclic group and —COR₉, or R₁₀ and R₁₁, taken together with the nitrogen atom to which they are attached, form a five to six-membered heteroaryl group; and
R₁₂, R₁₃ and R₁₄ are each independently —H, an optionally substituted alkyl, an optionally substituted phenyl or an optionally substituted benzyl, or R₁₂ and R₁₃, taken together with the nitrogen atom to which they are attached, form an optionally substituted heterocyclic group or an optionally substituted heteroaryl group;
R₁₅ is an optionally substituted alkyl, an optionally substituted aryl or an optionally substituted heteroaryl, and
p is 1 or 2;
provided that when both Z are S and R₃ and R₄ are both methyl, then R₁ and R₂ are not both unsubstituted phenyl.

36. The method of any one of claims 1-7, wherein the copper ionophore is a compound of Formula E or a salt thereof: 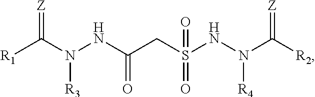
wherein:
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic ring optionally fused to an aromatic ring;

37. The method of any one of claims 1-7, wherein the copper ionophore is a compound of the following formula or a salt thereof: 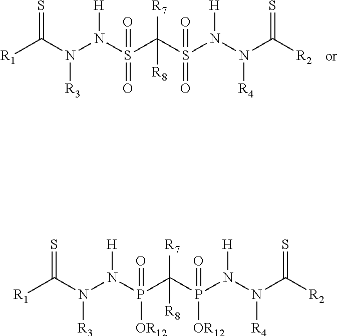
wherein:
R₁ and R₂ are independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, halo, nitro, cyano, guanidino, —OR₁₇, —NR₁₉R₂₀, —C(O)R₁₇, —C(O)OR₁₇, —OC(O)R₁₇, —C(O)NR₁₉R₂₀, —NR₁₈C(O)R₁₇, —OP(O)(OR₁₇)₂, —SP(O)(OR₁₇)₂, —SR₁₇, —S(O)ₚR₁₇, —OS(O)ₚR₁₇, —S(O)ₚOR₁₇, —NR₁₈S(O)ₚR₁₇, or —S(O)ₚNR₁₉R₂₀;
R₃ and R₄ are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl or an optionally substituted heteroaryl,
R₇ and R₈ are each independently —H or an optionally substituted alkyl, an optionally substituted alkenyl, an optionally substituted alkynyl, an optionally substituted cycloalkyl, an optionally substituted cycloalkenyl, an optionally substituted heterocyclyl, or R₇ is —H and R₈ is an optionally substituted aryl or an optionally substituted heteroaryl, and R₁, R₂, R₃; and
R₁₂ is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, or halo.

38. The method of any one of claims 1-7, wherein the copper ionophore is an ALDH inhibitor.

39. The method of any one of claims 1-7, wherein the copper ionophore is elesclomol.

40. The method of any one of claim 5-7 or 39, wherein the mitochondrial protein is FDX1 (ferredoxin 1).

41. The method of any one of claims 1-7, wherein the copper ionophore is disulfiram.

42. The method of any one of claim 1-7 or 10, wherein the mitochondrial protein is ALDHA1 (aldehyde dehydrogenase A1) or ALDH2 (aldehyde dehydrogenase 2).

43. The method of claim 5 or 6, wherein the mitochondrial protein is a protein involved in lipoic acid biosynthesis.

44. The method of claim 5, 6, or 43, wherein the protein involved in lipoic acid biosynthesis is LIAS (lipoic acid synthetase), LIPT1 (lipoyltransferase 1), or LIPT2 (lipoyltransferase 2), or DLD (Dihydrolipoamide Dehydrogenase).

45. The method of any one of claims 1-44, wherein contacting the tumor and/or immune cell with the copper ionophore inhibits Pyruvate dehydrogenase complex, 2-oxoglutarate dehydrogenase complex, Branched-Chain Alpha-Keto Acid Dehydrogenase Complex, and/or glycine cleavage.

46. The method of any one of claims 1-45, further comprising treating the tumor and/or immune cell with another anti-cancer agent conjointly with the copper ionophore.

47. The method of claim 46, whereby the copper ionophore enhances the effects of the anti-cancer agent relative to the anti-cancer agent alone.

48. The method of claim 46 or 47, wherein the anti-cancer agent is a chemotherapeutic agent, an immune checkpoint inhibitor, an EGFR inhibitor, or a proteasome inhibitor.

49. The method of claim 48, wherein the anti-cancer agent is a chemotherapeutic agent.

50. The method of claim 49, wherein the chemotherapeutic agent is cytarabine.

51. The method of claim 49, wherein the chemotherapeutic agent is a b-raf inhibitor.

52. The method of claim 49, wherein the chemotherapeutic agent is docetaxel.

53. The method of claim 49, wherein the chemotherapeutic agent is imatinib.

54. The method of claim 48, wherein the anti-cancer agent is an EGFR inhibitor.

55. The method of claim 54, wherein the EGFR inhibitor is a tyrosine kinase inhibitor.

56. The method of claim 54, wherein the EGFR inhibitor is gefitinib.

57. The method of claim 54, wherein the EGFR inhibitor is osimertinib.

58. The method of any one of claims 46-27, wherein the copper ionophore enhances tumor cell death and/or immune cell death of the anti-cancer agent relative to the anti-cancer agent alone.

59. The method of any one of claims 1-58, wherein the copper ionophore is pre-loaded with copper(II).

60. The method of claim 59, wherein the copper ionophore is elesclomol.

61. The method of claim 59, wherein the copper ionophore is disulfiram.

62. A method of treating cancer refractory to treatment with an anti-cancer agent in a subject, comprising the steps of:
(a) determining whether the cancer is characterized by a level of protein lipoylation above a threshold level; and
(b) if the cancer is characterized by a level of protein lipoylation above the threshold level, conjointly administering a copper ionophore and the anti-cancer agent to the subject.

63. The method of claim 62, wherein the anti-cancer agent is a chemotherapeutic agent, an immune checkpoint inhibitor, an EGFR inhibitor, or a proteasome inhibitor.

64. The method of claim 63, wherein the anti-cancer agent is a chemotherapeutic agent.

65. The method of claim 64 wherein the chemotherapeutic agent is cytarabine.

66. The method of claim 64, wherein the chemotherapeutic agent is a b-raf inhibitor.

67. The method of claim 64, wherein the chemotherapeutic agent is docetaxel.

68. The method of claim 64, wherein the chemotherapeutic agent is imatinib.

69. The method of claim 63, wherein the anti-cancer agent is an EGFR inhibitor.

70. The method of claim 69, wherein the EGFR inhibitor is a tyrosine kinase inhibitor.

71. The method of claim 69, wherein the EGFR inhibitor is gefitinib.

72. The method of claim 69, wherein the EGFR inhibitor is osimertinib.

73. The method of any one of claims 62-72, wherein the lipoylated protein is lipoyl-DLAT, lipoyl-DLST, lipoyl-GCSH, or lipoyl-DBT.

74. The method of any one of claims 62-73, wherein determining whether the cancer is characterized by a level of protein lipoylation above a threshold level comprises measuring the level of protein lipoylation in cells of the cancer.

75. The method of any one of claims 62-74, further comprising determining whether the cancer is characterized by a level of a mitochondrial protein and/or a nucleic acid encoding a mitochondrial protein above a threshold level.

76. The method of any one of claim 75, wherein determining whether the cancer is characterized by a level of a mitochondrial protein and/or a nucleic acid encoding a mitochondrial protein above a threshold level comprises measuring the level of the mitochondrial protein and/or the nucleic acid encoding the mitochondrial protein in the cells of the cancer.

77. The method of claim 42 or 43, wherein the copper ionophore binds the mitochondrial protein.

78. The method of any one of claims 62-77, wherein the copper ionophore is a dithiocarbamate.

79. The method of any one of claims 62-77, wherein the copper ionophore is Pyrithione Zinc.

80. The method of any one of claims 62-77, wherein the copper ionophore is Tetramethylthiuram-monosulfide.

81. The method of any one of claims 62-77, wherein the copper ionophore is Oxyquinoline (8HQ).

82. The method of any one of claims 62-77, wherein the copper ionophore is Thiram.

83. The method of any one of claims 62-77, wherein the copper ionophore is Cu(GTSM).

84. The method of any one of claims 62-77, wherein the copper ionophore is NSC-319726.

85. The method of any one of claims 62-77, wherein the copper ionophore is FR-122047.

86. The method of any one of claims 62-77, wherein the copper ionophore is Cu(isapn).

87. The method of any one of claims 62-77, wherein the copper ionophore is a Paullone-based complex.

88. The method of any one of claims 62-77, wherein the copper ionophore is a Casiopeína-based complex.

89. The method of any one of claims 62-77, wherein the copper ionophore is a Bis(thiosemicarbazone) Cu complex.

90. The method of any one of claims 62-77, wherein the copper ionophore is a Isatin-Schiff-based complex.

91. The method of any one of claims 62-77, wherein the copper ionophore is a (D-glucopyranose)-4-phenylthiosemicarbazide Cu complex.

92. The method of any one of claims 62-77, wherein the copper ionophore is a BCANa₂.

93. The method of any one of claims 62-77, wherein the copper ionophore is a BCSNa₂.

94. The method of any one of claims 62-77, wherein the copper ionophore is a BCSANa₂.

95. The method of any one of claims 62-77, wherein the copper ionophore is PTA.

96. The method of any one of claims 62-77, wherein the copper ionophore is DAPTA.

97. The method of any one of claims 62-77, wherein the copper ionophore is a soluble thiosemicarbazone complex.

98. The method of any one of claims 62-77, wherein the copper ionophore is a Schiff base complex.

99. The method of any one of claims 62-77, wherein the copper ionophore is a dithiocarbamate

100. The method of any one of claims 62-77, wherein the copper ionophore is a bis(thio-hydrazide amide).

101. The method of any one of claims 62-77, wherein the copper ionophore is a compound of Formula A or a salt thereof: 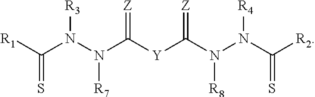
wherein:
Y is a covalent bond or an optionally substituted straight chained hydrocarbyl group, or, Y, taken together with both >C═Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic ring optionally fused to an aromatic ring;
R₇ and R₈ are independently —H, an optionally substituted aliphatic group, or an optionally substituted aryl group; and
Z is O or S.

102. The method of any one of claims 62-77, wherein the copper ionophore is a compound of Formula B1 or a salt thereof: 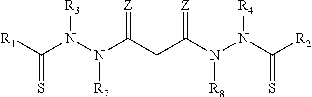
wherein:
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic ring optionally fused to an aromatic ring;
R₇ and R₈ are independently —H, an optionally substituted aliphatic group, or an optionally substituted aryl group; and
Z is O or S.

103. The method of any one of claims 62-77, wherein the copper ionophore is a compound of Formula B2 or a salt thereof: 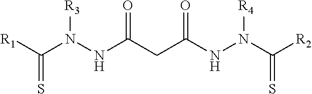
wherein:
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic ring optionally fused to an aromatic ring.

104. The method of any one of claims 62-77, wherein the copper ionophore is a compound of Formula C or a salt thereof: 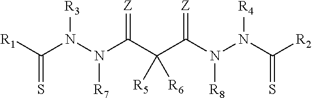
wherein:
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic ring optionally fused to an aromatic ring;
R₅ and R₆ are independently —H or lower alkyl;
R₇ and R₈ are independently —H, an optionally substituted aliphatic group, or an optionally substituted aryl group; and
Z is O or S.

105. The method of any one of claims 62-77, wherein the copper ionophore is a compound of Formula D or a salt thereof: 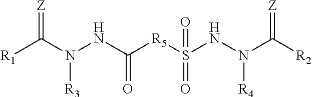
wherein:
each Z is independently S, O or Se, provided that Z cannot both be O;
R₁ and R₂ are each independently selected from the group consisting of an optionally substituted alkyl, an optionally substituted alkenyl, an optionally substituted alkynyl; an optionally substituted cycloalkyl, an optionally substituted cycloalkenyl, an optionally substituted heterocyclic group wherein the heterocyclic group is bonded to the thiocarbonyl carbon via a carbon-carbon linkage, an optionally substituted phenyl, an optionally substituted bicyclic aryl, an optionally substituted five to seven-membered monocyclic heteroaryl, an optionally substituted nine to fourteen-membered bicyclic heteroaryl wherein the heteroaryl group is bonded to the thiocarbonyl carbon via a carbon-carbon linkage, —NR₁₂R₁₃, —OR₁₄, —SR₁₄ and —S(O)ₚR₁₅;
R₃ and R₄ are each independently selected from the group consisting of hydrogen, an optionally substituted alkyl, an optionally substituted alkenyl, an optionally substituted alkynyl, an optionally substituted cycloalkyl, an optionally substituted cycloalkenyl, an optionally substituted heterocyclic group, and an optionally substituted five to six-membered aryl or heteroaryl group; or
R₁ and R₃ and/or R₂ and R₄, taken together with the atoms to which they are attached, form an optionally substituted heterocyclic group or an optionally substituted heteroaryl group;
R₅ is —CR₆R₇—, —C(═CHR₈)— or —C(═NR₈)—;
R₆ and R₇ are both —H or an optionally substituted lower alkyl;
R₈ is selected from the group consisting of —OH, an alkyl, an alkenyl, an alkynyl, an alkoxy, an alkenoxy, an alkynoxyl, a hydroxyalkyl, a hydroxyalkenyl, a hydroxyalkynyl, a haloalkyl, a haloalkenyl, a haloalkynyl, an optionally substituted phenyl, an optionally substituted bicyclic aryl, an optionally substituted five to six-membered monocyclic heteroaryl, an optionally substituted nine to fourteen-membered bicyclic heteroaryl, an optionally substituted cycloalkyl or an optionally substituted heterocyclic group, —NR₁₀R₁₁, and —COR₉;
R₉ is an optionally substituted phenyl, an optionally substituted bicyclic aryl, an optionally substituted five or six-membered monocyclic heteroaryl, an optionally substituted nine to fourteen-membered bicyclic heteroaryl, an optionally substituted alkyl, an optionally substituted cycloalkyl or an optionally substituted heterocyclic group;
R₁₀ and R₁₁ are each independently selected from the group consisting of —H, —OH, amino, (di)alkylamino, an alkyl, an alkenyl, an alkynyl, an alkoxy, an alkenoxy, an alkynoxyl, a hydroxyalkyl, a hydroxyalkenyl, a hydroxyalkynyl, a haloalkyl, a haloalkenyl, a haloalkynyl, an optionally substituted phenyl, an optionally substituted bicyclic aryl, an optionally substituted five to six-membered monocyclic heteroaryl, an optionally substituted nine to fourteen-membered bicyclic heteroaryl, an optionally substituted cycloalkyl or an optionally substituted heterocyclic group and —COR₉, or R₁₀ and R₁₁, taken together with the nitrogen atom to which they are attached, form a five to six-membered heteroaryl group; and
R₁₂, R₁₃ and R₁₄ are each independently —H, an optionally substituted alkyl, an optionally substituted phenyl or an optionally substituted benzyl, or R₁₂ and R₁₃, taken together with the nitrogen atom to which they are attached, form an optionally substituted heterocyclic group or an optionally substituted heteroaryl group;
R₁₅ is an optionally substituted alkyl, an optionally substituted aryl or an optionally substituted heteroaryl, and
p is 1 or 2;
provided that when both Z are S and R₃ and R₄ are both methyl, then R₁ and R₂ are not both unsubstituted phenyl.

106. The method of any one of claims 62-77, wherein the copper ionophore is a compound of Formula E or a salt thereof: 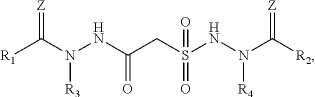
wherein:
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic ring optionally fused to an aromatic ring;

107. The method of any one of claims 62-77, wherein the copper ionophore is a compound of the following formula or a salt thereof: 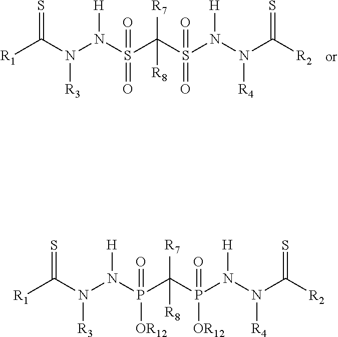
wherein:
R₁ and R₂ are independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, halo, nitro, cyano, guanidino, —OR₁₇, —NR₁₉R₂₀, —C(O)R₁₇, —C(O)OR₁₇, —OC(O)R₁₇, —C(O)NR₁₉R₂₀, —NR₁₈C(O)R₁₇, —OP(O)(OR₁₇)₂, —SP(O)(OR₁₇)₂, —SR₁₇, —S(O)ₚR₁₇, —OS(O)ₚR₁₇, —S(O)ₚOR₁₇, —NR₁₈S(O)ₚR₁₇, or —S(O)ₚNR₁₉R₂₀;
R₃ and R₄ are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl or an optionally substituted heteroaryl;
R₇ and R₈ are each independently —H or an optionally substituted alkyl, an optionally substituted alkenyl, an optionally substituted alkynyl, an optionally substituted cycloalkyl, an optionally substituted cycloalkenyl, an optionally substituted heterocyclyl, or R₇ is —H and R₈ is an optionally substituted aryl or an optionally substituted heteroaryl; and R₁, R₂, R₃; and
R₁₂ is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, or halo.

108. The method of any one of claims 62-77, wherein the copper ionophore is an ALDH inhibitor.

109. The method of any one of claims 62-77, wherein the copper ionophore is elesclomol.

110. The method of any one of claim 75-77 or 109, wherein the mitochondrial protein is FDX1.

111. The method of any one of claims 62-77, wherein the copper ionophore is disulfiram.

112. The method of any one of claim 75-77 or 111, wherein the mitochondrial protein is ALDHA1 or ALDH2.

113. The method of claim 75 or 76, wherein the mitochondrial protein is a protein involved in lipoic acid biosynthesis.

114. The method of claim 75, 76, or 113, wherein the protein involved in lipoic acid biosynthesis is LIAS, LIPT1, LIPT2, or DLD.

115. The method of any one of claims 62-114, wherein contacting the tumor and/or immune cell with the copper ionophore inhibits Pyruvate dehydrogenase complex, 2-oxoglutarate dehydrogenase complex, Branched-Chain Alpha-Keto Acid Dehydrogenase Complex, and/or glycine cleavage.

116. The method of any one of claims 62-115, wherein the copper ionophore is pre-loaded with copper(II).

117. The method of claim 116, wherein the copper ionophore is elesclomol.

118. The method of claim 116, wherein the copper ionophore is disulfiram.

119. A method of identifying a candidate anti-cancer agent, comprising the steps of:
(a) contacting a cell sample with a test agent;
(b) measuring a level of cellular protein lipoylation of the cell sample; and
(c) identifying the test agent as a candidate anti-cancer agent if the level of cellular protein lipoylation is decreased as compared to a level of cellular protein lipoylation of a cell sample not contacted with the test agent.

120. The method of claim 119, wherein the level of cellular protein lipoylation of a cell sample not contacted with the test agent is the level of cellular protein lipoylation in the cell sample prior to contact with the test agent.

121. The method of claim 119 or 120, wherein the level of cellular protein lipoylation of a cell sample not contacted with the test agent is the level of cellular protein lipoylation of a corresponding control cell sample.

122. The method of any one of claims 119-121, wherein the level of cellular protein lipoylation of a cell sample not contacted with the test agent is the level of cellular protein lipoylation of one or more reference samples representative of the cell sample contacted with the test agent.

123. The method of any one of claims 119-122, wherein the lipoylated protein is lipoyl-DLAT, lipoyl-DLST, lipoyl-GCSH, or lipoyl-DBT.

124. The method of any one of claims 119-123, further comprising measuring a level or activity of a mitochondrial protein and/or a nucleic acid encoding a mitochondrial protein in the cell sample and determining if the level or activity of the mitochondrial protein and/or the nucleic acid encoding the mitochondrial protein is decreased as compared to a level or activity of the mitochondrial protein and/or the nucleic acid encoding the mitochondrial protein of a cell sample not contacted with the test agent.

125. The method of claim 124, wherein the mitochondrial protein is FDX1, ALDHA1, ALDH2, LIAS, LIPT1, LIPT2, DLD, or Pyruvate dehydrogenase complex, 2-oxoglutarate dehydrogenase complex, Branched-Chain Alpha-Keto Acid Dehydrogenase Complex, and/or glycine cleavage.

126. The method of any one of claims 119-125, further comprising measuring a level of cell death in the cell sample and determining if the level of cell death is increased as compared to a level of cell death of a cell sample not contacted with the test agent.

127. A method of determining increased mitochondrial metabolism in a tumor and/or immune cell, comprising staining for lipoic acid in the tumor and/or immune cell.

128. A method of identifying a candidate anti-cancer agent, comprising the steps of:
(a) incubating a cell sample with copper-supplemented media;
(b) contacting a cell sample with a test agent;
(c) measuring cell viability of the cell sample; and
(d) identifying the test agent as a candidate anti-cancer agent if the level of cell viability is decreased as compared to a level of cell viability of a cell sample incubated with copper-supplemented media and not contacted with the test agent.

129. A method of identifying a candidate anti-cancer agent, comprising the steps of:
(a) incubating a cell sample with a copper chelator;
(b) contacting a cell sample with a test agent;
(c) measuring cell death of the cell sample; and
(d) identifying the test agent as a candidate anti-cancer agent if the level of cell death is decreased as compared to a level of cell death of a cell sample incubated a copper chelator and not contacted with the test agent.

130. A kit for identifying a candidate anti-cancer agent comprising a test agent and an assay for measuring cellular protein lipoylation.

131. A kit for identifying a candidate anti-cancer agent comprising copper-supplemented media, a test agent, and an assay for measuring cell viability.

132. A kit for identifying a candidate anti-cancer agent comprising a copper chelator, a test agent, and an assay for measuring cell death.
